(19) United States
(12) Patent Application Publication
(10) Pub. No.: US 2026/0256707 A1
(43) Pub. Date: Sep. 3, 2026

(54) LIPID FORMULATION USING MULTI-COMPONENT CONDENSATION REACTION AND LIPID NANOPARTICLES MADE USING THE SAME

(71) Applicant: Oregon State University, Corvallis, OR (US)

(72) Inventors: Gaurav Sahay, Portland, OR (US); Jonas Renner, Corvallis, OR (US); Milan Gautam, Corvallis, OR (US); Namratha Turuvekere Vittala Murthy, Portland, OR (US)

(73) Assignee: Oregon State University, Corvallis, OR (US)

(21) Appl. No.: 19/566,257

(22) Filed: Mar. 13, 2026

Related U.S. Application Data
(63) Continuation of application No. PCT/US2024/046430, filed on Sep. 12, 2024.
(60) Provisional application No. 63/582,670, filed on Sep. 14, 2023.
(60) Provisional application No. 63/595,209, filed on Nov. 1, 2023.

(51) Int. Cl. A61K 9/51; A61K 9/50; C07C 237/52; C07C 321/14; C07D 209/14; C07D 295/12

(52) U.S. Cl. CPC A61K 9/5123; A61K 9/5089; C07C 237/52; C07C 321/14; C07D 209/14; C07D 295/12

(57) ABSTRACT

Disclosed herein are aspects of a compound according to Formula I

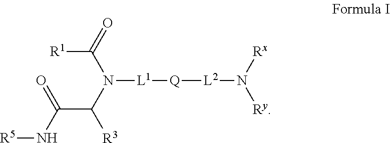

Also disclosed are compositions comprising the compounds that may be useful for delivering agents such as therapeutic and/or prophylactic agents, for example, nucleic acids such as, but not limited to, DNA or RNA, small molecules, proteins, polypeptides or peptides. In some aspects, the composition is a lipid nanoparticle. Also disclosed herein are lipid nanoparticles comprising the compounds and methods for making and using the nanoparticles.

### CROSS REFERENCE TO RELATED APPLICATION

This application is a continuation of International Application No. PCT/US2024/046430, filed Sep. 12, 2024, which in turn claims the benefit of the earlier filing date of U.S. Provisional Application No. 63/582,670, filed Sep. 14, 2023, and U.S. Provisional Application No. 63/595,209, filed Nov. 1, 2023, both of which are incorporated herein by reference in their entirety.

### ACKNOWLEDGMENT OF GOVERNMENT SUPPORT

This invention was made with government support under Grant Nos. R01 EY033423 and R01 HL146736 awarded by the National Institutes of Health. The government has certain rights in the invention.

### FIELD

Disclosed herein are novel lipid compounds, compositions comprising the compounds, and methods for making and using the compounds and compositions.

### BACKGROUND

Lipid-based transfection vehicles have played a significant role in facilitating the delivery of nucleic acids into cells, enabling numerous applications in research and clinical settings.

Traditional lipid-based transfection reagents, such as liposomes and lipidoids, have shown promising results; however, they often exhibit limitations in terms of transfection efficiency and target specificity.

### SUMMARY

Disclosed herein are aspects of a compound according to Formula I

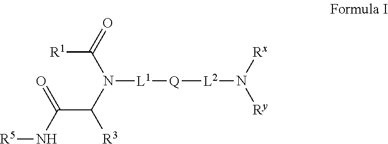

With respect to Formula I, L¹ is —(CH₂)ₙ₁—, L² is —(CH₂)ₙ₂—, and each n1 and n2 independently is from 1, 2, 3, 4, 5, or 6. Q is selected from —(CR^{a}₂)—, —NH—[(CH₂)ₐNH]_{b}—, —NR^{b}—, —(OCH₂CH₂)ₚO—, —P(═O)(CH₃)—, —S—S—, or

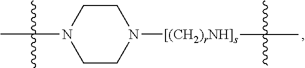

wherein each R^{a} independently is H or C₁₋₆alkyl, a is from 2 to 5, b is from 1 to 3, r is from 1 to 4, s is from 0 to 3, and p is from 1 to 5. Also with respect to Formula I, R^{x} is —C(═O)R² or —CH₂CH₂C(═O)OCH₂CH₂S—S—(CH₂)ₜCH₃, and R^{y} is

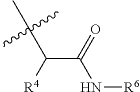

or —CH₂CH₂C(═O)OCH₂CH₂S—S—(CH₂)ₜCH₃, where each t independently is from 3 to 20. R^{b} is selected from H, C₁₋₆alkyl, or

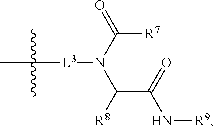

where L³ is —(CH₂)ₙ₃—, and n3 is from 1, 2, 3, 4, 5, or 6. In some aspects, each of n1 and n2, and n3 if present, is 2 or 3.

Additionally, if present, each of R¹, R² and R⁷ independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂. If present, each of R³, R⁴ and R⁸ independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂. And, if present, each of R⁵, R⁶, and R⁹ independently is selected from —C₄₋₂₂alkyl, —(CH₂)_{z+2}N(C₁₋₄alkyl)₂, —(CH₂)_{z+2}(5- or 6-membered heterocyclyl optionally substituted with C₁₋₄alkyl), —C₁₋₆OPG, —(CH₂)_{z+2}(5- to 9-membered heteroaryl), or —(CH₂)_{z+2}N((CH₂)_{z+1}OPG)₂, where PG is a silyl protecting group, such as TBS. And with respect to R¹-R⁹, x is from 0 to 10, y is from 1 to 5, and z is from 0 to 3.

In some aspects, the compound has a structure according to Formula II or Formula III.

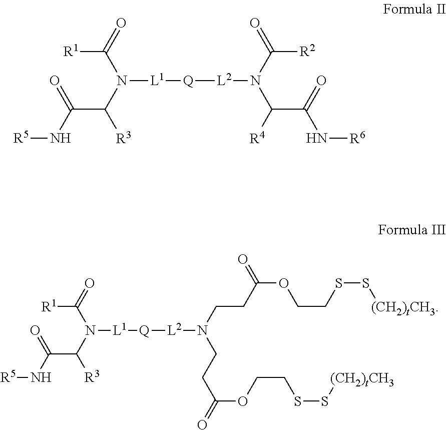

In some aspects, Q is —(CR^{a}₂)— and one R^{a} is H and the other R^{a} is C₁₋₆alkyl.

In some aspects, Q is —NH[(CH₂)ₐNH]_{b}—, a is 2, 3, or 4, and b is 1.

In some aspects, Q is —NR^{b}— and R^{b} is C₁₋₆alkyl, but in other aspects, R^{b} is

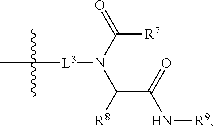

and n3 may be 2 or 3. In certain aspects, n1, n2 and n3 are the same as each other.

In some aspects, Q is

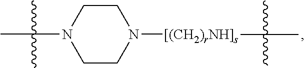

and in some aspects, s is 0, but in other aspects, s is 1 and r is 2.

In some aspects, Q is —(OCH₂CH₂)ₚO— and p is 1, 2, or 3.

In certain aspects, -L¹-Q-L²- is selected from: —(CH₂)₃NH(CH₂)₄NH(CH₂)₃—, —(CH₂)₂NH(CH₂)₄NH(CH₂)₂—, —(CH₂)₃NH(CH₂)₂NH(CH₂)₃—, —(CH₂)₂NH(CH₂)₂NH(CH₂)₂—, —(CH₂)₄N(CH₃)(CH₂)₄—, —(CH₂)₃N(CH₃)(CH₂)₃—, —CH₂C(CH₃)(CH₂)₃—,

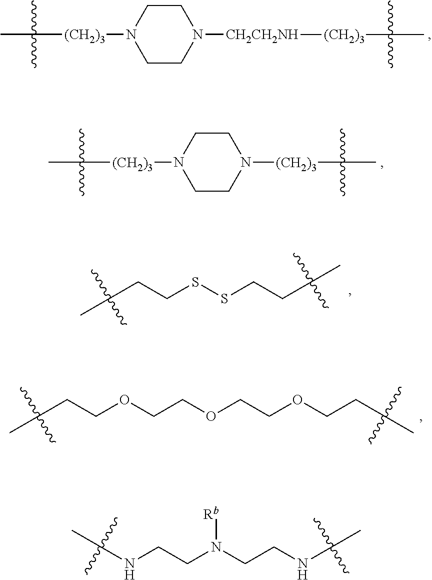

where R^{b} is

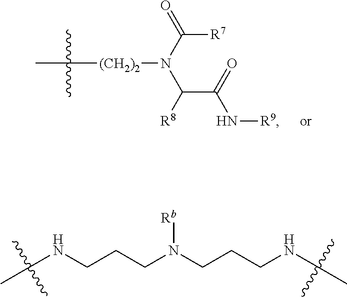

where R^{b} is

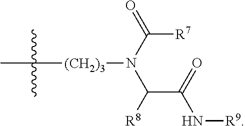

In any aspects, if present, each of R¹, R², and R⁷ independently may be selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, such as —C₁₀₋₂₀alkenyl, —(CH₂)ₓ—OC(═O)—(C₁₀₋₂₅alkyl), —(CH₂)ₓ—C(═O)O—(C₁₀₋₂₅alkyl), or —(CH₂)ₓ₊₂—S—S—(C₄₋₁₂alkyl), where x is from 0 to 6. In certain aspects, R¹, R², and R⁷, if present, are the same as each other.

In any aspects, if present, each of R³, R⁴, and R⁸, independently is selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, such as —C₁₀₋₂₀alkenyl, —(CH₂)ₓ—OC(═O)—(C₁₀₋₂₅alkyl), or —(CH₂)ₓ₊₂—S—S—(C₁₋₁₂alkyl), where x is from 0 to 6. In certain aspects, R³, R⁴, and R⁸, if present, are the same as each other.

In any aspects, if present, each of R⁵, R⁶, and R⁹, independently is selected from (CH₃)₂NCH₂CH₂CH₂—,

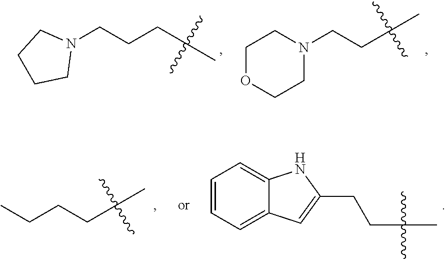

In certain aspects, R⁵, R⁶, and R⁹, if present, are the same as each other.

The disclosed compound may have a structure according to Formula IV, IV-A or IV-B as disclosed herein. With respect to Formulas IV, IV-A and IV-B, L¹, L² and L³ may be the same as each other, or at least one of L¹, L² and L³ is different from the other two. In some aspects, each of R¹, R² and R⁷, if present, are the same, each of R³, R⁴ and R⁸, if present, are the same, and/or if present, each of R⁵, R⁶ and R⁹ are the same.

Alternatively, the disclosed compound disclosed may comprise a Q moiety where Q is selected from —(CR^{a}₂)—, —NH—[(CH₂)ₐNH]_{b}—, —NR^{b}—, —(OCH₂CH₂)ₚO—, —P(═O)(CH₃)—, —S—S—, or

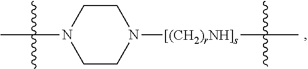

and R is selected from H, or C₁₋₆alkyl. In some aspects, the compound may have a structure according to any one of Formulas V-XX as disclosed herein. In some aspects of Formulas V-XX, R^{a} is C₁₋₃alkyl, R^{b} is C₁₋₃alkyl, a is 2, 3, or 4, b is 1, 2, or 3, r is 1, 2, or 3, s is 1, 2 or 3, and/or each t independently is from 3 to 15. In certain aspects of Formulas V-XX, R^{a} is methyl, R^{b} is methyl, a is 2, b is 1, r is 2, s is 1, and/or each t is 7.

Also disclosed herein are aspects of a composition comprising the compound. In some aspects, the compound is present in the composition in an amount of from 20 mol % to 100 mol %. The composition may be a nanoparticle and/or may further comprise an agent, such as a therapeutic or prophylactic agent. Typically, the agent is not covalently attached to the compound. The agent may be a nucleic acid, small molecule drug, protein, polypeptide, antibody, peptide, or a combination thereof. In some aspects, the agent is a nucleic acid and may be selected from a single stranded DNA, single stranded RNA, double-stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a combination thereof. The certain aspects, the agent is mRNA.

The composition further comprises a phospholipid, a structural lipid, a polymer-conjugated lipid, or a combination thereof. In some aspects, the composition comprises from greater than zero to 30 mol % of the phospholipid, from greater than zero to 70 mol % of the structural lipid, and/or from greater than zero to 10 mol % of the polymer-conjugated lipid.

Aspects of a pharmaceutical composition comprising the disclosed composition also are disclosed herein.

Also disclosed herein are aspects of a method for making the disclosed composition. In some aspects, the method comprises combining a first solution comprising a compound disclosed herein with a second solution comprising an agent, to form a mixture comprising a nanoparticle.

Aspects of a method for using the disclosed composition also are disclosed herein. In some aspects, the method comprises administering the composition to a subject, such as a human or animal subject. Administration may be by any suitable route, such as an intravenous, intramuscular, or intradermal route, and/or may comprise administering to lung and/or spleen tissue of the subject.

Also disclosed here are aspects of a use of the composition in the preparation of a medicament for administration to a subject.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a graph of size versus lipid nanoparticle (LNP) illustrating the average size of exemplary lipid nanoparticles containing the disclosed compounds.

FIG. 2 is a graph of polydispersity index (PDI) versus LNP illustrating the PDI of exemplary lipid nanoparticles containing the disclosed compounds.

FIG. 3 is a graph of encapsulation efficiency (EE) versus LNP illustrating the encapsulation efficiency of exemplary lipid nanoparticles containing the disclosed compounds.

FIG. 4 is a graph of zeta potential versus LNP illustrating the zeta potential of exemplary lipid nanoparticles containing the disclosed compounds.

FIGS. 5A-5C are cryo-electron microscopy images illustrating the shapes of lipid nanoparticles Thor-1 (T1, FIG. 5A), Thor-4 (T4, FIG. 5B) and Thor 11 (T11, FIG. 5C).

FIG. 6 provides in vivo bioluminescent images of BALB/c mice after treatment of 0.1 mg/kg fluc mRNA per mouse administration via intravenous injection 5-hour post-injection, and illustrating the in vivo mRNA transfection.

FIG. 7 is a graph of total flux versus LNP, illustrating the bioluminescent signals from the lung area using IVIS images.

FIGS. 8A-8F provide bioluminescent images of vital organs of BALB/c mice at 5 hours after mRNA treatment by tail vein injection (2 ug per mouse). FIGS. 8A-8E are ex vivo images of luciferase expression. FIG. 8F provides quantification of bioluminescent form ROI of lungs. All Data are presented as Mean±SD. An ordinary one-way ANOVA, with Tukey's correction was used for comparison with unmodified LNP. **p<0.01, ***p<0.001.

FIG. 9 is confocal microscopy images illustrating tdTomato expression in Ai9 mouse lungs cross-sections after cre mRNA encapsulated T5 LNPs delivery by tail vein injection (2 ug cre mRNA per mouse). Lungs from the PBS and Cre-T5 LNPs treated mice were harvested after 10 days of injection, DAPI stained and observed by confocal microscopy.

FIG. 10 is confocal microscopy images illustrating tdTomato expression in Ai9 mouse spleen cross-sections after cre mRNA encapsulated T5 LNPs delivery by tail vein injection (2 ug cre mRNA per mouse). Spleen from the PBS and Cre-T5 LNPs treated mice were harvested after 10 days of injection, DAPI stained and observed by confocal microscopy.

### DETAILED DESCRIPTION

### I. Terms and Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing aspects from discussed prior art, the aspect numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

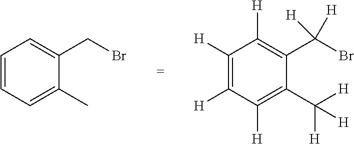

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

The compounds according to the present disclosure may be in a free base form, i.e., not in a salt form, or the compounds may be in a salt form, such as a pharmaceutically acceptable salt as defined herein. A person of ordinary skill in the art will understand that in such a salt form the compound may have either a negative or a positive charge, and/or may include a counter ion, such as an organic and/or inorganic counter ion as known to a person of ordinary skill in the art, and/or as described herein. In some aspects, the compound may be in a zwitterion form having both a positive and negative charge. The overall change of such a compound may be zero and/or it may not have a separate counter ion.

Additionally, or alternatively, the disclosed compound may be in a non-solvated form or it may be solvated, as defined herein.

The compounds of the present disclosure may also contain unnatural proportions of atomic isotopes at one or more of atoms that constitute such compounds. For example, the compounds may be radiolabeled with radioactive isotopes, such as, for example, tritium (3H), iodine-125 (125I) or carbon-14 (14C), and/or contain an unnatural proportion of non-radioactive isotopes, such as deuterium, or carbon-13 (C¹³). All isotopic variations of the compounds of the present disclosure, whether radioactive or not, are encompassed within the scope of the present disclosure.

In some aspects, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to 5 hydrogens are replaced by deuterium, such as in C₂DₓH₅₋ₓ.

In some aspects, one or more carbon atoms present in a compound may be replaced with a carbon-13 or carbon-14. Thus, a recitation of alkyl includes carbon-13 and/or carbon-14 alkyl, where from one to the maximum number of carbon atoms present may be replaced by either carbon-13 or carbon-14. For example, ethyl may be C₂H₅ or C₂H₅ where one or both carbons are replaced by carbon-13, carbon-14, or a mixture thereof.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds may exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms.

Any group or moiety may be optionally substitute, i.e., may be substituted or unsubstituted, unless otherwise specified, for example, as “unsubstituted” or “substituted.” In particular aspects, the group or moiety may or may not be expressly defined as substituted, but is still contemplated to be optionally substituted. A substituted group or moiety has at least one, and may be two or more, hydrogen atoms of the specified group or moiety independently replaced with the same or different substituents groups. Unless otherwise specified, suitable substituents include halogen, ═O, —C(O)OR, —SH, —SSR, —NHC(O)NR₂, P(═O)(OR)₂, —OR, —OC(O)NR₂, —NHC(O)OR, ═NNHC(O)R, —SO₂NR₂, ketal, thioketal, —OC(O)OR, —NHCSNR₂, ═NOR, —C(OR)₃, OH, NR₂, carbonyl (C═O), aldehyde, alkyl, alkenyl, alkynyl, and straight chain, cyclic and branched versions thereof, where each R independently is H, alkyl, alkenyl, alkynyl, phenyl, or heteroaryl, or 2 R's together with the atoms to which they are attached, form a 3- to 8-membered heterocyclyl or heteroaryl comprising from 1, 2, or 3 heteroatoms selected from N, O and S. Typically, each R independently is H, C₁₋₈alkyl, C₆aryl, 3- to 8-membered heteroaryl comprising from 1, 2, or 3 heteroatoms selected from N, O and S, or 2 R's together with the atoms to which they are attached, form a 3- to 8-membered heterocyclyl or heteroaryl. In some aspects, each R independently is H or C₁₋₆alkyl.

In some aspects, a group that is substituted has at least one substituent up to the number of substituents possible for a particular moiety, such as 1 substituent, 2 substituents, 3 substituents, or 4 substituents.

Additionally, in aspects where a group or moiety is substituted with a substituted substituent, the nesting of such substituted substituents is limited to three, thereby preventing the formation of polymers. Thus, in a group or moiety comprising a first group that is a substituent on a second group that is itself a substituent on a third group, which is attached to the parent structure, the first (outermost) group can only be substituted with unsubstituted substituents. For example, in a group comprising -(heteroaryl-1)-(heteroaryl-2)-(heteroaryl-3), heteroaryl-3 can only be substituted with substituents that are not themselves substituted.

And the term “substituted” refers to all subsequent modifiers in a term, for example in the term “substituted “-alkylheteroaryl,” substitution may occur on the “alkyl” portion, the “heteroaryl” portion or both portions of the alkylheteroaryl group.

“Alkyl” refers to a saturated aliphatic hydrocarbyl group having, unless otherwise specified, from 1 to 30 (C₁₋₃₀) or more carbon atoms, such as from 1 to 10 (C₁₋₁₀) carbon atoms, from 1 to 6 (C₁₋₆) carbon atoms, or from 6 to 30 carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), hexyl (C₆H₁₃), heptyl (C₇H₁₅), octyl (C₈H₁₇), decyl (C₁₀H₂₁), dodecyl (C₁₂H₂₅), tetradecyl (C₁₄H₂₉), hexadecyl (C₁₆H₃₃), heptadecyl (C₁₇H₃₅), octadecyl (C₁₈H₃₇), or eicosanyl (C₂₀H₄₁).

“Alkenyl” refers to an unsaturated aliphatic hydrocarbyl group having, unless otherwise specified, at least 1 double bond and from 2 to 30 (C₂₋₃₀) or more carbon atoms, such as from 2 to 10 (C₂₋₁₀) carbon atoms, from 2 to 6 (C₂₋₆) carbon atoms, or from 6 to 30 carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkenyl moiety may be substituted or unsubstituted. This term includes, by way of example, linear and branched hydrocarbyl groups, such as vinyl, allyl, or but-3-en-1-yl. Also included within this term are the cis and trans isomers or mixtures of these isomers, unless otherwise specified.

“Alkynyl” refers to an unsaturated aliphatic hydrocarbyl group having, unless otherwise specified, at least 1 triple bond and from 2 to 30 (C₂₋₃₀) or more carbon atoms, such as from 2 to 10 (C₂₋₁₀) carbon atoms, from 2 to 6 (C₂₋₆) carbon atoms, or from 6 to 30 carbon atoms such as from 6-25 carbon atoms, from 6-22 carbon atoms, from 6-20 carbon atoms, from 6-18 carbon atoms, from 8-20 carbon atoms, from 8-18 carbon atoms, from 10-20 carbon atoms, from 12-20 carbon atoms, from 14-20 carbon atoms, from 14-18 carbon atoms, or from 16-18 carbon atoms. An alkynyl moiety may be substituted or unsubstituted. This term includes, by way of example, linear and branched hydrocarbyl groups, such as ethynyl, 1-propynyl and 2-propynyl.

“Heteroaryl” refers to an aromatic group or moiety of, unless specified otherwise, from 5 to 15 ring atoms comprising at least one carbon atom and at least one heteroatom, such as N, S, or O. A heteroaryl group or moiety may comprise a single ring (e.g., pyridinyl, pyrimidinyl or pyrazolyl) or multiple condensed rings (e.g., indolyl, benzopyrazolyl, or pyrazolopyridinyl). Heteroaryl groups or moiety may be, for example, monocyclic, bicyclic, tricyclic or tetracyclic. Unless otherwise stated, a heteroaryl group or moiety may be substituted or unsubstituted.

As used herein, “heterocyclyl” refers to non-aromatic ring systems, and more specifically refers to a stable three- to fifteen-membered ring moiety comprising carbon atoms and at least one, such as from one to five heteroatoms selected from O, N, and S. The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and the nitrogen, phosphorus, carbon, silicon or sulfur atoms in the heterocyclyl moiety can be optionally oxidized to various oxidation states. For convenience, nitrogens, particularly but not exclusively, those defined as annular aromatic nitrogens, are meant to include their corresponding N-oxide form, although not explicitly defined as such in a particular example. Exemplary heterocyclyl groups include, but are not limited to, 4-piperidonyl, pyrrolidinyl, pyrazolidinyl, imidazolinyl, piperazinyl, piperidinyl, morpholinyl, homopiperazinyl, homopiperidinyl, aziridine, azetidine, oxirane, thiirane, lactam, lactone, thietane, tetrahydopyran, azocane, oxepane, quinuclidine, azaadmantane, indoline, dihydroquinoline, thiomorpholine, thiane, tetrahydrofuran, tetrahydropyran, or 1,3-dioxalane.

“Lipid” refers to an organic compound that is readily soluble in nonpolar solvents such as hydrocarbons, but typically is sparingly or non-soluble in water, and may be poorly soluble in other polar solvents. Ionizable lipids are lipids that can be ionized, for example, with pH-dependent ionization. The lipid may be anionic and/or cationic, for example, it may form an anion and/or a cation depending on pH. In some aspects, an ionizable lipid may be positive at low pH, and may be substantially neutral at physiological or neutral pH.

“Nanoparticle” as used herein refers to a composition, such as a pharmaceutical formulation, having a particle size (for example, a diameter) of from 1 to 1000 nanometers, such as from 1 to 500 nanometers or from 1 to 100 nanometers, and incorporating one or more lipid compounds disclosed herein. Nanoparticle compositions include, but are not limited to, lipid nanoparticles (LNPs), liposomes (e.g., lipid vesicles), and lipoplexes.

“Lipid nanoparticle” (LNP) refers to a nanoparticle comprising one or more lipid compounds. Typically, the lipid compound(s) will be a major component of the nanoparticle. LNPs may be substantially spherical in shape. Disclosed LNPs may be positively charged in low pH and substantially neutral at physiological pH. Alternatively, the LNP may be uncharged, even if the lipids themselves are charged. In some aspects, the ionizable lipid is contained in the core and its charge may be shielded by other lipid components.

“Nucleic acid” refers to a polynucleotide molecule. The polynucleotide may be a naturally occurring polynucleotide or a synthetic polynucleotide. A nucleic acid may be a DNA, RNA or mixture of DNA and RNA nucleotides. Typically, the nucleic acid contains from 20 to 10,000 nucleotides or more, such as from 100, 200, 300, 400, 500, 600, 700, 800, 900, 1000, 2000, 3000, 4000, or 5000 nucleotides to 10,000 nucleotides.

Exemplary nucleic acids include, but are not limited to, single stranded DNA, single stranded RNA, double stranded DNA, RNA-RNA hybrid, DNA-RNA hybrid, shortmer, antagomir, antisense, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), or a combination thereof.

“Peptide” refers to a compound comprising amino acid residues connected by peptide bonds. Typically, a peptide compound has from 2 to about 50 amino acid residues.

“Polypeptide” refers to a compound comprising amino acid residues connected by peptide bonds. When the amino acids are alpha-amino acids, either the L-optical isomer or the D-optical isomer can be used. In some aspects, a polypeptide has from about 50 amino acid residues to 2000 or more amino acid residues.

“Protein” refers to a molecule or complex comprising one or more polypeptides having secondary, tertiary and/or quaternary structure. The secondary, tertiary and/or quaternary structure of a protein typically is stabilized using non-covalent bonds, such as ionic bonds, hydrogen bonds, hydrophobic interactions, and/or van der Walls interactions. Additionally, or alternatively, a protein may include disulfide bonds, such as between the thiol groups of cysteine residues.

“Small Molecule” refers to a organic molecule having a molecular weight of about 2000 Daltons or less. In some aspects, the term “small molecule” refers to a compound that is not a polypeptide, protein, or nucleic acid molecule. A small molecule may be a small molecule therapeutic and/or prophylactic, such as an antibiotic, anti-inflammatory, anticancer, antiviral, immunosuppressant, analgesic, antifungal, antiparasitic, anticonvulsants, antidepressant, anti-anxiety, anti-psychotic, and the like.

“Pharmaceutically acceptable excipient” refers to a substantially physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, as a carrier, flavoring, thickener, diluent, buffer, preservative, or surface active agent and/or to modify properties of a pharmaceutical composition. Examples of excipients include, but are not limited, to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

“Pharmaceutically acceptable salt” refers to pharmaceutically acceptable salts of a compound that are derived from a variety of organic and inorganic counter ions as will be known to a person of ordinary skill in the art and typically include, by way of example only, sodium, potassium, calcium, magnesium, ammonium, tetraalkylammonium, hydrochloride, hydrobromide, tartrate, mesylate, acetate, maleate, oxalate, and the like. In particular, the disclosed compounds may form salts with a variety of pharmaceutically acceptable acids, including, without limitation, inorganic acids such as hydrochloric acid, hydrobromic acid, sulfuric acid, nitric acid, phosphoric acid, and the like, as well as organic acids such as formic acid, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, benzene sulfonic acid, isethionic acid, methanesulfonic acid, ethanesulfonic acid, p-toluenesulfonic acid, salicylic acid, and the like. (See, for example, S. M. Berge, et al., “Pharmaceutical Salts,” J. Pharm. Sci., 1977; 66:1-19 which is incorporated herein by reference.)

“Silyl protecting group” refers to a protecting group containing a silyl moiety, typically suitable to protect a hydroxyl (OH) moiety. Exemplary silyl protecting groups include, but are not limited to, trimethylsilyl (TMS), triethylsilyl (TES), isopropyldimethylsilyl (IPDMS), diethylisopropylsilyl (DEIPS), t-butyldimethylsilyl (TBS), t-Butyldiphenylsilyl (TBDPS), triisopropylsilyl (TIPS), tetraisopropyldisiloxanylidene (TIPDS), or di-t-butylsilylene (DTBS).

“Solvate” refers to a complex formed by combination of solvent molecules with molecules or ions of the solute. The solvate may be a hydrate.

“Subject” refers to mammals and other animals, particularly humans. Thus, disclosed methods are applicable to both human therapy and veterinary applications.

### II. Compounds

Disclosed herein are aspects of a compound that may be suitable for use in delivery applications for one or more therapeutic agents, such as nucleic acid agents such as mRNA, small molecule drug, protein, polypeptide, antibody, peptide and the like. In some aspects, the compound is a lipid, and may be an ionizable lipid. And/or in some aspects, the therapeutic agent is mRNA.

In some aspects, delivery applications comprising the disclosed compounds demonstrate improved in vitro transfection, compared to delivery applications comprising existing lipids. And the disclosed compounds exhibit low toxicity in a cytotoxicity assay. And in some aspects, compositions comprising the disclosed compounds may selectively target organs, such as the lungs and/or spleen, but in other aspects, the compositions may be systemic.

In some aspects, the compound has a structure according to formula I.

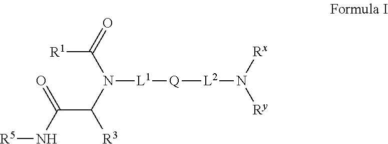

With respect to Formula I, L¹ is —(CH₂)ₙ₁—, and L² is —(CH₂)ₙ₂—;
each n1 and n2 independently is from 1, 2, 3, 4, 5, or 6;
Q is selected from —(CR^{a}₂)—, —NH—[(CH₂)ₐNH]_{b}—, —NR^{b}—, —(OCH₂CH₂)ₚO—, —P(═O)(CH₃)—, —S—S—, or

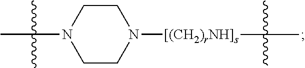

each R^{a} independently is H or C₁₋₆alkyl;
R^{x} is —C(═O)R² or —CH₂CH₂C(═O)OCH₂CH₂S—S—(CH₂)ₜCH₃;
R^{y} is

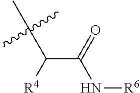

or —CH₂CH₂C(═O)OCH₂CH₂S—S—(CH₂)ₜCH₃;
a is from 2 to 5;
b is from 1 to 3;
r is from 1 to 4;
s is from 0 to 3;
p is from 1 to 5;
t is from 3 to 20;
R^{b} is selected from H, C₁₋₆alkyl, or

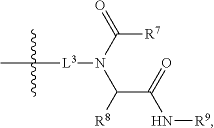

where L³ is —(CH₂)ₙ₃— and n3 is from 1, 2, 3, 4, 5, or 6;
each of R¹, R² and R⁷, if present, independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂; each of R³, R⁴ and R⁸, if present, independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂; each of R⁵, R⁶, and R⁹, if present, independently is selected from —C₄₋₂₂alkyl, —(CH₂)_{z+2}N(C₁₋₄alkyl)₂, —(CH₂)_{z+2}(5- or 6-membered heterocyclyl optionally substituted with C₁₋₄alkyl), —C₁₋₆OPG (such as —C₁₋₆OTBS), —(CH₂)_{z+2}(5- to 9-membered heteroaryl), or —(CH₂)_{z+2}N((CH₂)_{z+1}OPG)₂ (such as —(CH₂)_{z+2}N((CH₂)_{z+1}OTBS)₂), where PG is a silyl protecting group, such as TBS.
x is from 0 to 10;
y is from 1 to 5; and
z is from 0 to 3.

In some aspects, each t independently is from 3 to 15, such as from 3 to 12. In certain aspects, each t independently is 4, 5, 6, 7, 8, 9, or 10, such as 5, 6, 7, 8, or 9 and may be 6, 7 or 8.

In particular aspects, each t is 7.

In any aspects of R¹, R², R³, R⁴, R⁷, and R⁸ that comprise an alkenyl moiety, the alkenyl moiety may comprise one double bond, or more than one double bond, such as 1, 2, 3, 4, or more double bonds, or 1, 2, 3, or 4, or 1, 2, or 3, or 1 or 2 double bonds. In some aspects, an alkenyl moiety contains 1 double bond. In other aspects, an alkenyl moiety contains 2 double bonds. In other aspects, an alkenyl moiety contains 3 double bonds. In any aspects, an alkenyl moiety may have a cis configuration, a trans configuration, or a mixture thereof. In some aspects of an alkenyl moiety than comprises more than one double bond, all double bonds have the same configuration, such as all cis or all trans.

In some aspects of Formula I, the compound has a structure according to Formula II:

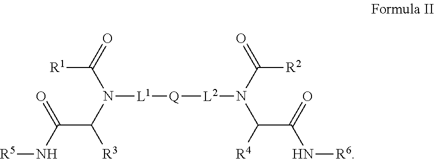

In some aspects of Formula I, the compound has a structure according to Formula III:

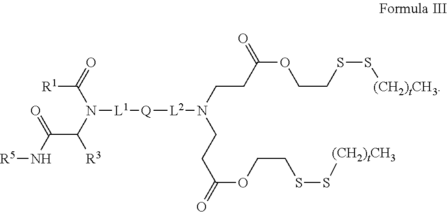

In some aspects of Formulas I, II, or III, Q is —(CR^{a}₂)— and in certain aspects, one R^{a} is H and the other R^{a} is C₁₋₆alkyl, such as C₁₋₃alkyl, and may be methyl. In some such aspects, each of n1 and n2 independently is 1, 2, or 3. In certain aspects, one of n1 and n2 is 1 and the other of n1 and n2 is 3. In particular aspects, n1 is 1 and n2 is 3.

In some aspects of Formulas I, II, or III, Q is —NH—[(CH₂)ₐNH]_{b}—. In certain aspects, a is 2, 3, or 4, and may be 2 or 4. In some aspects, b is 1 or 2, and in certain aspects, b is 1. In particular aspects, b is 1 and a is 2, 3, or 4, such as 2 or 4. And in some aspects when Q is —NH—[(CH₂)ₐNH]_{b}— each of n1 and n2 independently is 2, 3 or 4, and may be 3.

In some aspects of Formulas I, II, or III, Q is —NR^{b}—. In some such aspects, R^{b} is C₁₋₆alkyl, such as C₁₋₃alkyl and may be methyl. In other aspects, R^{b} is

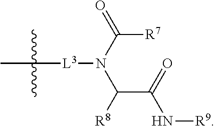

In some such aspects, n3 is 2 or 3 and may be 2. In some aspects when Q is —NR^{b}—, each of n1 and n2 independently is 2, 3 or 4, such as 2 or 3, and may be the same. And in some aspects where R^{b} is

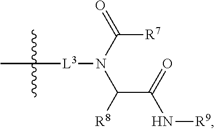

each of n1, n2 and n3 are 2, 3 or 4, such as 2 or 3, and may be the same as each other.

In some aspects of Formulas I, II, or III, Q is

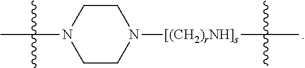

In some aspects, s is 0. In other aspects, s is 1 and r is 2.

In some aspects of Formulas I, II, or III, Q is —S—S—.

In some aspects of Formulas I, II, or III, Q is —(OCH₂CH₂)ₚO— where p is 1, 2, 3, 4, or 5, such as 1, 2, or 3.

Exemplary -L¹-Q-L²- moieties include, but are not limited to: —(CH₂)₃NH(CH₂)₄NH(CH₂)₃—, —(CH₂)₂NH(CH₂)₄NH(CH₂)₂—, —(CH₂)₃NH(CH₂)₂NH(CH₂)₃—, —(CH₂)₂NH(CH₂)₂NH(CH₂)₂—, —(CH₂)₄N(CH₃)(CH₂)₄—, —(CH₂)₃N(CH₃)(CH₂)₃—, —CH₂C(CH₃)(CH₂)₃—,

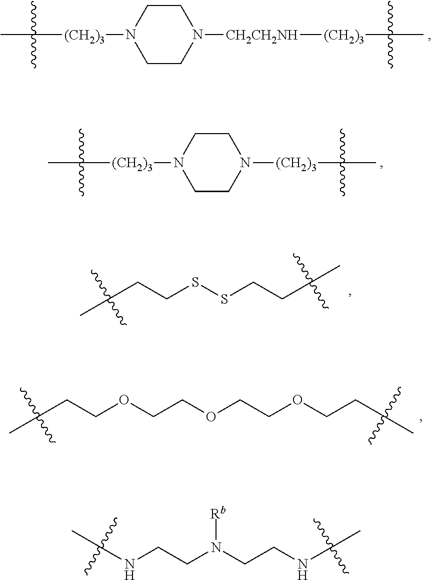

where R^{b} is

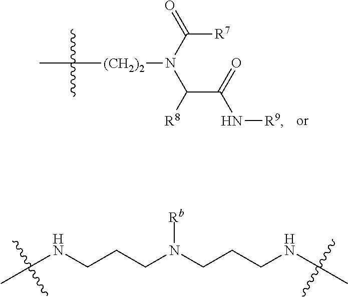

where R^{b} is

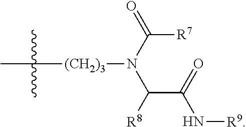

In some aspects, each of R¹, R², and R⁷, if present, independently is selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, where x is from 0 to 6. In certain aspects, each of R¹ and R², and R⁷, if present, independently is selected from —C₁₀₋₂₀alkenyl, —(CH₂)ₓ—OC(═O)—(C₁₀₋₂₅alkyl), —(CH₂)ₓ—C(═O)O—(C₁₀₋₂₅alkyl), or —(CH₂)ₓ₊₂—S—S—(C₄₋₁₂alkyl), where x is from 0 to 6.

Exemplary R¹, R², and/or R⁷ moieties include, but are not limited to:

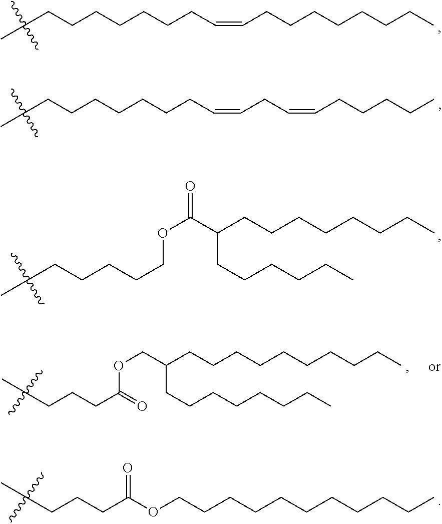 [SMILES: *CCCCCCC/C=C\CCCCCCCC ; *CCCCCCC/C=C\C/C=C\CCCCC ; *CCCCCOC(=O)C(CCCCCC)CCCCCCCC ; *CCCC(=O)OCC(CCCCCCCC)CCCCCCCCCC ; *CCCC(=O)OCCCCCCCCCCC]

In some aspects, R¹, R², and R⁷, if present are the same. But in other moieties, one or more of R¹, R², and R⁷, if present, are different.

In some aspects, each of R³, R⁴, and R⁸, if present, independently is selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, where x is from 0 to 6. In some aspects, each of R³ and R⁴, and R⁸, if present, independently is selected from —C₁₀₋₂₀alkenyl, —(CH₂)ₓ—OC(═O)—(C₁₀₋₂₅alkyl), or —(CH₂)ₓ₊₂—S—S—(C₁₋₁₂alkyl), where x is from 0 to 6.

Exemplary R³, R⁴, and/or R⁸ moieties include, but are not limited to:

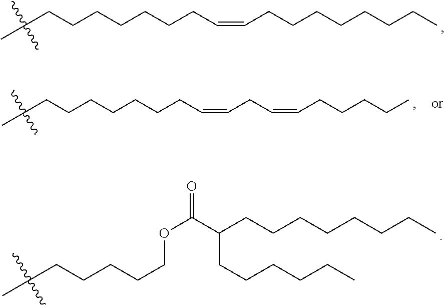 [SMILES: *CCCCCCC/C=C\CCCCCCCC ; *CCCCCCC/C=C\C/C=C\CCCCC ; *CCCCCOC(=O)C(CCCCCC)CCCCCCCC]

In some aspects, R³, R⁴, and R⁸, if present, are the same. But in other moieties, one or more of R³, R⁴, and R⁸, if present, are different.

In some aspects, each of R⁵, R⁶, and R⁹, if present, independently is selected from (CH₃)₂NCH₂CH₂CH₂—,

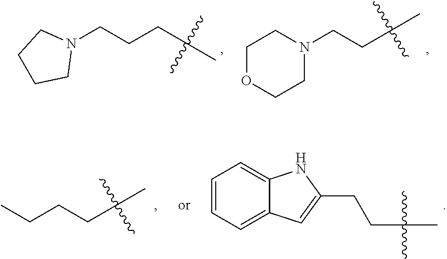

In some aspects, the compound has a structure according to Formula IV, IV-A or IV-B:

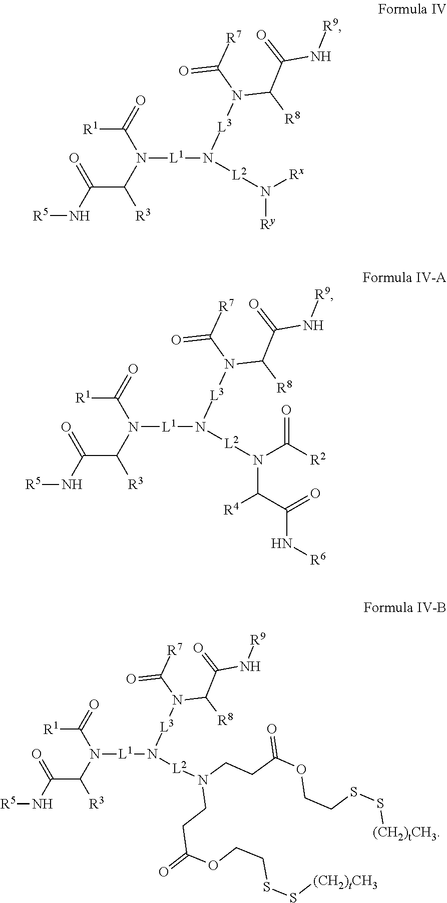

With respect to Formulas IV, IV-A, and IV-B, R¹, R², R³, R⁴, R⁵, R⁶, R⁷, R⁸, R⁹, R^{x}, R^{y}, L¹, L², L³ and t are as previously defined for Formula I.

In some aspects of Formulas IV, IV-A, and IV-B, at least one of L¹, L² and L³ is different from the other two.

In some aspects of Formulas IV, IV-A, and IV-B, all of L¹, L² and L³ are the same. That is, n1, n2 and n3 are the same. In some aspects, n1, n2, and n3 are 2, 3, or 4, such as 2, or 3.

In some aspects of Formulas IV, IV-A, and IV-B, each of R¹, R² and R⁷, if present, are the same.

In some aspects of Formulas IV, IV-A, and IV-B, each of R³, R⁴ and R⁸, if present, are the same.

In some aspects of Formulas IV, IV-A, and IV-B, each of R⁵, R⁶ and R⁹, if present, are the same.

In certain aspects of Formulas IV, IV-A, and IV-B, each of R¹, R² and R⁷, if present, are the same as each other, each of R³, R⁴ and R⁸, if present, are the same as each other, and each of R⁵, R⁶ and R⁹, if present, are the same as each other.

In some aspects of Formulas I-III, Q is selected from —(CR^{a}₂)—, —NH[(CH₂)ₐNH]_{b}—, —NR^{b}—, —(OCH₂CH₂)ₚO—, —P(═O)(CH₃)—, —S—S—, or

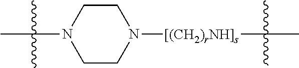

where R^{b} is selected from H, or C₁₋₆alkyl. In some such aspects, the compound has a structure according to a formula selected from:

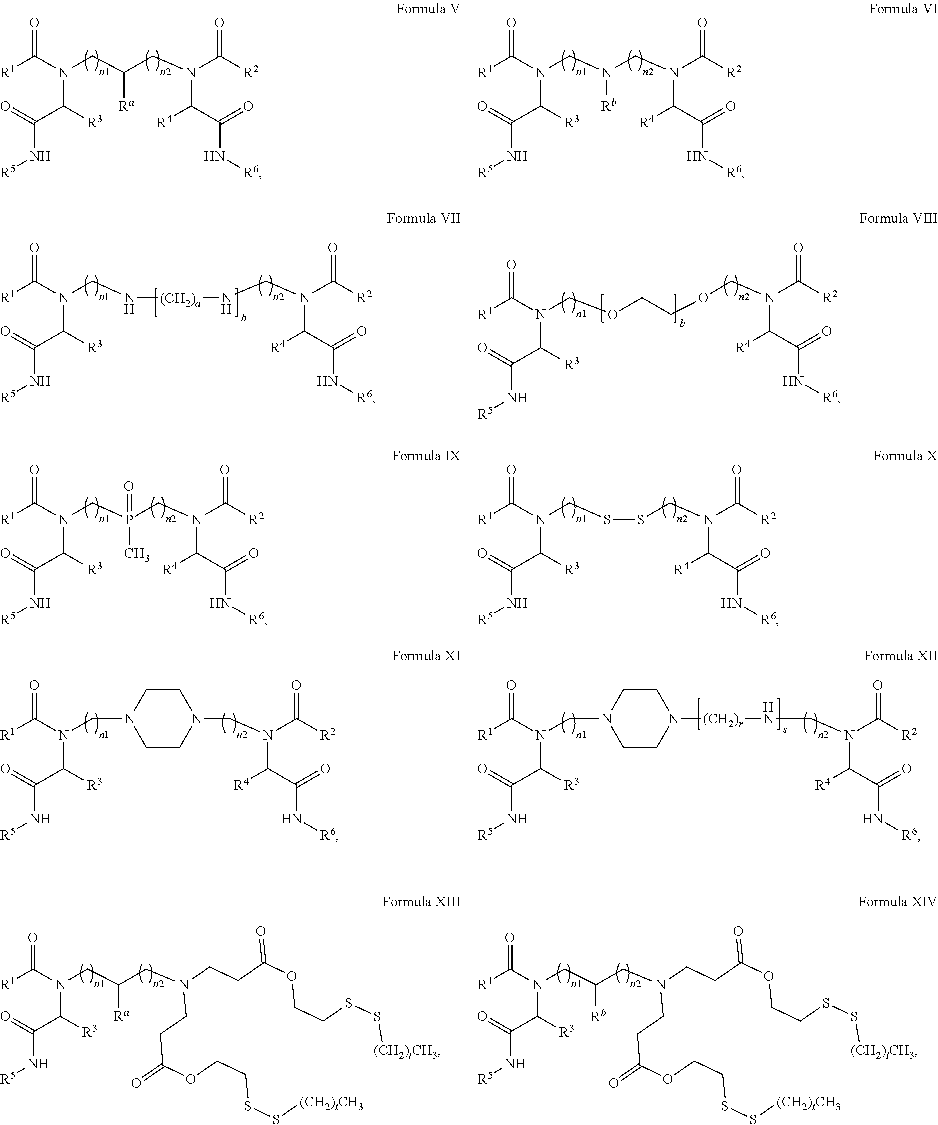

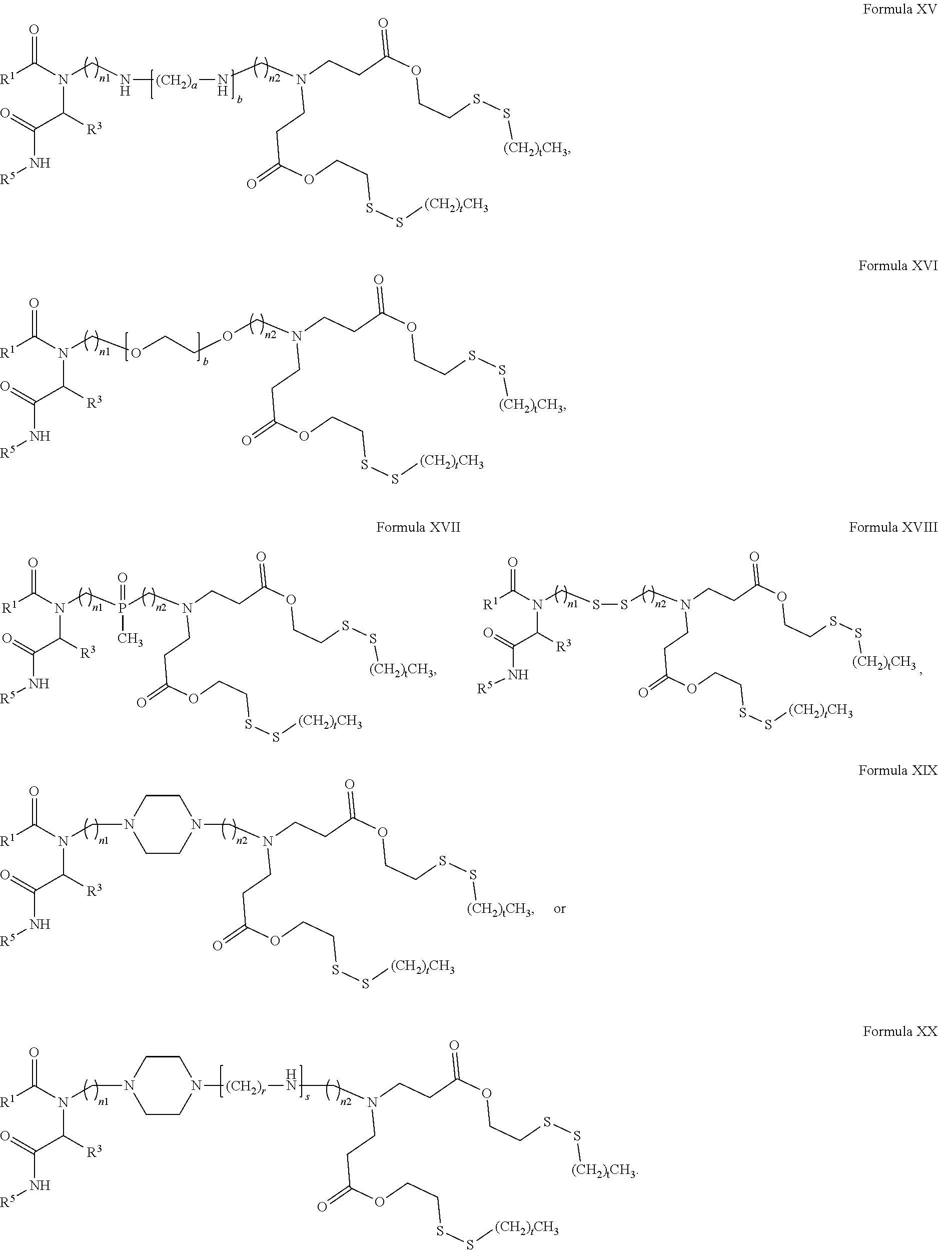

With respect to Formulas V-XX, n1, n2, R^{a}, R¹, R², R³, R⁴, R⁵, R⁶, a, b, r, s and t, if present, are as previously defined for Formula I, and R^{b} is C₁₋₆alkyl.

In some aspects of Formulas V and XIII, R^{a} is C₁₋₃alkyl, and may be methyl. In some aspects, each of n1 and n2 independently is 1, 2, or 3. In certain aspects, n1 is 1 and n2 is 3.

In some aspects of Formulas VI and XIV, R^{b} is C₁₋₃alkyl and may be methyl. In some aspects, each of n1 and n2 independently is 2, 3, or 4, such as 2 or 3. In some aspects, n1 and n2 are the same, but in other aspects, n1 and n2 are different. In a particular aspect, n1 and n2 are both 2. In another particular aspect, n1 and n2 are both 3.

In some aspects of Formulas VII and XV, a is 2, 3, or 4. In one aspect, a is 2. In another aspect, a is 3 and in further aspect, a is 4. In some aspects, b is 1 or 2, and in particular aspects, b is 1. In some aspects, each of n1 and n2 independently is 2, 3, or 4, such as 2 or 3. In some aspects, n1 and n2 are the same, but in other aspects, n1 and n2 are different. In particular aspects, both n1 and n2 are both 3.

In some aspects of Formulas VIII and XVI, b is 1, 2, or 3, such as 2 or 3, and may be 2. In some aspects, each of n1 and n2 independently is 2, 3, or 4, such as 2 or 3. In some aspects, n1 and n2 are the same, but in other aspects, n1 and n2 are different. In particular aspects, both n1 and n2 are both 2.

In some aspects of Formulas IX and XVII, each of n1 and n2 independently is 2, 3, or 4, such as 2 or 3. In some aspects, n1 and n2 are the same, but in other aspects, n1 and n2 are different.

In some aspects of Formulas X and XVIII, each of n1 and n2 independently is 2, 3, or 4, such as 2 or 3. In some aspects, n1 and n2 are the same, but in other aspects, n1 and n2 are different. In particular aspects, both n1 and n2 are both 2.

In some aspects of Formulas XI and XIX, each of n1 and n2 independently is 2, 3, or 4, such as 2 or 3. In some aspects, n1 and n2 are the same, but in other aspects, n1 and n2 are different. In particular aspects, both n1 and n2 are both 3.

In some aspects of Formulas XII and XX, r is 1, 2, or 3, such as 2 or 3 and in some aspects, r is 2. In some aspects, s is 1, 2 or 3, such as 1 or 2, and in certain aspects, s is 1. In some aspects, each of n1 and n2 independently is 2, 3, or 4, such as 2 or 3. In some aspects, n1 and n2 are the same, but in other aspects, n1 and n2 are different. In particular aspects, both n1 and n2 are both 2.

In some aspects of Formulas XIII to XX, t is from 3 to 15, such as from 3 to 12. In certain aspects, t is 4, 5, 6, 7, 8, 9, or 10, such as 5, 6, 7, 8, or 9 and may be 6, 7 or 8. In particular aspects, t is 7.

Exemplary compounds according to Formula I include but are not limited to:

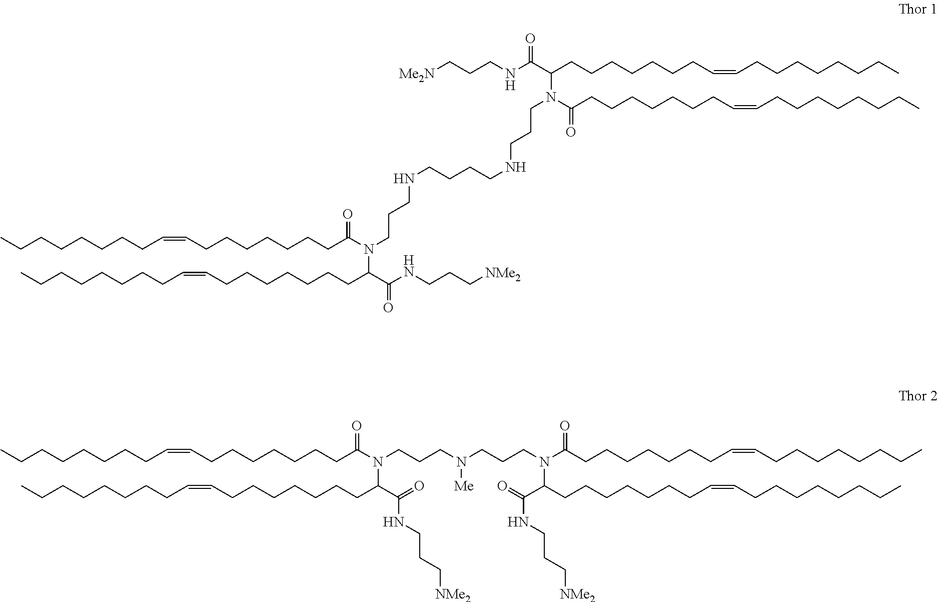

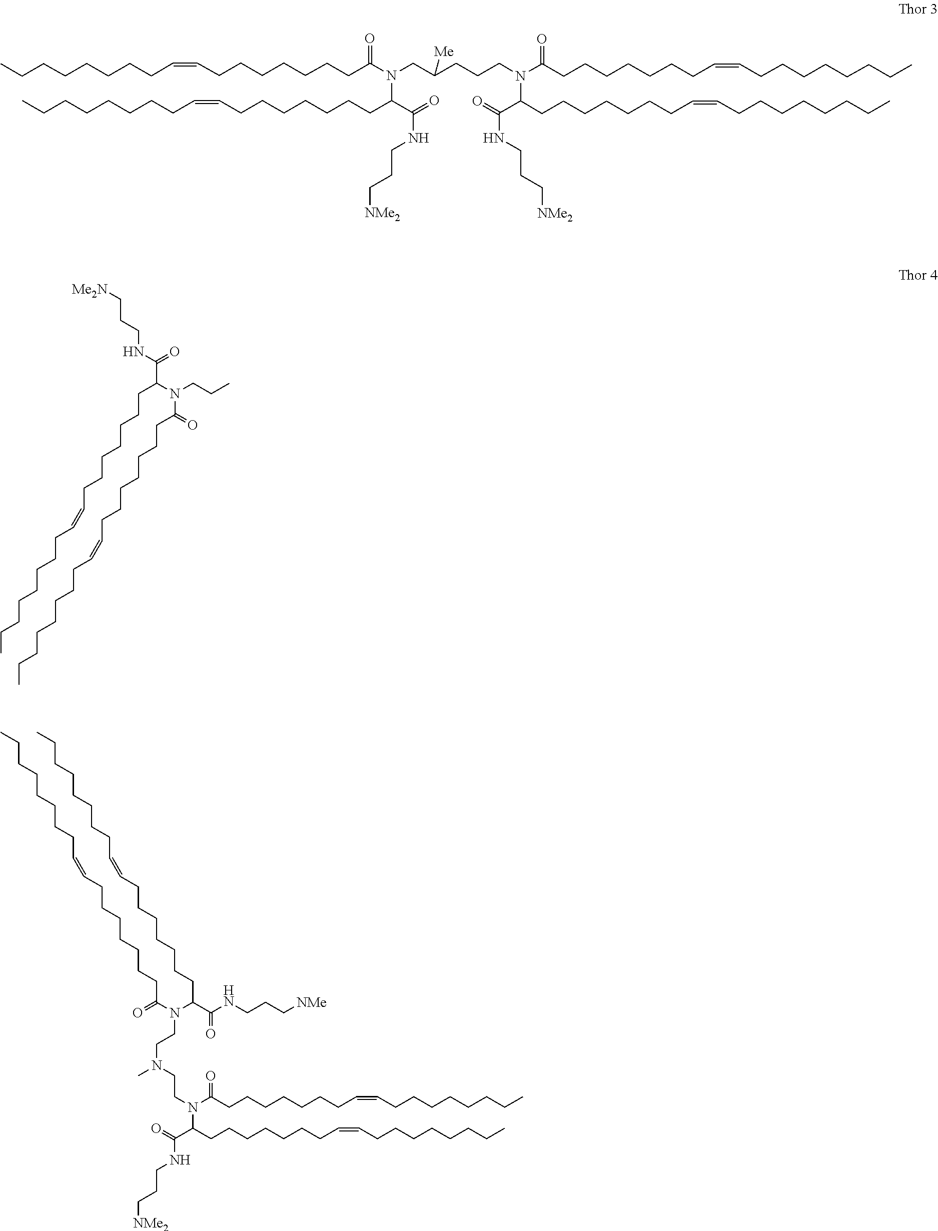

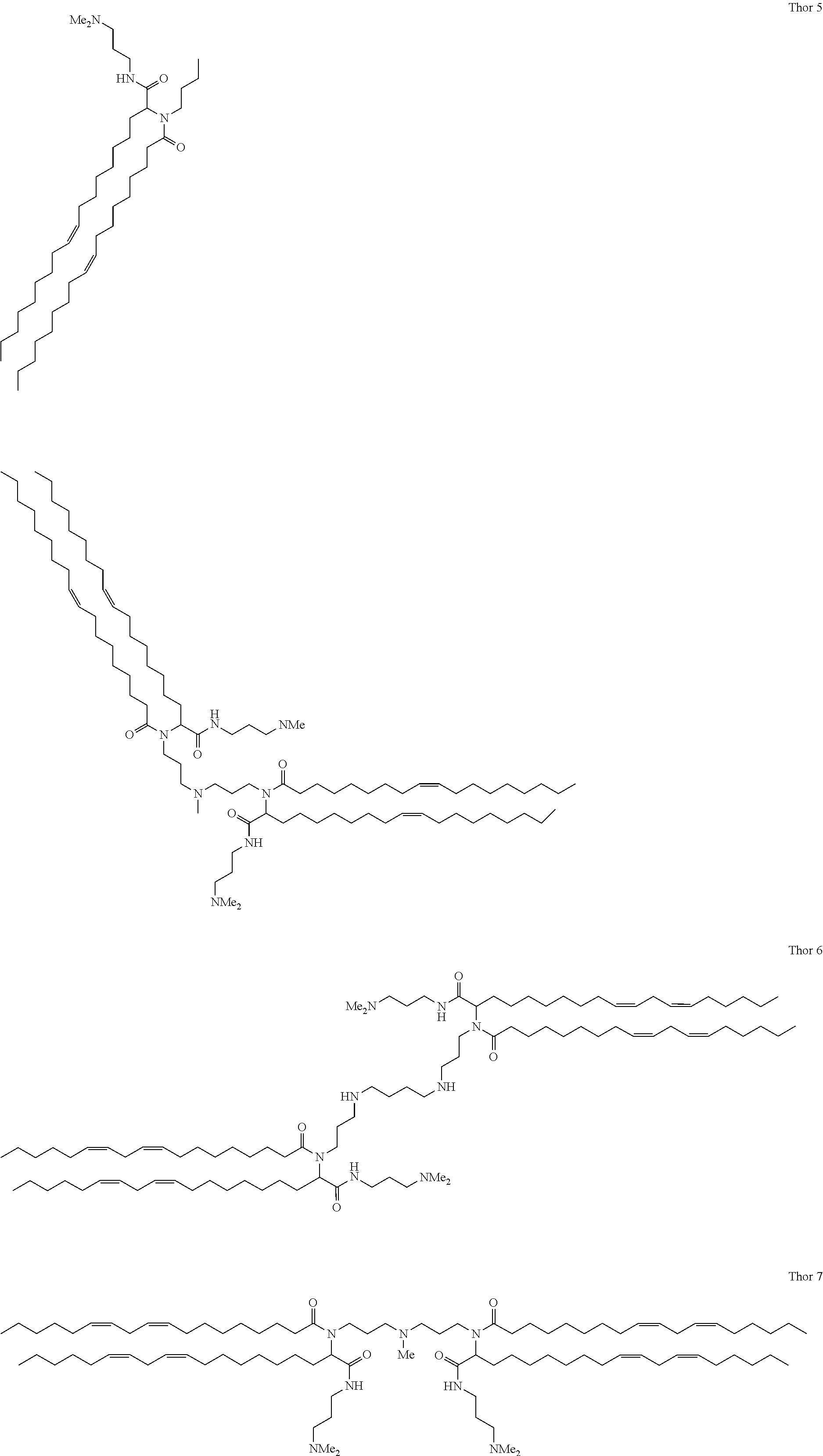

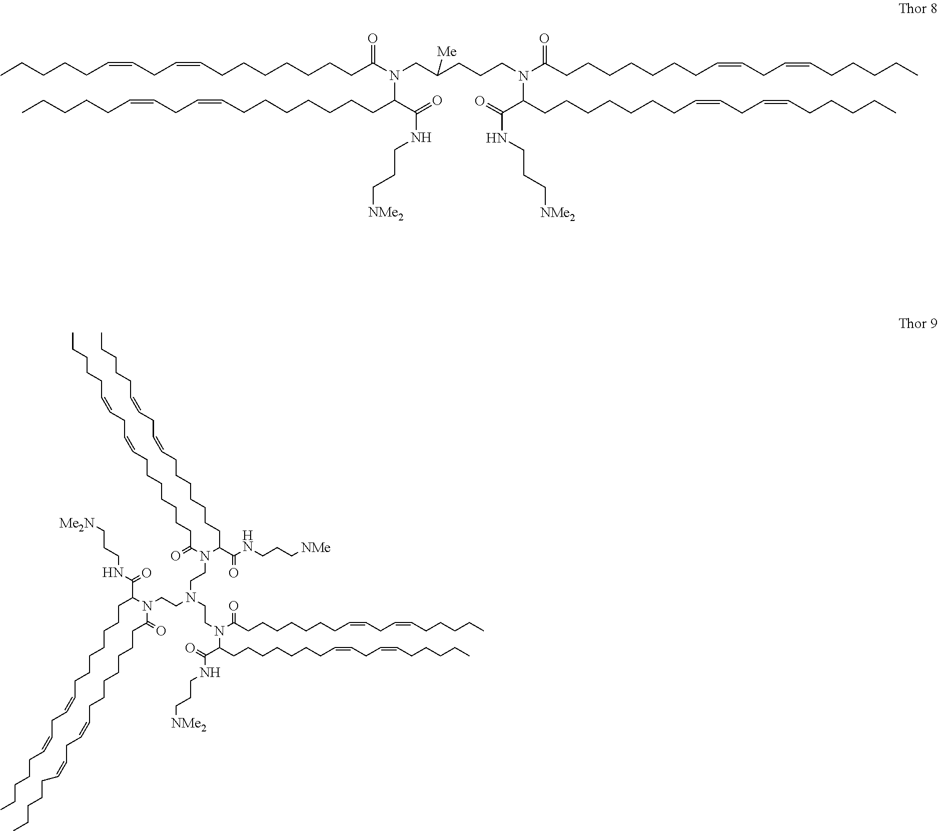

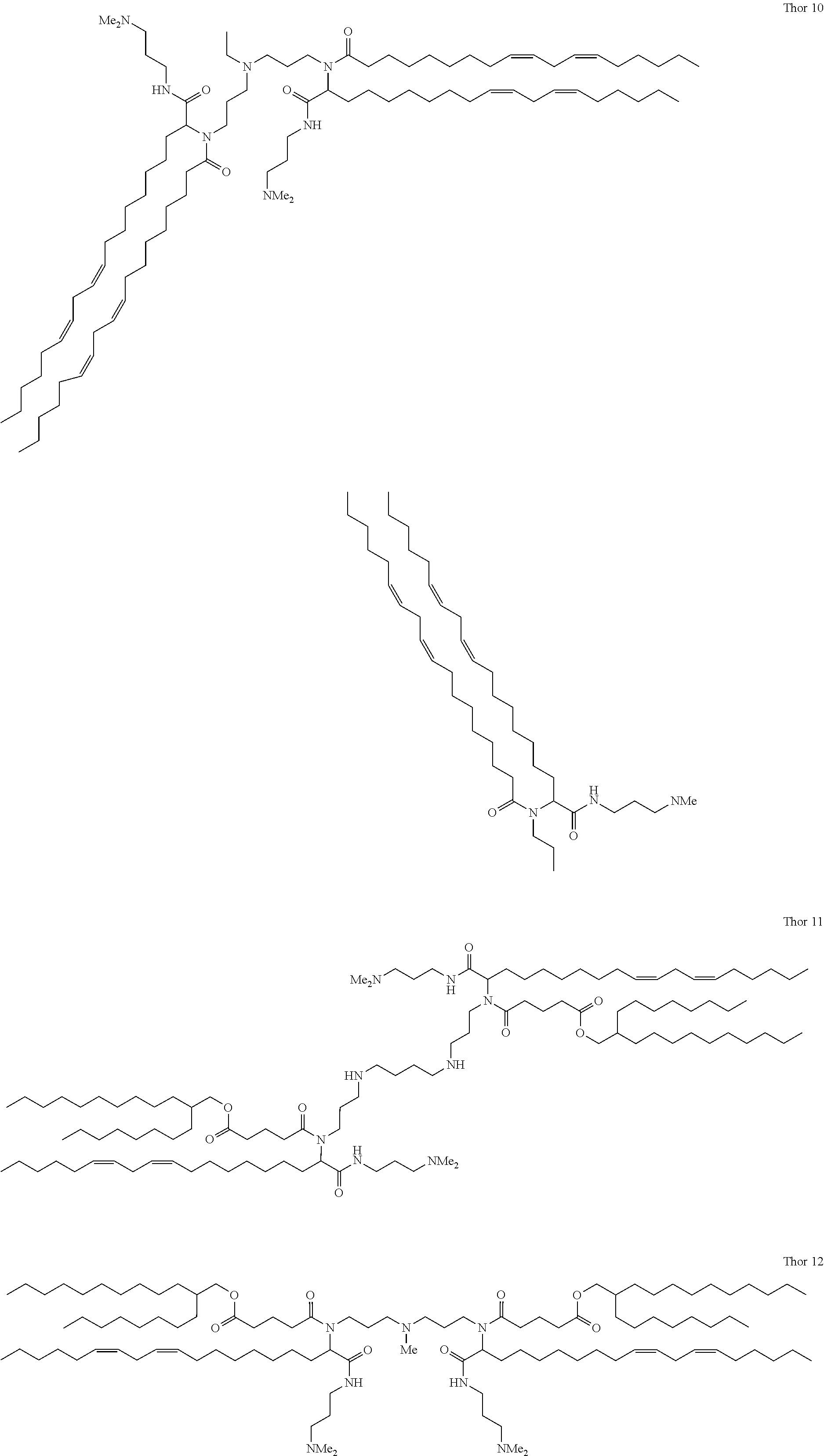

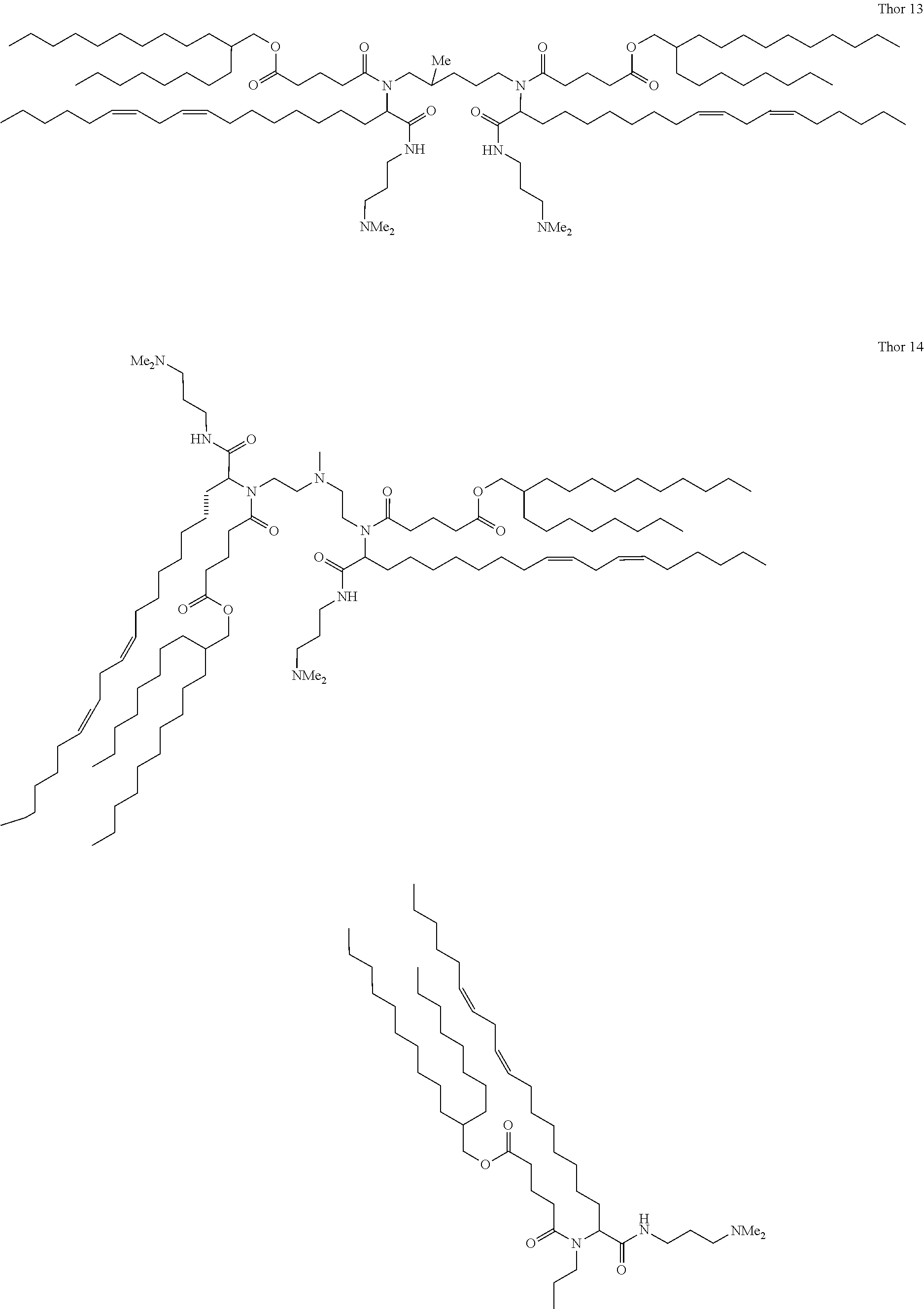

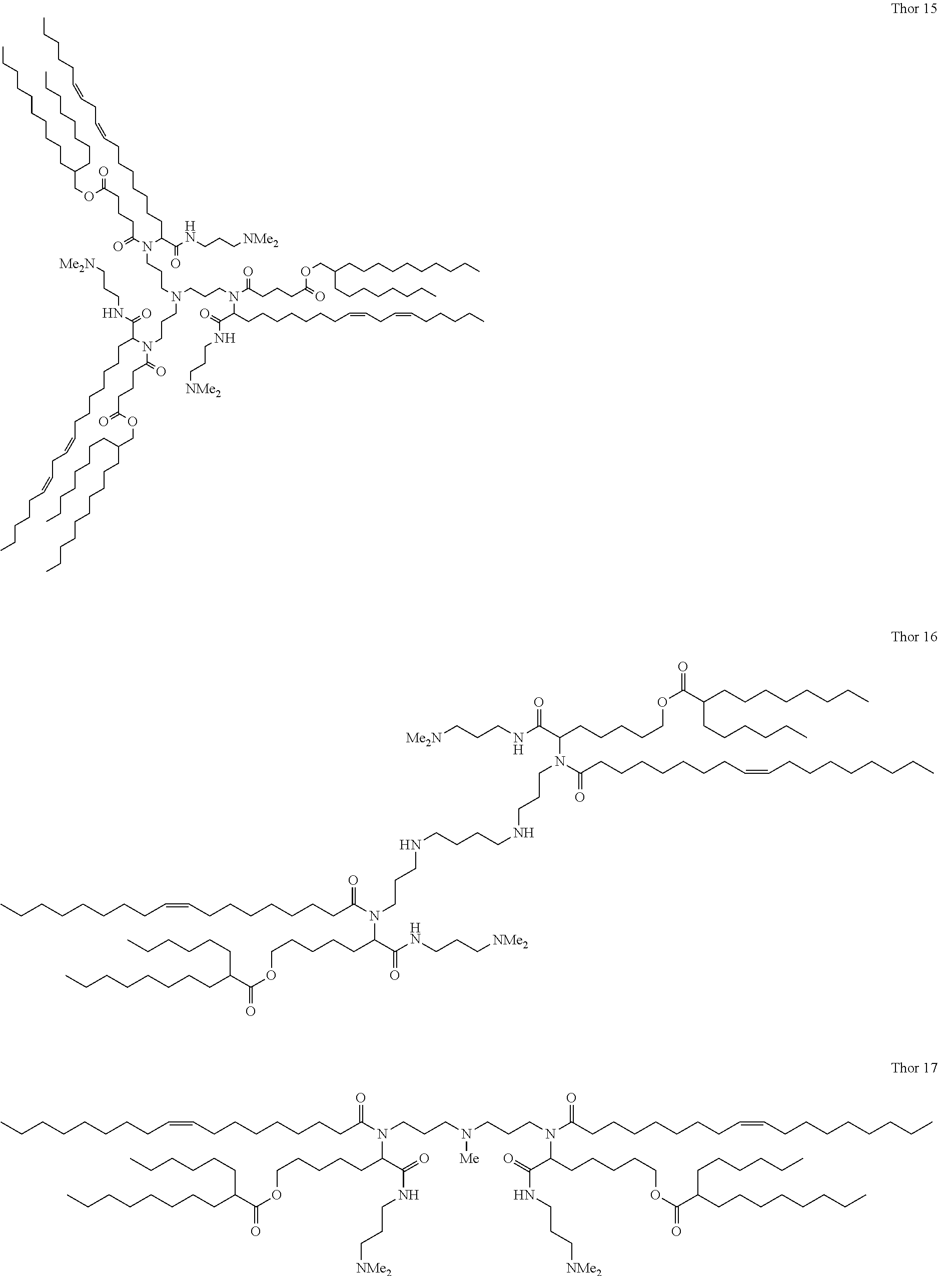

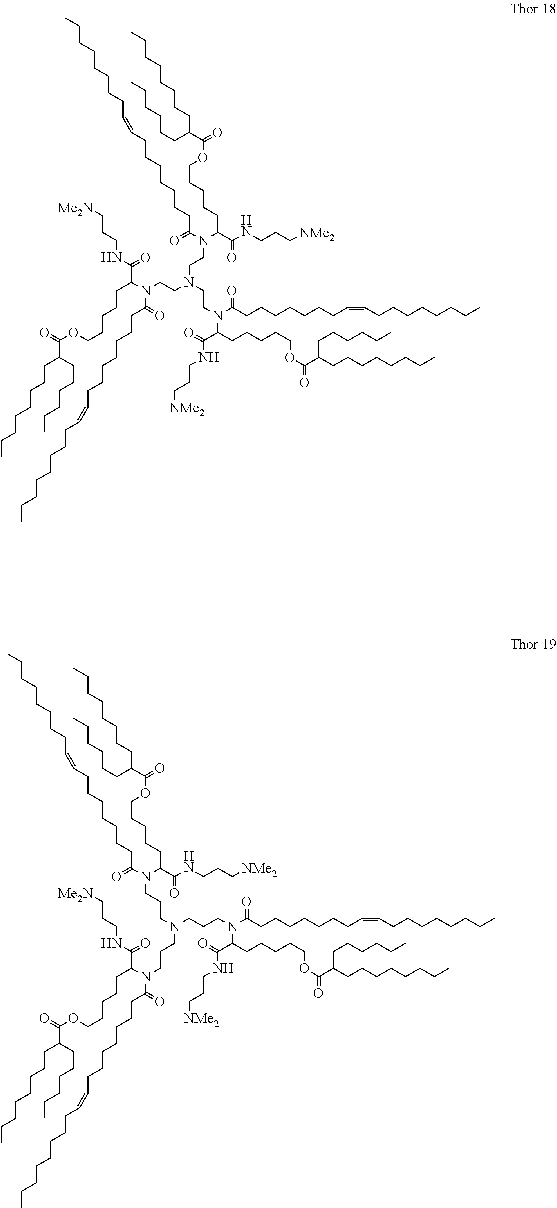 [SMILES: CCCCCCCC/C=C\CCCCCCCC(=O)N(CCN(CCN(C(=O)CCCCCCC/C=C\CCCCCCCC)C(CCCCCOC(=O)C(CCCCCC)CCCCCCCC)C(=O)NCCCN(C)C)CCN(C(=O)CCCCCCC/C=C\CCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C ; CCCCCCCC/C=C\CCCCCCCC(=O)N(CCCN(CCCN(C(=O)CCCCCCC/C=C\CCCCCCCC)C(CCCCCOC(=O)C(CCCCCC)CCCCCCCC)C(=O)NCCCN(C)C)CCCN(C(=O)CCCCCCC/C=C\CCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C]

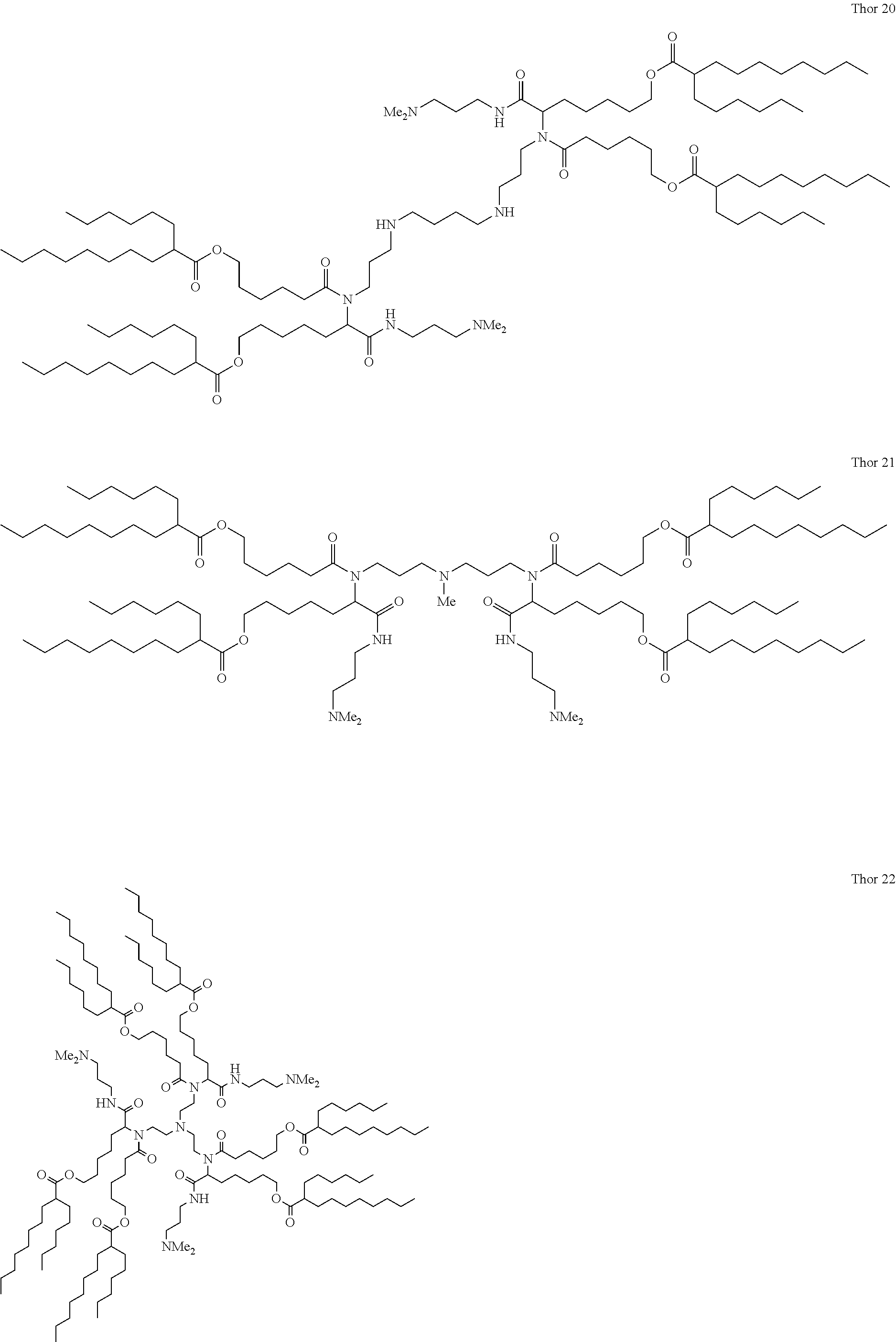

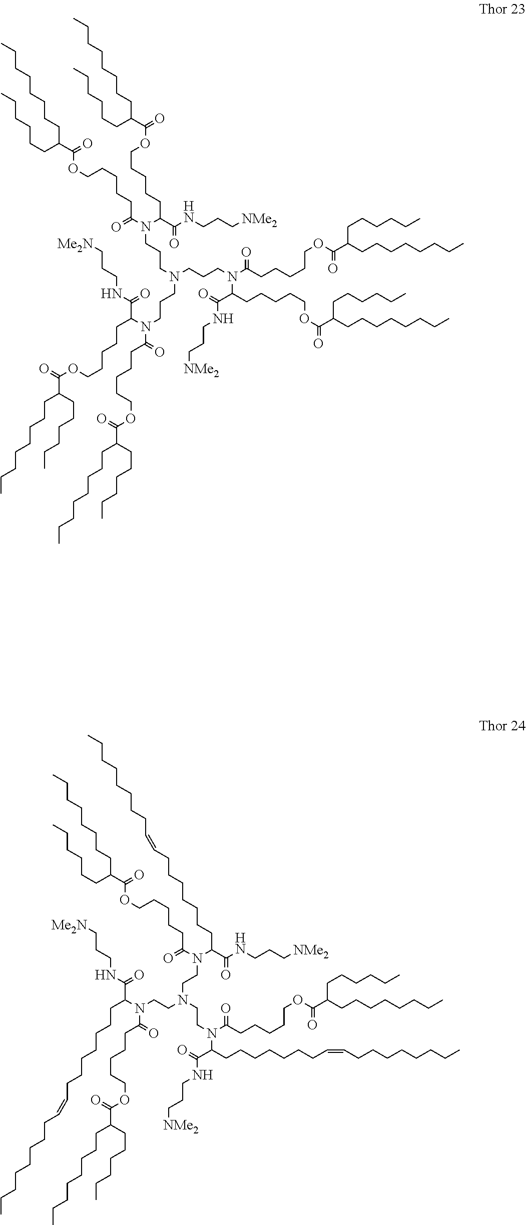 [SMILES: CCCCCCCCC(CCCCCC)C(=O)OCCCCCC(=O)N(CCCN(CCCN(C(=O)CCCCCOC(=O)C(CCCCCC)CCCCCCCC)C(CCCCCOC(=O)C(CCCCCC)CCCCCCCC)C(=O)NCCCN(C)C)CCCN(C(=O)CCCCCOC(=O)C(CCCCCC)CCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C ; CCCCCCCC/C=C\CCCCCCCC(C(=O)NCCCN(C)C)N(CCN(CCN(C(=O)CCCCCOC(=O)C(CCCCCC)CCCCCCCC)C(CCCCCCC/C=C\CCCCCCCC)C(=O)NCCCN(C)C)CCN(C(=O)CCCCCOC(=O)C(CCCCCC)CCCCCCCC)C(CCCCCCC/C=C\CCCCCCCC)C(=O)NCCCN(C)C)C(=O)CCCCCOC(=O)C(CCCCCC)CCCCCCCC]

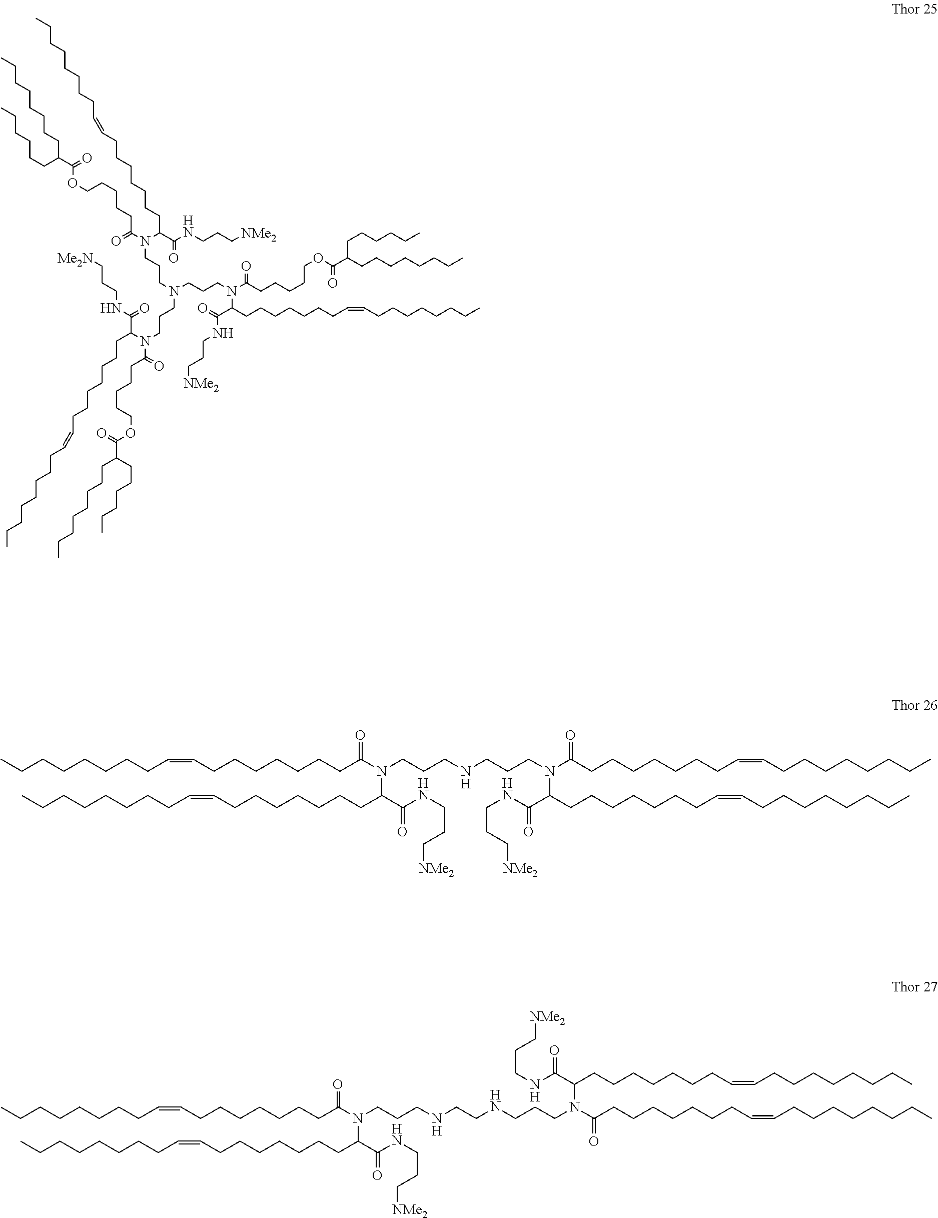

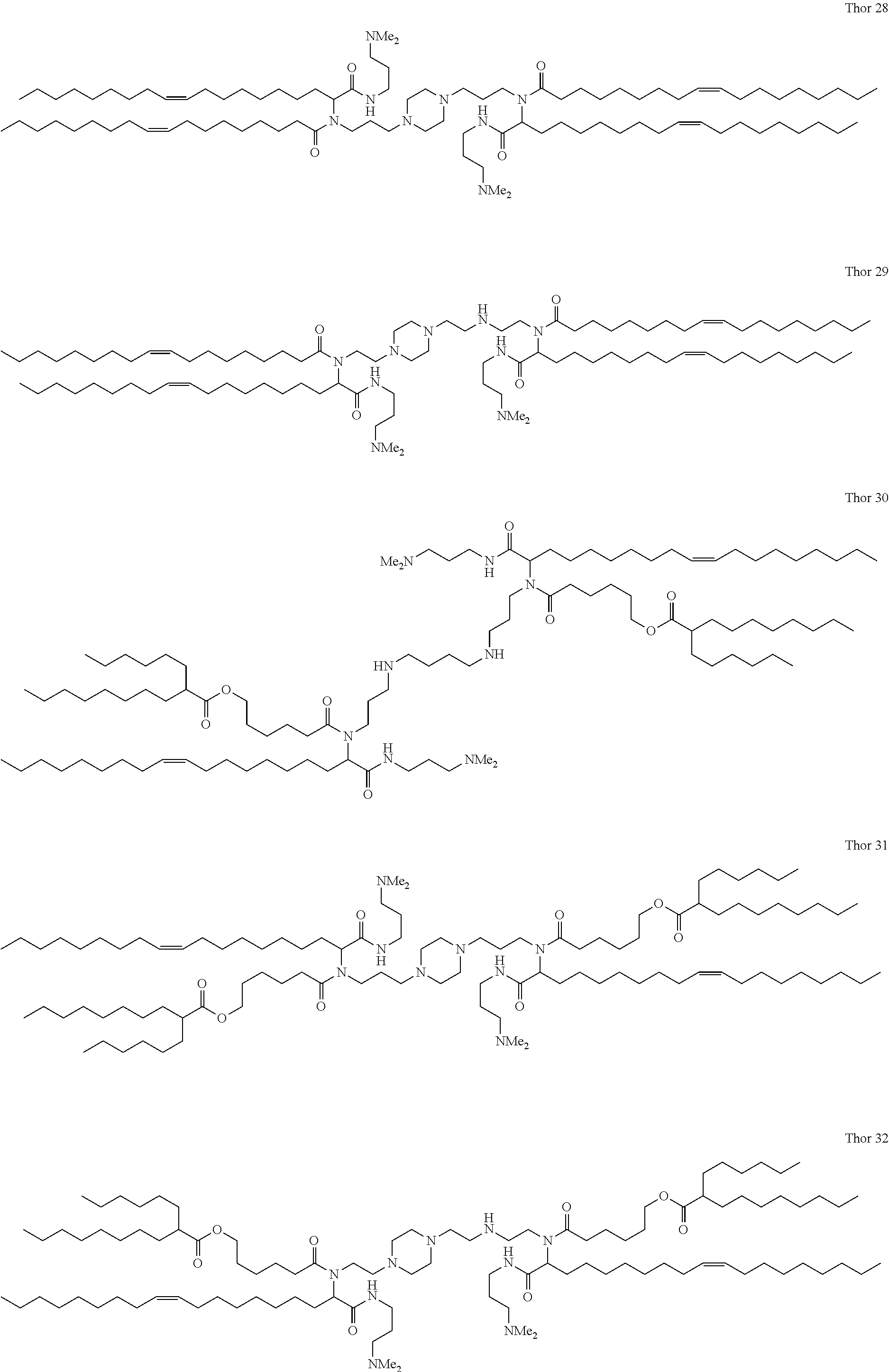

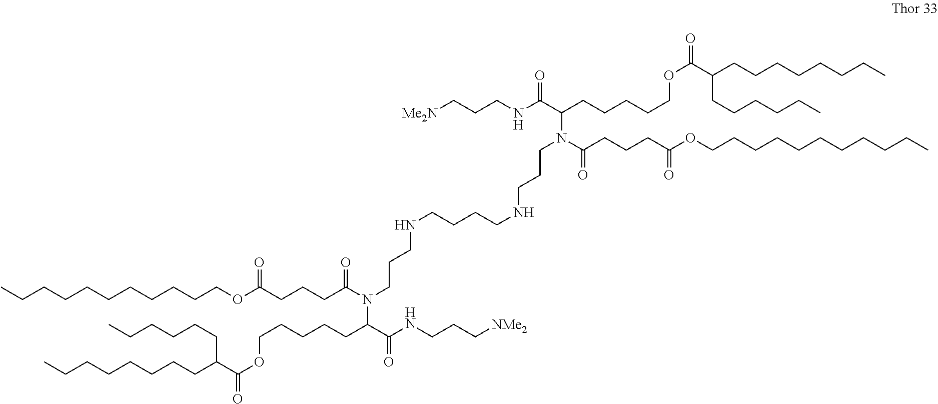 [SMILES: CCCCCCCCCCCOC(=O)CCCC(=O)N(CCCNCCCCNCCCN(C(=O)CCCC(=O)OCCCCCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C]

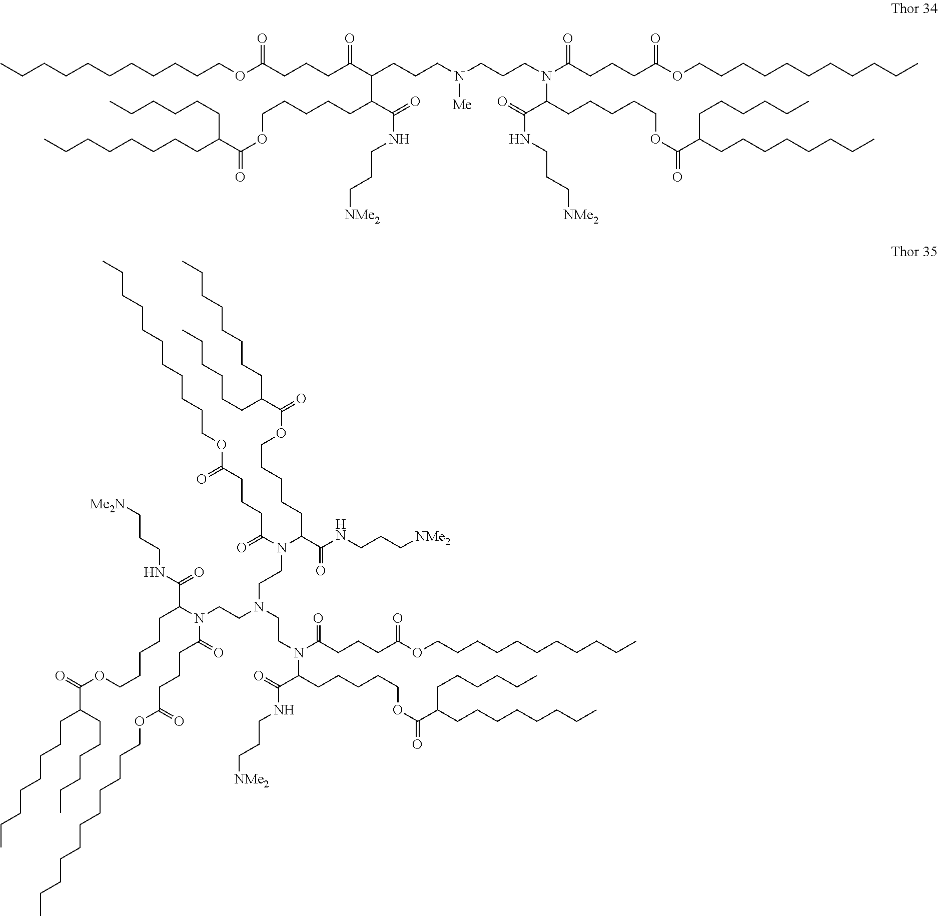 [SMILES: CCCCCCCCCCCOC(=O)CCCC(=O)C(CCCN(C)CCCN(C(=O)CCCC(=O)OCCCCCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C ; CCCCCCCCCCCOC(=O)CCCC(=O)N(CCN(CCN(C(=O)CCCC(=O)OCCCCCCCCCCC)C(CCCCCOC(=O)C(CCCCCC)CCCCCCCC)C(=O)NCCCN(C)C)CCN(C(=O)CCCC(=O)OCCCCCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C]

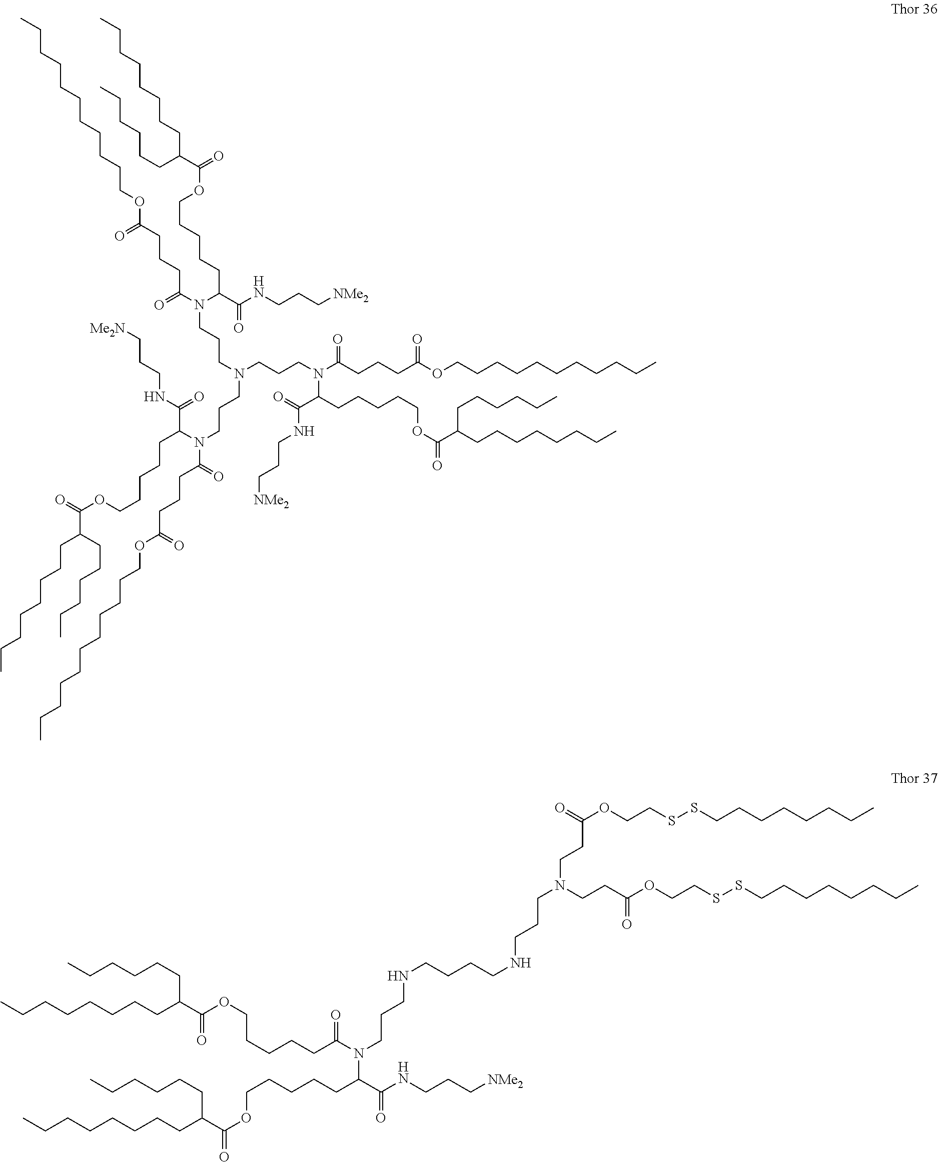

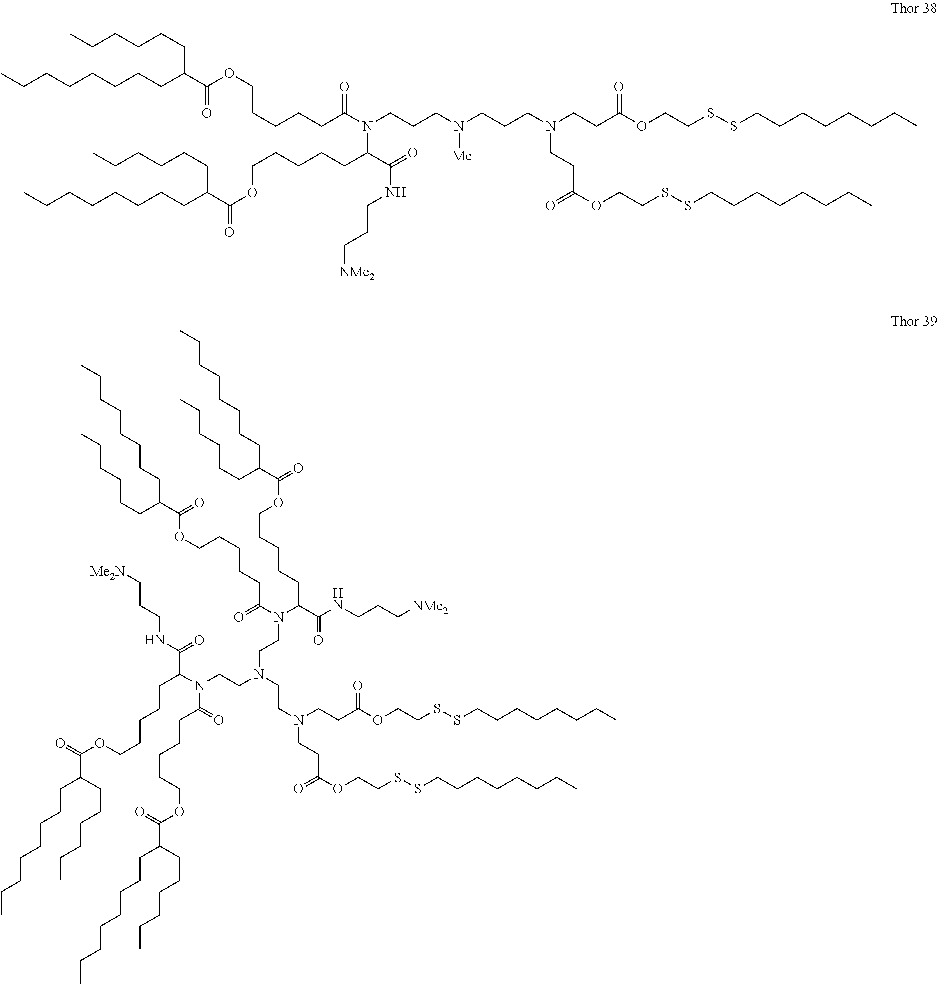

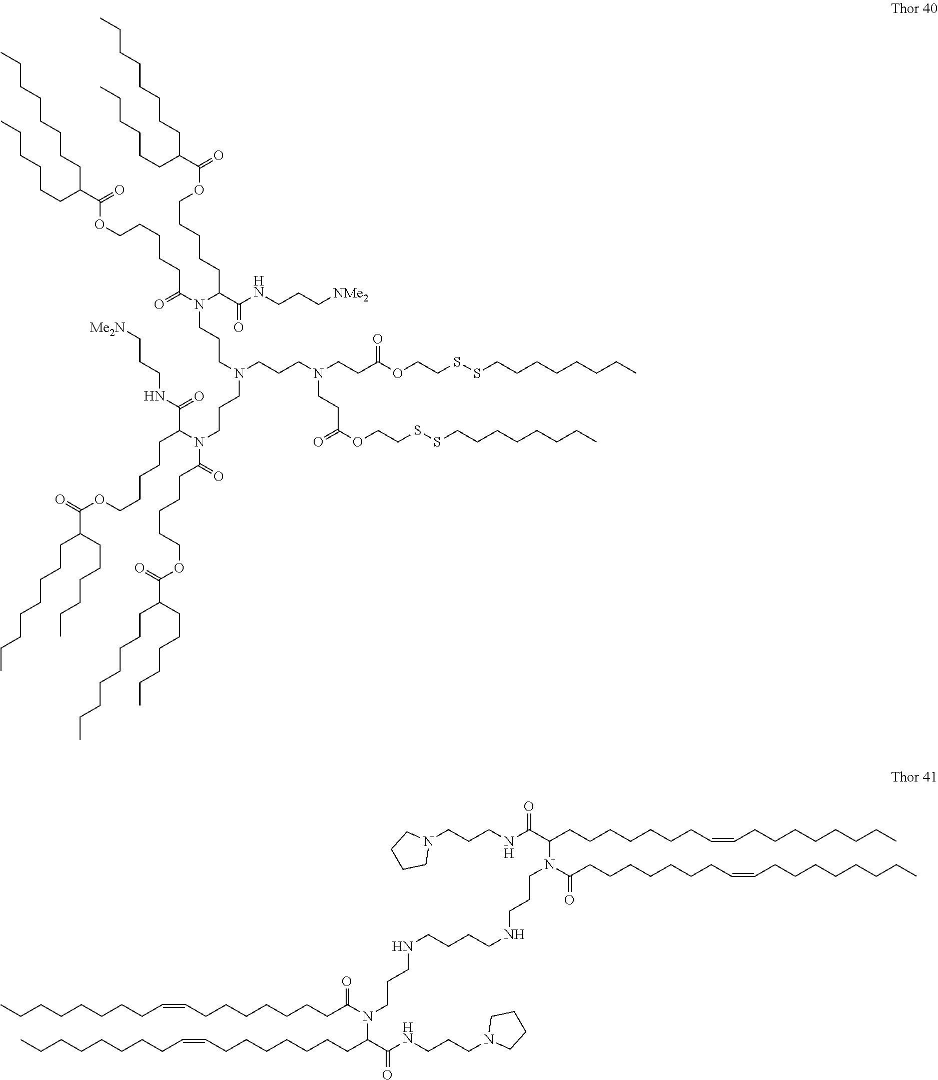

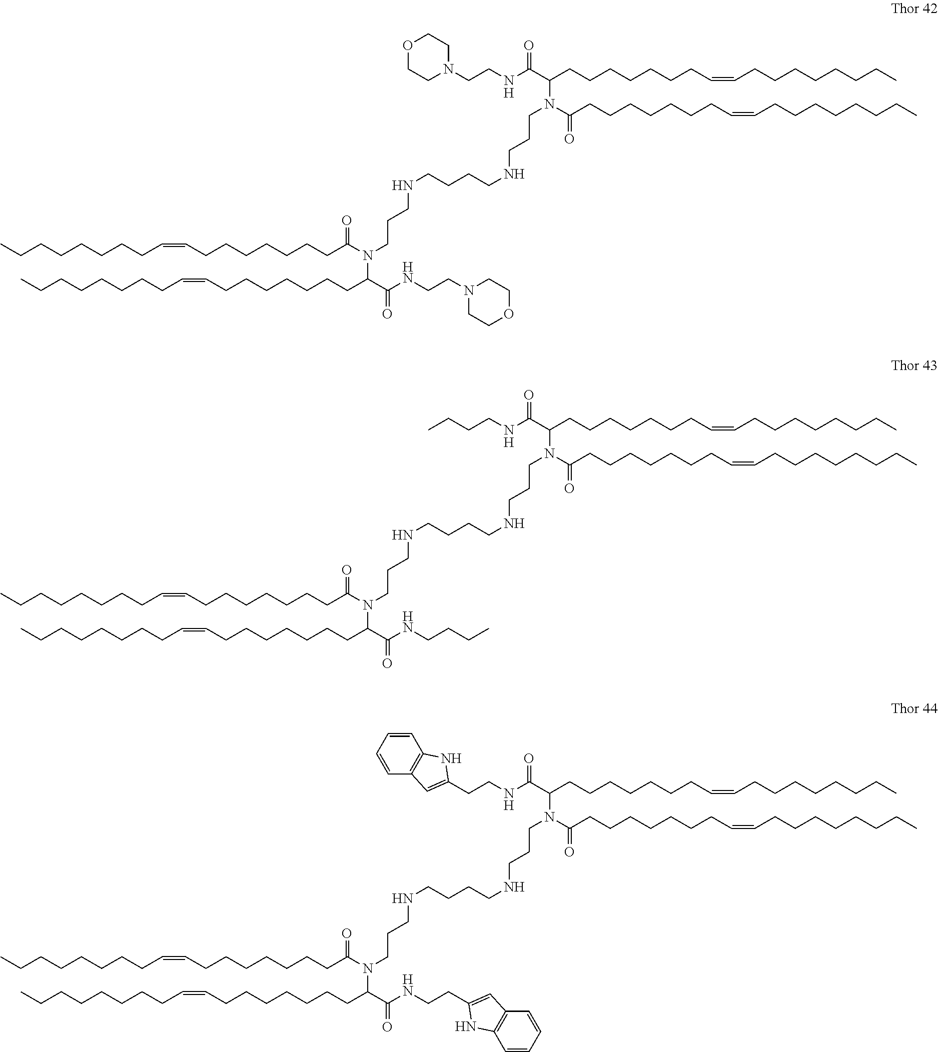

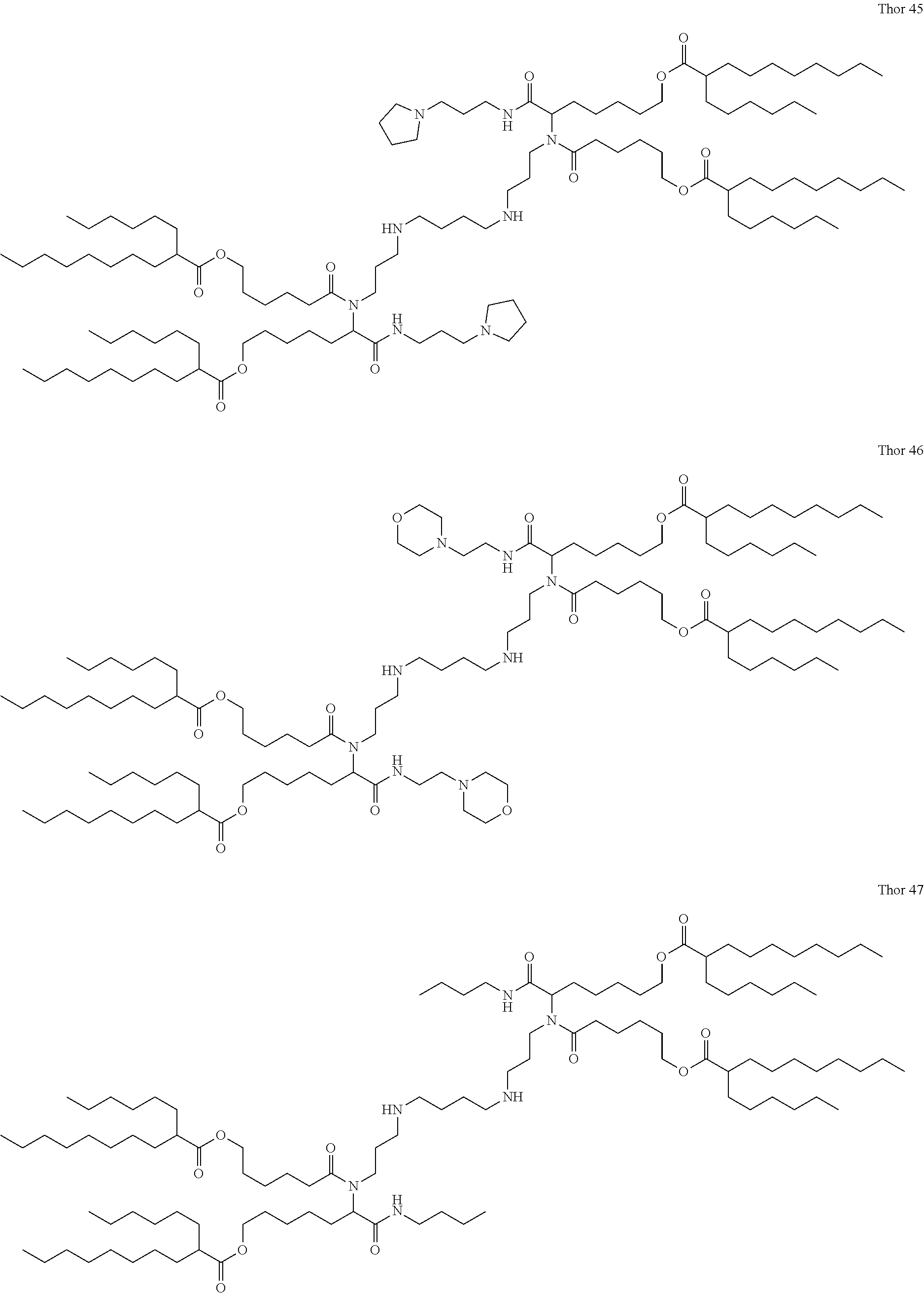

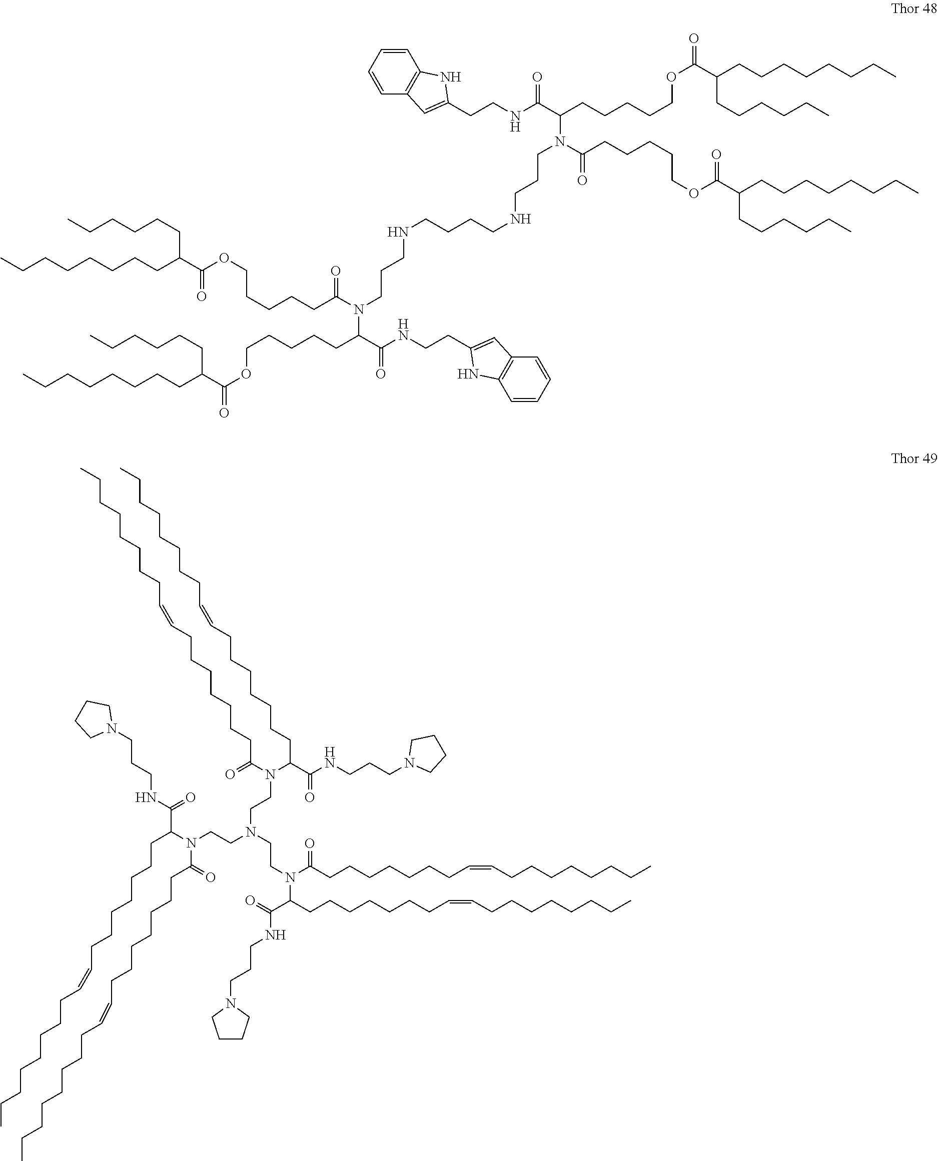 [SMILES: CCCCCCCCC(CCCCCC)C(=O)OCCCCCC(=O)N(CCCNCCCCNCCCN(C(=O)CCCCCOC(=O)C(CCCCCC)CCCCCCCC)C(CCCCCOC(=O)C(CCCCCC)CCCCCCCC)C(=O)NCCc1cc2ccccc2[nH]1)C(CCCCCOC(=O)C(CCCCCC)CCCCCCCC)C(=O)NCCc1cc2ccccc2[nH]1 ; CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCCN1CCCC1)CCN(C(=O)CCCCCCC/C=C\CCCCCCCC)C(CCCCCCC/C=C\CCCCCCCC)C(=O)NCCCN1CCCC1)C(CCCCCCC/C=C\CCCCCCCC)C(=O)NCCCN1CCCC1]

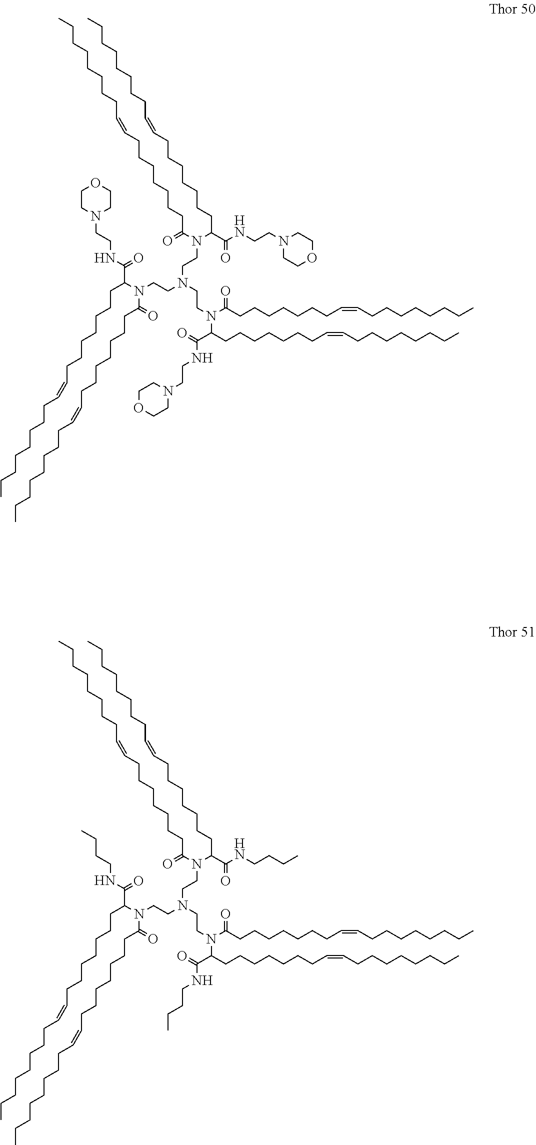 [SMILES: CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCN1CCOCC1)CCN(C(=O)CCCCCCC/C=C\CCCCCCCC)C(CCCCCCC/C=C\CCCCCCCC)C(=O)NCCN1CCOCC1)C(CCCCCCC/C=C\CCCCCCCC)C(=O)NCCN1CCOCC1 ; CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCCC)CCN(C(=O)CCCCCCC/C=C\CCCCCCCC)C(CCCCCCC/C=C\CCCCCCCC)C(=O)NCCCC)C(CCCCCCC/C=C\CCCCCCCC)C(=O)NCCCC]

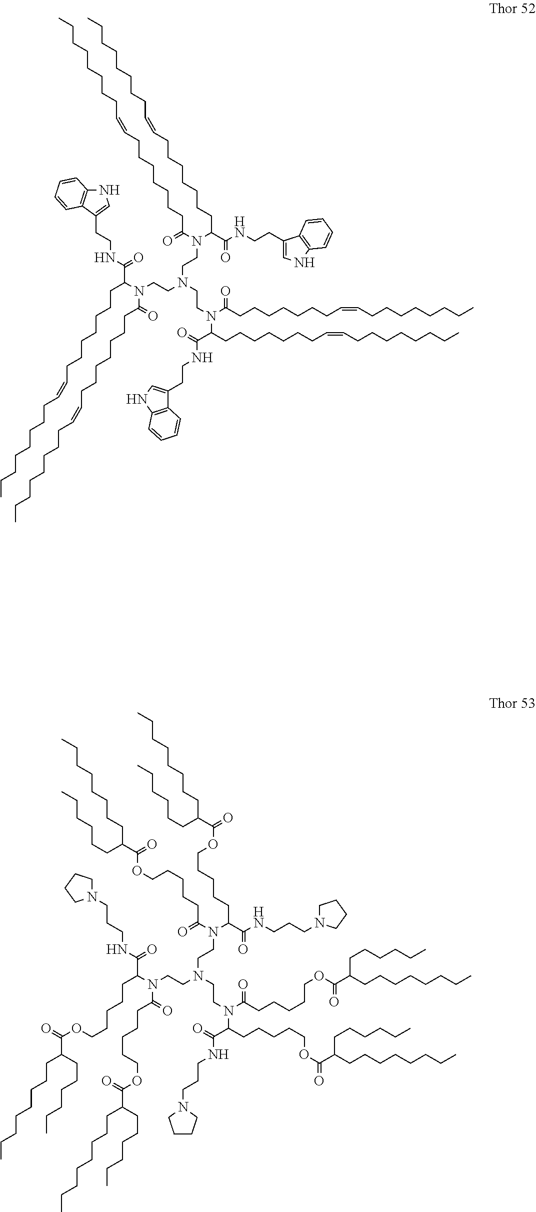 [SMILES: CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCc1c[nH]c2ccccc12)CCN(C(=O)CCCCCCC/C=C\CCCCCCCC)C(CCCCCCC/C=C\CCCCCCCC)C(=O)NCCc1c[nH]c2ccccc12)C(CCCCCCC/C=C\CCCCCCCC)C(=O)NCCc1c[nH]c2ccccc12 ; CCCCCCCCC(CCCCCC)C(=O)OCCCCCC(=O)N(CCN(CCN(C(=O)CCCCCOC(=O)C(CCCCCC)CCCCCCCC)C(CCCCCOC(=O)C(CCCCCC)CCCCCCCC)C(=O)NCCCN1CCCC1)CCN(C(=O)CCCCCOC(=O)C(CCCCCC)CCCCCCCC)C(CCCCCOC(=O)C(CCCCCC)CCCCCCCC)C(=O)NCCCN1CCCC1)C(CCCCCOC(=O)C(CCCCCC)CCCCCCCC)C(=O)NCCCN1CCCC1]

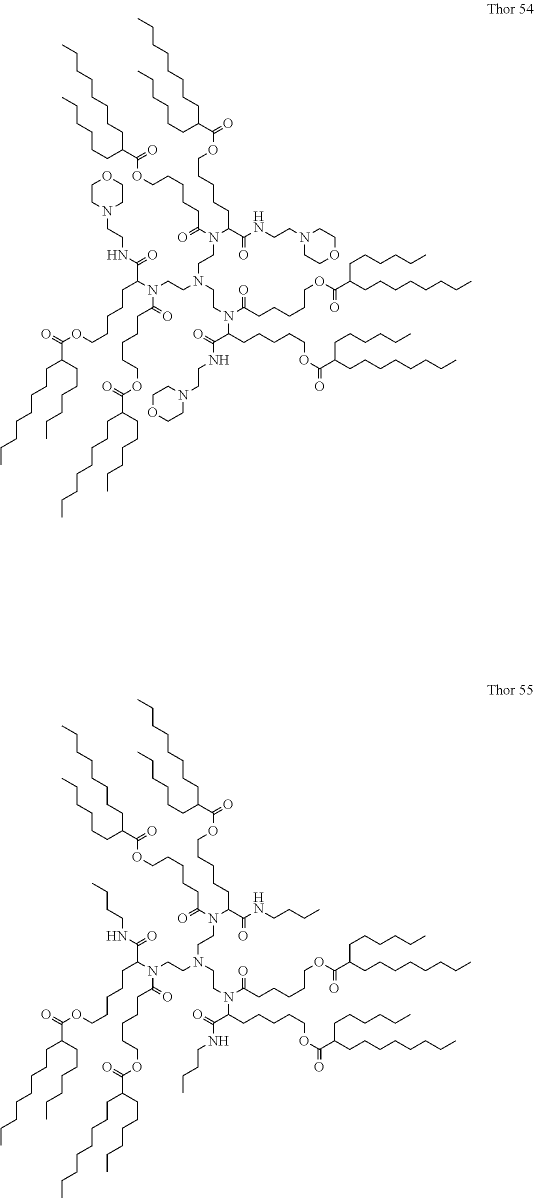

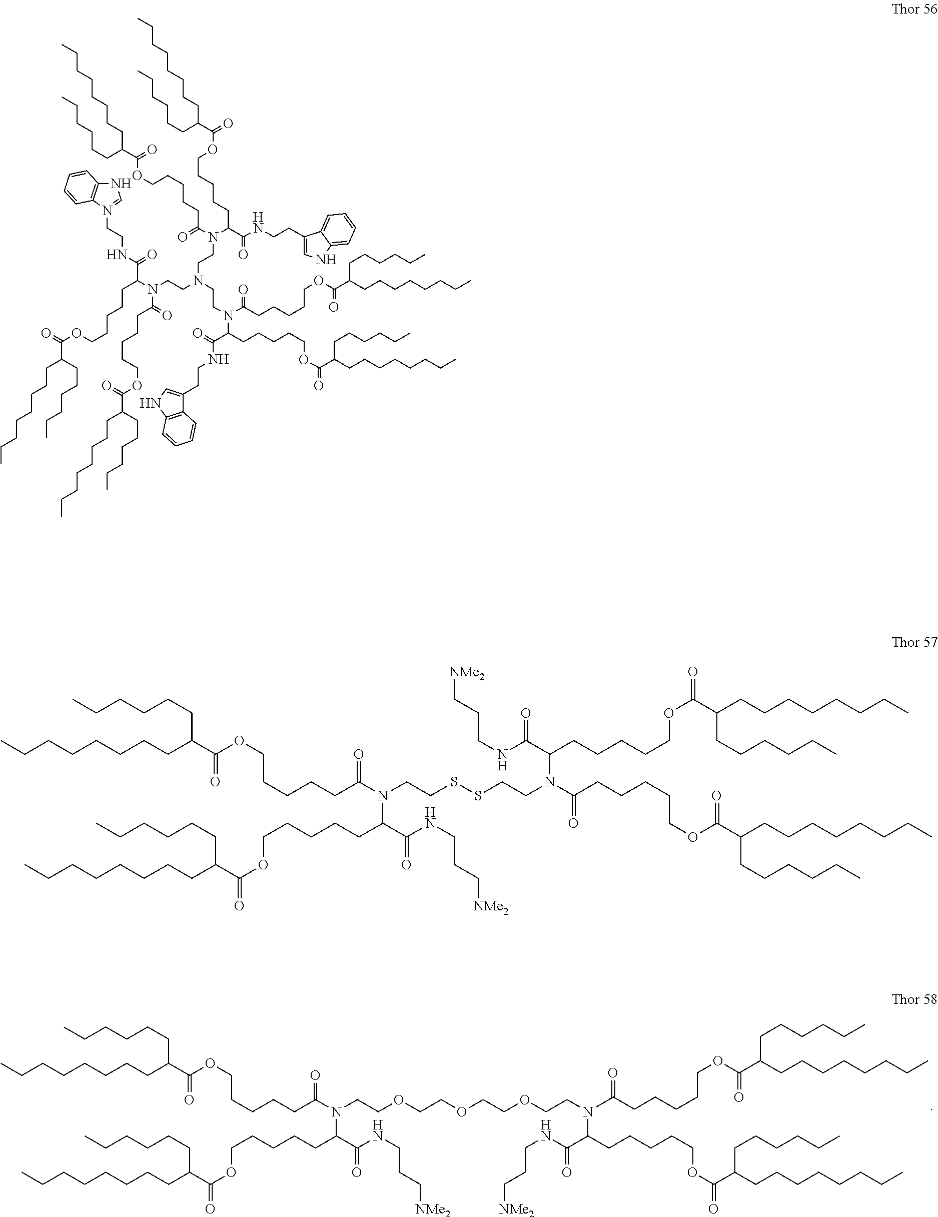

### III. Synthesis

The disclosed compounds are prepared from an isonitrile, an amine, a carboxylic acid and an aldehyde.

The isonitrile compound may be purchased from a commercial supplier or prepared as exemplified below, and as will be understood by a person of ordinary skill in the art of organic synthesis. An exemplary synthesis for an isonitrile compound proceeds as shown in Scheme 1.

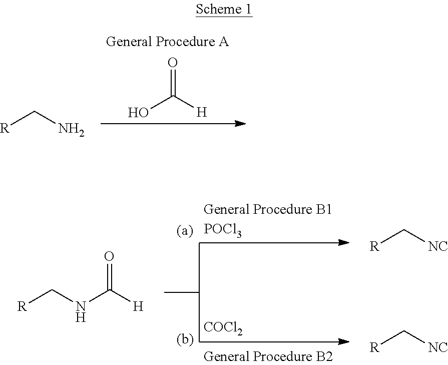

With respect to Scheme 1, the amine compound is treated with formic acid to form a formamide. The reaction proceeds in a suitable solvent, such as an aprotic solvent, for example, toluene, cyclohexane, acetonitrile, dioxane, or a combination thereof. The reaction may be heated, such as to reflux or from 100° C. to 150° C. to facilitate the reaction proceeding towards completion.

Following the heating step, the formamide compound is isolated, and optionally purified, such as by chromatography or vacuum distillation.

The formamide compound then is dehydrated to form the isonitrile. The dehydration reaction may be performed in the presence of a suitable reagent, such as, but not limited to, POCl₃ or phosgene, and also in the presence of a suitable base, such as an organic base, for example, a trialkylamine base such as triethylamine.

The aldehyde compound may be purchased from a commercial supplier or prepared as exemplified below, and as will be understood by a person of ordinary skill in the art of organic synthesis. An exemplary synthesis for an aldehyde compound proceeds as shown in Scheme 2.

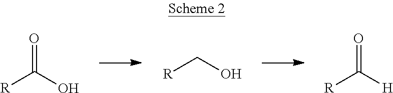

With respect to Scheme 2, a carboxylic acid compound is treated with a suitable reducing agent to form an alcohol. The reducing agent may be any reducing agent suitable to form the aldehyde, such as, but not limited to, an aluminum hydride reagent such as lithium aluminum hydride or diisobutylaluminium hydride, or a borohydride reagent such as sodium borohydride. The reaction is performed in a suitable solvent, such as THF or ether.

The alcohol then is oxidized to form the aldehyde. Suitable oxidizing agents include, but are not limited to Dess-Martin periodinane, pyridinium chlorochromate, or pyridinium dichromate. The reaction is performed in a suitable solvent, such as THF, ether, or a chlorinated solvent, such as dichloromethane.

The disclosed compounds may be prepared from an aldehyde, an acid, an isonitrile and an amine compound. Typically, one equivalent of the aldehyde, acid and isonitrile is used for each primary amine group on the amine compound. An exemplary synthesis proceeds as shown in Scheme 3.

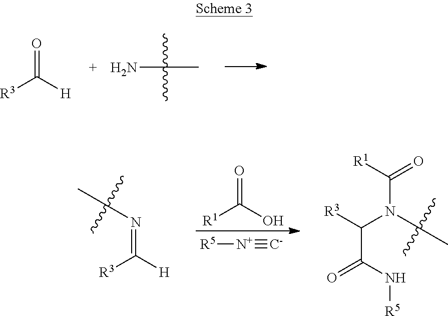

With respect to Scheme 3, the aldehyde and amine compounds are combined in a suitable solvent to form the Schiff base intermediate. The solvent may be any solvent suitable to facilitate the Schiff base formation, such as, but not limited to, an alcohol (for example, an alkyl alcohol such as methanol, ethanol, isopropanol or a combination thereof), or an aprotic solvent such as toluene or a chlorinated solvent, optionally with removal of water. The reaction may be agitated by any suitable technique, such as, but not limited to, stirring, shaking, or sonication.

Subsequently, the acid and isonitrile are added to the Schiff base intermediate and the reaction mixture may be agitated for a time period suitable to form the disclosed compound. The reaction may proceed for from 12 hours or less to 28 hours or more, such as 18 hours to 30 hours, or about 24 hours.

Exemplary isonitrile compounds suitable for use in the disclosed synthesis include, but are not limited to:

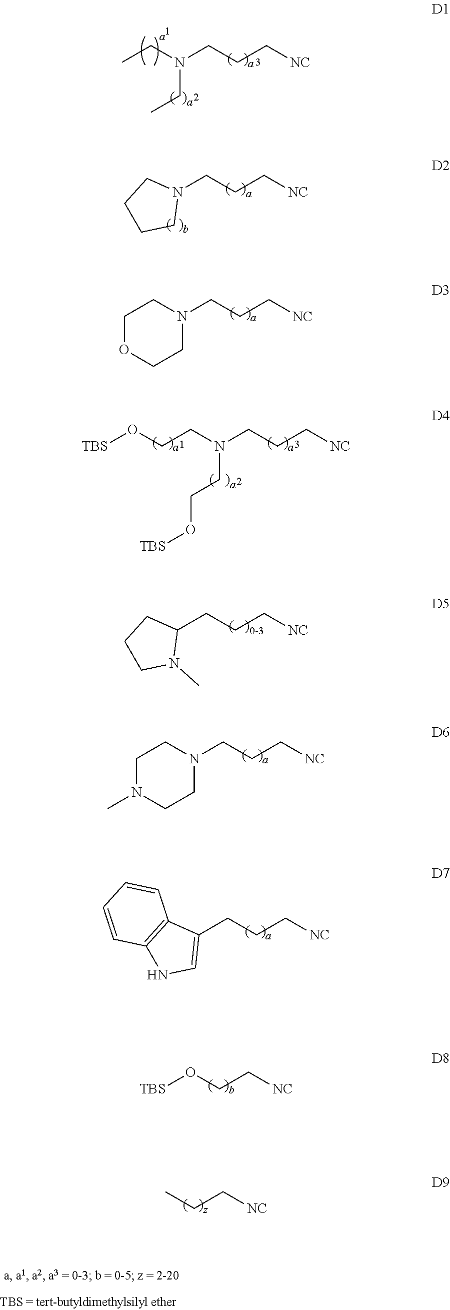

Exemplary aldehyde compounds suitable for use in the disclosed synthesis include, but are not limited to:

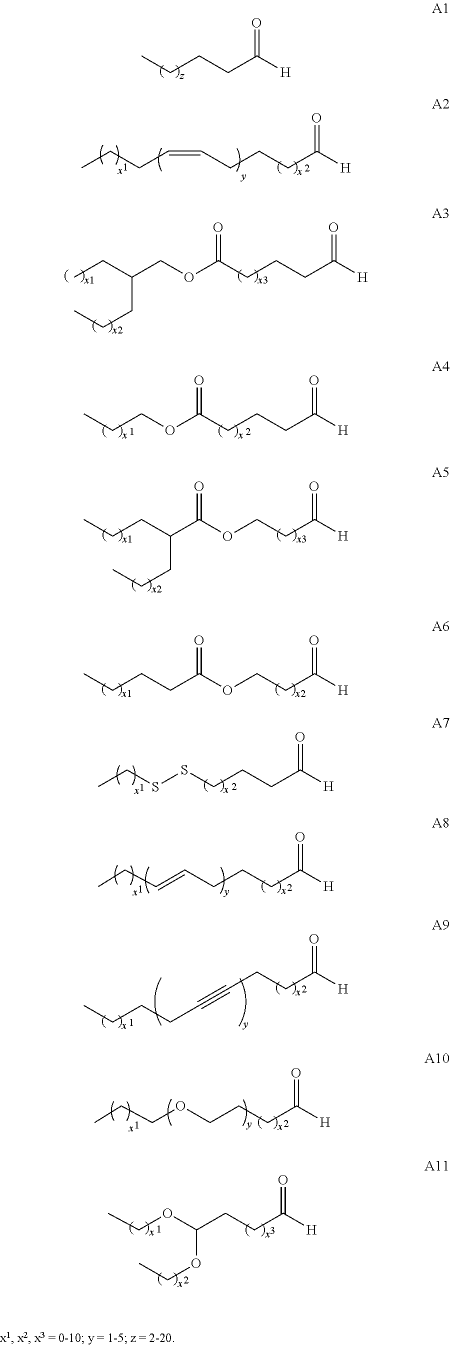

Exemplary acid compounds suitable or use in the disclosed synthesis include, but are not limited to:

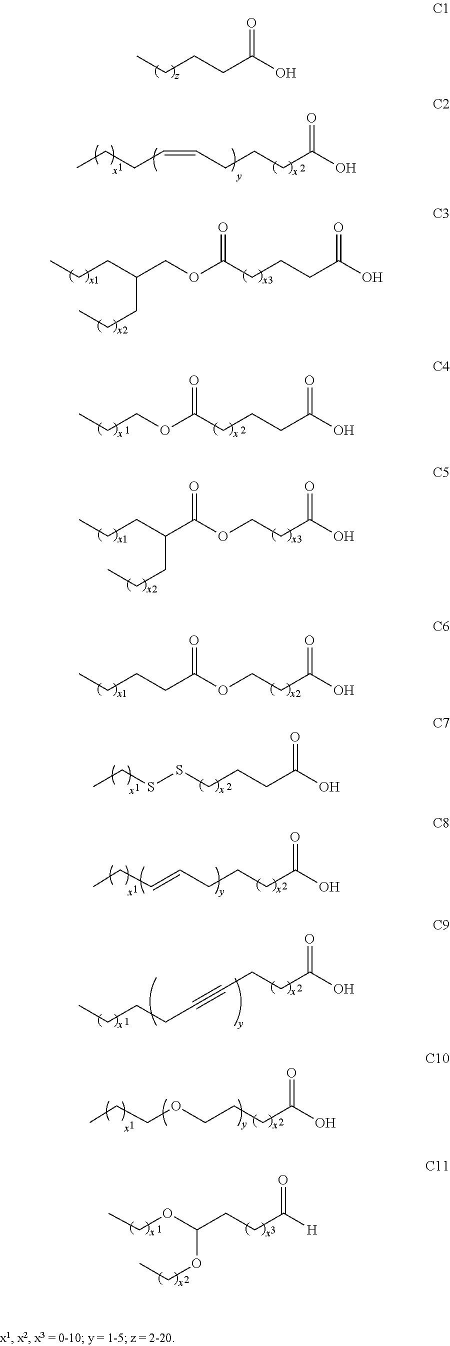

Exemplary amine compounds suitable for use in the disclosed synthesis include, but are not limited to:

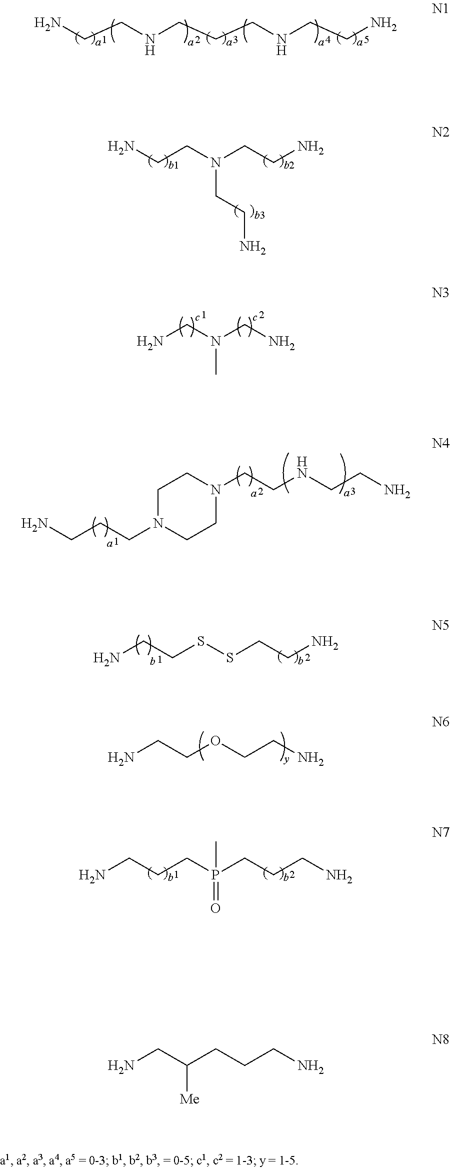

### IV. Applications

The disclosed compounds are useful for facilitating the delivery of molecules, such as nucleic acids, peptides, polypeptides, and/or small molecules, into cells, enabling numerous applications in research and clinical settings. The disclosed compounds typically possess several ionizable moieties and exhibit characteristics from classic lipids to lipidoids. Unlike currently known ionizable lipids, which have a strong avidity to be taken up by the liver, the disclosed compounds primarily transfect in the lung and, to a lesser extent, the spleen. The ability to achieve targeted transfection in specific organs holds immense potential for various applications, such as gene therapy and targeted drug delivery.

In some aspects, the disclosed compounds are useful to make lipid nanoparticle compositions that are useful for delivering molecules, such as therapeutic or prophylactic agents. Lipid nanoparticle (LNP) compositions may include one or more lipid components and one or more agents, such as a nucleic acid molecule, that may be associated and/or encapsulated by the lipid components. A nanoparticle composition may be designed for one or more specific applications, targets, and/or diseases. The elements of a nanoparticle composition may be selected based on a particular application or target and/or based on the efficacy, toxicity, expense, ease of use, availability, synthetic pathways, or other properties. In some aspects, the lipid nanoparticles comprise five components: 1) and agent, such as a nucleic acid (e.g., mRNA), 2) ionizable lipid (e.g., a lipid disclosed herein), 3) phospholipid, 4) structural lipid, and 5) polymer-conjugated lipid. Exemplary phospholipids, structural lipids, and polymer-conjugated lipids suitable for use in the disclosed lipid nanoparticle compositions are disclosed herein. The chemical structures and quantities of comprising lipids and other constituents may influence gene delivery efficacy, particle stability, and toxicity to the cells, and may be adjusted depending on the applications or targets. In general, the quantities of the lipids may be described by the molar ratios between the comprising components.

### A. Nanoparticle Preparation

Various mixing methods and protocols may be used to produce the formulations. Specifically, lipid nanoparticles can be made by any suitable mixing processes such as, but not limited to, pipet mixing, syringe mixing, T-junction mixing, or microfluidic mixing of two or more solutions and/or suspensions, one of which contains one or more nucleic acids and the other has lipid components. Additional chemical compounds may be introduced in either or both of the fluid volumes or streams depending on their solubility. In certain aspects, the nucleic acid solution includes an aqueous buffer, such as a citrate or acetate buffer, typically 5-100 mM, to maintain the solution at acidic pH, such as at a pH of from less than 7, for example, 2, 3, 4, 5, or 6. In certain aspects, the lipids are dissolved in alcohol, such as ethanol.

The volumes of the fluids, the flow rate of the fluid streams, may be adjusted or optimized depending on the amount of the agent, such as a nucleic acid, target nanoparticle size, polydispersity, encapsulation efficiency, and other features. A volumetric ratio between nucleic acid solution and lipid solution may vary from about 1:1 to 20:1, such as 2:1, 3:1. 5:1, 10:1, 15:1, 20:1 or as necessary. In some aspects, the total flow rate may be from about 5 ml/min to 50 ml/min.

The lipid solution contains different lipid components including ionizable lipids, phospholipids, structural lipids and polymer conjugated lipids. The amount of ionizable lipid used in the lipid nanoparticle (LNP) formulation ranges from 20 mol % or less to 100 mol % of the total amount of lipids in the nanoparticle, such as from 20% w/w or less to 100% w/w ratio of nucleic acid to be encapsulated.

In certain aspects, after addition, the mixture is isolated, or purified from unincorporated components. The LNPs may be directly buffer exchanged by using physiological buffer at a 1:1 to 1:10 v/v dilution ratio or they can be dialyzed once or multiple times at 4° C. or room temperature, if needed, using phosphate-buffered saline. After the purification is complete, the nanoparticles may be concentrated, for example, in a centrifuge.

### Phospholipids

Typically, the amount of phospholipid used in the lipid nanoparticle is from 0 to 30 mol % of the total lipids, such as from greater than zero to 30 mol %. The phospholipid may be any phospholipid suitable to form the nanoparticles, such as, but not limited to, 1,2-dioleoyl-sn-glycero-3-phosphoethanolamine (DOPE), 1,2-distearoyl-sn-glycero-3-phosphocholine (DSPC)1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, and/or lysophosphatidylethanolamine (LPE).

### Structural Lipids

The amount of structural lipid used is from 0 to 70 mol % of the total amount of lipids, such as from greater than zero to 70 mol %. The structural lipid used is any suitable structural lipid, such as, but not limited to, cholesterol, beta-sitosterol, cholestanol, fucosterol, campesterol, stigmastanol, brassicasterol, ergosterol, and/or stigmasterol.

### Polymer-Conjugated Lipids

The amount of polymer-conjugated lipid (PEG-lipid) used is from 0 to 10 mol % of the total amount of lipids, such as from greater than zero to 10 mol %. The PEG lipid is any suitable PEG lipid, such as, but not limited to, PEG-modified phosphatidylethanolamines, PEG-modified phosphatidic acids, PEG-modified phosphoethanolamine, PEG-modified ceramides (PEG-CER), PEG-modified dialkylamines, PEG-modified diacylglycerols (PEG-DAG), PEG-modified dialkylglycerols, and mixtures thereof. For example, a PEG lipid may be 14:0 PEG2000 PE, PEG-c-DOMG, PEG-DMG, PEG-DLPE, PEG-DMPE, PEG-DPPC, a PEG-DSPE lipid, or PEG-lipid derivatives, such as, but not limited to that contain carboxylic acid, maleimide, amine, azides, thiols or active esters at the PEG terminal. The molecular weight of PEG-lipid ranges from 200-40,000 daltons or more.

### B. Properties

The mean size of a nanoparticle composition may be from 10's of nm to 100's of nm. In some aspects, the nanoparticle composition have an average diameter of from 50 nm to 200 nm, such as from 75 nm to 175 nm, as determined by dynamic light scattering. For non-spherical nanoparticles, the diameter is determined as the largest (longest) dimension of the nanoparticle. Formulations of 200 nm or less typically are relevant to physical and biochemical targeting through intravenous injections. Nanoparticles larger than 200 nm often activate the complement system and so are rapidly eliminated from systemic circulation.

A nanoparticle composition disclosed herein may be relatively homogenous as indicated by a polydispersity index (PDI) which may vary from 0 to 1, such as from greater than zero to 0.7, from greater than zero to 0.5, from greater than zero to 0.4, from greater than zero to 0.3, or from greater than zero to 0.2. A lower PDI indicates a narrower particle size range which may be preferrable in some aspects. In certain aspects, the nanoparticle composition has a PDI of from greater than zero to 0.3 or from greater than zero to 0.2. The PDI of the disclosed formulations is determined by dynamic light scattering.

Zeta potential of a nanoparticle composition may be used to indicate electrokinetic potentials of the composition. For example, the zeta potential may describe the surface charge of a nanoparticle composition and can indicate whether the particle is anionic, cationic, or neutral. In some aspects, the formulations disclosed herein were positively charged to neutral and had a zeta potential of +15 mV or less, such as from less than +15 mV to 0 mV (neutral charge).

In some aspects, the disclosed formulations exhibited high encapsulation efficiency of nucleic acids such as mRNA. In some aspects, the encapsulation efficiency of the disclosed formulations is greater than 80%, such as 85% or more, or 90% or more. Nucleic acid encapsulation efficiency is determined using Quant-iT RiboGreen RNA assay or different fluorescent based assay.

### C. Agent Cargo

The agent present in the nanoparticle may be a nucleic acid, small molecule drug, protein, polypeptide, antibody, peptide or a combination thereof. The nucleic acid maybe any type of nucleic acid. The nucleic acid may be described as a therapeutic and/or prophylactic nucleic acid. Nucleic acid can be any single stranded DNA or RNA, either double-stranded DNA or the RNA-RNA or DNA-RNA hybrids; shortmers, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof. In certain aspects, the nucleic acid is a plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, viral DNA, circular RNA (circRNA), precursor messenger RNA (pre-mRNA), microRNA (miRNA), guide RNA (gRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, viral satellite RNA, or a combination thereof.

Double-stranded DNA may include, but are not limited to, genes of interest for protein production (antigen protein), genome editing component (such as Cas9 and prime editors), and mobile genetic elements. Double-stranded RNA may include, but are not limited to, small-interfering RNA (siRNA) and other RNA-interference (RNAi) molecules. And single-stranded nucleic acids include, but are not limited to, messenger RNA, antisense oligonucleotides (ASO), and microRNA (miRNA).

Examples of messenger RNA suitable for use in the disclosed lipid nanoparticles include, but are not limited to, Firefly luciferase (Fluc), Nanoluciferase (Nluc), Green Fluorescent Protein (GFP), Cre recombinase, Transposase, Cas9 endonuclease, Cas13 endonuclease, Prime editor, Base editor, Spike protein of SARS-CoV-2, Human soluble angiotensin-converting enzyme 2 (ACE2), Human erythropoietin (EPO), Human alpha-galactosidase, Human Factor IX (FIX), Human Factor XI (FXI), Human cystic fibrosis transmembrane conductance regulator (CFTR), Human epithelial sodium channel (ENaC), Human interleukins (ILs), Human transcription factor EB (TFEB), or a combination thereof.

The nucleic acid optionally may have one or more modifications that confer stability to the nucleic acid (e.g., compared to a wild-type or native version of the nucleic acid), one or more modification that reduce side-effect of nucleic acid, and/or may also comprise one or more modifications relative to the wild-type which correct a defect implicated in the disease-associated, aberrant expression of the protein.

The molar ratio between ionizable lipid and nucleic acid may vary from about 1:1 to about 30:1, such as 1:1, 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. This ratio may include every charged group in a molecule. Similarly, the wt/wt ratio of total lipid component to a therapeutic and/or prophylactic nucleic acid may be from about 2:1 to about 60:1, such as 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1.

### V. In Vivo Transfection

The lipids of the nanoparticle may facilitate transfection, for example, by interacting with endosomal membranes. Although all lipids may be involved in transfection, the ionizable lipid may be the main component responsible for gene delivery due to, e.g., the electrostatic attraction between the negatively charged endosomal membrane and positively charged ionizable lipid in the LNPs. Additionally, the nanoparticle composition may induce cytotoxicity. Transfection efficiency, cytotoxicity, and variability of these parameters as a function of administered dose are valuable metrics to evaluate the feasibility of in vivo studies.

In some aspects, the effectiveness of a particular nanoparticle may be measured by any suitable metric, such as, but not limited to, polypeptide or protein translation (indicated by polypeptide or protein expression). The various amounts of nanoparticles and/or associated nucleic acid introduced may produce various levels of polypeptide or protein expression depending on the dose (the amount of the nanoparticle and/or associated nucleic acid introduced to the cells). Additionally, a nanoparticle composition and/or associated nucleic acid may induce cytotoxicity, or noticeable extent of cell injury and death upon exposure to the nanoparticle and/or associated nucleic acid. In some aspects, a desirable cell viability is at least 50%.

### VI. In Vivo Formulation Studies

To evaluate how effectively various nanoparticle compositions deliver therapeutic and/or prophylactic nucleic acids to target cells, different nanoparticle compositions were prepared and administered to rodents. In certain aspects, mice receive a single dose of LNPs via intravenous, intramuscular, intradermal, or other administration routes. Dose sizes usually range from 0.05 mg/kg to 10 mg/kg or more, where 10 mg/kg describes a dose including 10 mg of a nucleic acid in a nanoparticle for each 1 kg of body mass of the mouse. Upon administration of nanoparticle compositions to mice, dose delivery profiles and dose responses were measured by bioluminescence imaging. For nanoparticle compositions including mRNA, time courses of protein expression can also be assessed. Higher levels of protein expression induced may indicate higher mRNA translation and/or enhanced mRNA delivery efficiency via a given nanoparticle composition. As the non-RNA components are not considered to produce protein expression themselves, a level of protein expression is likely indicative of a delivery efficiency of a nanoparticle composition including nucleic acids.

### VII. Methods of Using the Composition

Nanoparticles comprising one or more of the disclosed compounds may be used to deliver a desired nucleic acid to a subject, such as a human or animal subject. The amount of the nanoparticle administered to the subject can be determined by a person of ordinary skill in the art and may depend on the amount of the nucleic acid to be delivered and the ratio of nucleic acid to lipid, as described herein. The exact amount of a compound required to achieve an effective amount will vary from subject to subject, depending, for example, on species, age, and general condition of a subject, severity of the side effects or disorder, identity of the particular compound, mode of administration, and the like. In some aspects, an effective amount is included in a single dose (e.g., single oral dose) or multiple doses (e.g., multiple oral doses). In certain aspects, when multiple doses are administered to a subject or applied to a tissue or cell, any two doses of the multiple doses include different or substantially the same amounts of a compound described herein. In certain aspects, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks.

Dose sizes may range from 0.05 mg/kg to 10 mg/kg or more, where 10 mg/kg describes a dose including 10 mg of a nucleic acid in a nanoparticle for each 1 kg of body mass of the subject.

Exemplary administration routes include any route suitable to administer the nanoparticle to subject, such as intravenous, intramuscular, intradermal, subcutaneous, intravitreous, subretinal, inhalation or a combination thereof. The nanoparticles are provided in physiological buffers, for example, phosphate buffered saline (PBS), and Hank's balanced salt solution (HBSS), at pH 7.0-7.6.

Lipid nanoparticle formulations may also contain additional pharmaceutical excipients including, but not limited to, diluents, binders, and stabilizers of natural, semisynthetic, and/or synthetic origin. Some examples of these excipients include sugars, such as lactose, sucrose, trehalose, glucose, dextrin; naturally occurring polymers and starches, such as cellulose, chitosan, and derivatives; and synthetic polymers, such as polyethylene glycols, poloxamers, and polyamides.

### VIII. Examples

### General Experimental Considerations:

All reactions were performed using oven-dried glassware equipped with a magnetic stir bar under a atmosphere of nitrogen unless otherwise stated. An oil bath was used for all reactions that require heating. All reagents were purchased from commercial suppliers and used without further purification. ¹H NMR and ¹³C spectra were obtained using a Bruker 400 MHz Avance NEO NanaoBay spectrometer at the Nuclear Magnetic Resonance (NMR) Core Facility. Chemical shifts are reported in ppm and referenced to the CHCl₃ singlet at 7.26 ppm or the center of the ¹³CDCl₃ triplet at 77.16 ppm. Mass spectra were obtained by direct injection into an expression^{L} CMS (Advion Interchim Scientific, US) using an ESI probe.

### Example 1

### Isonitrile Synthesis

Isonitriles (D) were synthesized using a two-step procedure shown in Scheme 4.
Step 1: Amine were converted into their respective formamides by refluxing with formic acid in toluene.
Step 2: The resulted formamide was dehydrated using either (a) POCl₃ or (b) phosegene.

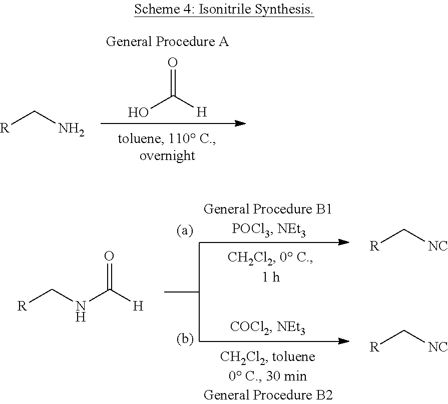

### General Procedure A:

A 500 mL Schlenk Flask was charged with amine (1 equiv.) in anhydrous toluene (0.5 M) under N₂-atmosphere. Under vigorous stirring formic acid (3 equiv.) was added dropwise. The reaction mixture was stirred for 20 minutes at room temperature and then refluxed at 110° C. overnight. After allowing the reaction mixture to cool to room temperature all volatiles were removed under reduced pressure and the leftover was purified by vacuum distillation or column chromatography.

### General Procedure B1:

A 500 mL Schlenk Flask was charged with formamide (1 equiv.) and NEt₃ (5 equiv.) in CH₂Cl₂ (0.8M) under N₂-atmosphere. The mixture was cooled with an ice bath and POCl₃ (1 equiv.) was added dropwise. After stirring the reaction mixture for another 20 minutes the completion of the reaction was indicated by TLC and the reaction mixture was transferred directly onto a silica column and eluted with 0-100% DCM in Et₂O.

### General Procedure B2:

A 500 mL Schlenk Flask was charged with formamide (1 equiv.) and NEt₃ (3.0 M) and CH₂Cl₂ (0.5 M) under N2-atmosphere. The nitrogen line was additionally equipped with a bubbler containing a solution of KOH to quench any exhaust gases. The mixture was cooled with an ice bath and phosgene (1.06 equiv., 15 wt. % in toluene) was added dropwise. The reaction was allowed to warm up and stirred overnight. The precipitate was filtered off and the solution tightened under reduced pressure. The remaining mixture was directly loaded onto a silica column and eluted with 0-100% DCM in Et₂O.

### Example 2

### Synthesis of D1(a¹=1, a²=1, a³=1): 3-Isocyano-N,N-dimethylpropan-1-amine

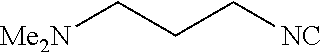

Prepared according to General Procedure A and B1 using N¹,N¹-dimethylpropane-1,3-diamine (24.39 mL, 195.73 mmol). The formamide was purified by vacuum distillation (150° C., 0.3 mTorr) to yield the formamide N-(3-(dimethylamino)propyl)formamide (21.20 g, 195.73 mmol, 83%) as colorless oil. Dehydration using General Procedure B1 yielded the isonitrile D1(a¹=1, a²=1, a³=1) (2.58 g, 23.00 mmol, 17%) as yellow liquid. ¹H NMR (400 MHz, CDCl₃) δ 3.48-3.42 (m, 2H), 2.38 (t, J=6.8 Hz, 2H), 2.20 (s, 6H), 1.85-1.76 (m, 2H). ¹³C NMR (100 MHz, CDCl₃) δ 156.0 (t, J=5.7 Hz), 55.9, 45.5, 39.6 (t, J=6.5 Hz), 27.4.

### Example 3

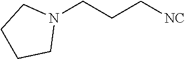

### Synthesis of D2(a=1, b=1): 1-(3-isocyanopropyl)pyrrolidine

Prepared according to General Procedure A and B1 using 3-(pyrrolidine-1-yl)propan-1-amine (15.00 g, 116.99 mmol). The formamide was used without further purification. Dehydration using procedure B1 yielded isonitrile D2(a=1, b=1) (0.47 g, 3.39 mmol, 3%) as colorless liquid. ¹H NMR (400 MHz, CDCl₃) δ 3.50-3.43 (m, 2H), 2.57 (t, J=7.0 Hz, 2H), 2.52-2.46 (m, 4H), 1.92-1.82 (m, 2H), 1.82-1.74 (m, 4H). ¹³C NMR (100 MHz, CDCl₃) δ 156.0 (t, J=5.7 Hz), 54.2, 52.7, 39.8 (t, J=6.5 Hz), 28.60, 23.57.

### Example 4

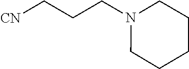

### Synthesis of D2(a=1, b=2): 1-(3-isocyanopropyl)piperidine

Prepared according to General Procedure A and B2 using 3-(piperidin-1-yl)propan-1-amine (25.00 g, 175.75 mmol). The formamide was purified by vacuum distillation (170° C., 0.1 mTorr) to yield N-(3-(piperidin-1-yl)propyl)formamide as colorless oil (28 g, 164.46 mmol, 94%). Dehydration procedure B2 yielded 1-(3-isocyanopropyl)piperidine (7.5 g, 49.26 mmol, 75%) as colorless liquid. ¹H NMR (400 MHz, CDCl₃) δ 3.49-3.41 (m, 1H), 2.47-2.29 (m, 3H), 1.90-1.80 (m, 1H), 1.57 (p, J=5.6 Hz, 2H), 1.48-1.36 (m, 1H). ¹³C NMR (100 MHz, CDCl₃) δ 155.9 (t, J=5.9 Hz), 55.4, 54.7, 39.9 (t, J=6.4 Hz), 26.7, 26.0, 24.4.

### Example 5

### Synthesis of D3(a=0): 4-(2-isocyanoethyl)morpholine

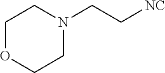

Prepared according to General Procedure A and B1 using 2-morpholinoethan-1-amine (10.00 g, 76.81 mmol). The formamide was purified by vacuum distillation (150° C., 0.3 mTorr) to yield N-(2-morpholinoethyl)formamide (8.4 g, 53.10 mmol, 69%) as colorless oil. Dehydration using procedure B1 yielded isonitrile D3(a=0) (1.30 g, 9.27 mmol, 19%) as yellow liquid. All analytical data were according to published results.

### Example 6

### Synthesis of D3(a=1): 4-(3-isocyanopropyl)morpholine

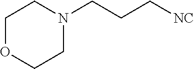

Prepared according to General Procedure A and B1 using 3-morpholinopropan-1-amine (10.00 g, 69.34 mmol). The formamide was purified by vacuum distillation (150° C., 0.3 mTorr) to yield N-(3-morpholinopropyl)formamide (9.8 g, 56.90 mmol, 82%) as colorless oil. Dehydration using procedure B1 yielded isonitrile D3(a=1) (1.7 g, 11.02 mmol, 27%) as yellow liquid. All analytical data were according to published results.

¹H NMR (400 MHz, CDCl₃) δ 3.69 (t, J=4.8 Hz, 4H), 3.52-3.43 (m, 2H), 2.51-2.37 (m, 6H), 1.89-1.77 (m, 2H). ¹³C NMR (100 MHz, CDCl₃) δ 156.2 (t, J=5.7 Hz), 66.93, 54.93, 53.69, 39.6 (t, J=6.5 Hz), 26.13.

### Example 7

### Synthesis of D7(a=1): 3-(2-isocyanoethyl)-1H-indole

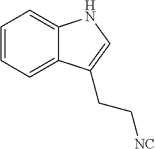

Prepared according to General Procedure A and B1 using tryptamine (14.75 g, 92.06 mmol). The formamide was purified by column chromatography on silica using a gradient of 0-80% EtOAc in hexanes to elute N-(2-(1H-indol-3-yl)ethyl)formamide (13.0 g, 69.06 mmol, 75%) as colorless oil. Dehydration using procedure B1 (at −78° C. instead of 0° C.) yielded isonitrile D7(a=1) (6.90 g, 40.45 mmol, 59%) as white solid. ¹H NMR (400 MHz, CDCl₃) δ 8.13 (s, 1H), 7.56 (dd, J=7.9, 1.1 Hz, 1H), 7.40 (d, J=8.1 Hz, 1H), 7.23 (ddd, J=8.2, 7.0, 1.3 Hz, 1H), 7.19-7.11 (m, 2H), 3.67 (tt, J=7.0, 1.9 Hz, 2H), 3.18 (tt, J=7.1, 2.0 Hz, 2H). ¹³C NMR (100 MHz, CDCl₃) δ 136.4, 126.9, 122.7, 122.5, 119.8, 118.3, 111.6, 111.2, 42.5 (t, J=6.4 Hz), 26.0.

### Example 8

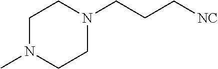

### Synthesis of D6(a=1): 1-(3-isocyanopropyl)-4-methylpiperazine

3-(4-methylpiperazin-1-yl)propan-1-amine was prepared according to General Procedure A using 3-(4-methylpiperazin-1-yl)propan-1-amine (25.00 g, 158 mmol). The formamide was purified by vacuum distillation (170° C., 0.2 mTorr) to yield 3-(4-methylpiperazin-1-yl)propan-1-amine (25.4 g, 137.10 mmol, 86%) as colorless oil. ¹H NMR (400 MHz, CDCl₃) δ 8.10 (s, 1H), 7.09 (s, 1H), 3.45-3.16 (m, 2H), 2.74-2.34 (m, 10H), 2.28 (t, J=2.3 Hz, 3H), 1.79-1.55 (m, 2H).

Dehydration procedure B2 yielded 1-(3-isocyanopropyl)-4-methylpiperazine (0.8 g, 7.17 mmol, 14%) as colorless oil. ¹H NMR (400 MHz, CDCl₃) δ 3.49-3.42 (m, 2H), 2.47 (t, J=6.9 Hz, 9H), 2.30 (s, 2H), 1.88-1.78 (m, 2H). ¹³C NMR (100 MHz, CDCl₃) δ 156.1 (t, J=5.7 Hz), 55.2, 54.5, 53.01, 46.0, 39.7 (t, J=6.5 Hz), 26.6.

### Example 9

### Synthesis of Aldehydes A1, A2, A7

### General Procedure C:

A 1 L Schlenk flask was charged with carboxylic acid (1 equiv.) in THF (0.07 M). At 0° C. lithium aluminum hydride (1.5 equiv.) was added slowly. The solution was allowed to warm up to room temperature and was stirred overnight. The reaction was quenched with sequential additions of water (20 mL), 1N NaOH (20 mL), and water (100 mL) dropwise. The mixture was filtered through celite, and the filtrate was concentrated under reduced pressure and was used without further purification.

### General Procedure D:

The alkyl-alcohol was dissolved CH₂Cl₂ (0.1 M). NaHCO₃ (7 equiv.) was added followed by Dess-Martin periodinane (1.2 equiv.). The mixture was stirred overnight before being diluted in petroleum ether (400 mL). The mixture was then washed sequentially with sat. NaHCO₃ (200 mL) and brine (200 mL), dried over anhydrous sodium sulfate, filtered, and concentrated under reduced pressure. The crude material was purified by column chromatography, and eluted with 0-20% EtOAc in hexanes to afford to corresponding aldehyde.

### Example 10

### Synthesis of A2(x¹=6, y=1, x²=5): Olealdehyde

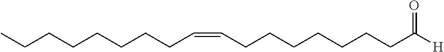 [SMILES: CCCCCCCC/C=C\CCCCCCCC=O]

Prepared according to General Procedure C and D using oleic acid (10.06 mL, 31.86 mmol) to yield olealdehyde as yellow oil (4.90 g, 18.39 mmol, 57%) which was used without further purification.

### Example 11

### Synthesis of A2(x¹=3, y=2, x²=5): (9Z,12Z)-octadeca-9,12-dienal

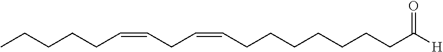 [SMILES: CCCCC/C=C\C/C=C\CCCCCCCC=O]

Prepared according to General Procedure C and D using linoleic acid (10.00 mL, 32.09 mmol) to yield (9Z,12Z)-octadeca-9,12-dienal as yellow oil (5.60 g, 21.18 mmol, 66%), which was used without further purification.

### Example 12

### Synthesis of Branched Aldehydes A5 and Acids C5

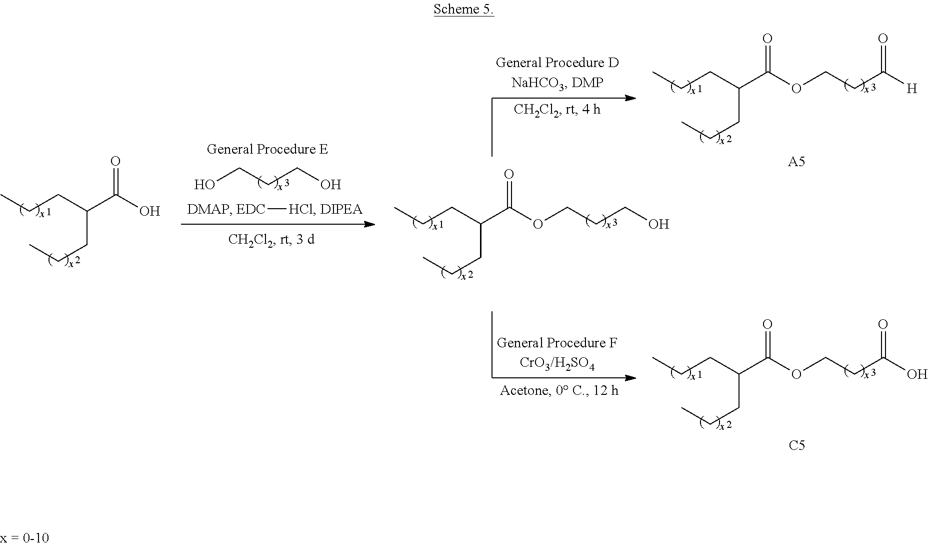

### General Procedure E:

To a solution of carboxylic acid (1 equiv.) in CH₂Cl₂ (0.1 M) were added DIPEA (3 equiv.), EDC (1.5 equiv.) and DMAP (0.5 equiv.). The reaction mixture was stirred for 15 minutes. Diol (3 equiv.) was added and the reaction mixture was stirred for 3 days. The reaction mixture was then diluted with CH₂Cl₂ (200 mL) and washed with water (200 mL), brine (200 mL), and concentrated under reduced pressure. The crude material was purified by column chromatography, eluted with 0-20% EtOAc in hexanes to afford to corresponding alkyl-alcohol.

### General Procedure F:

A 500 mL Schlenk flask was charged with alkyl-alcohol (1 equiv.) in acetone (0.15 M) under N2-atmosphere. The mixture was cooled to 0° C. and Jones reagent (1.2 equiv.) was added dropwise. The mixture was allowed to warm up to room temperature and stirred overnight. The mixture was diluted with CH₂Cl₂ (200 mL), basified to pH 6 and extracted with CH₂Cl₂ (200 mL).

The organic extracts were dried over MgSO₄, filtered and concentrated under reduced pressure to yield the corresponding carboxylic acid, which was purified by column chromatography using 0-20% EtOAc in hexanes as solvent.

### Example 13

### 6-Hydroxyhexyl 2-hexyldecanoate

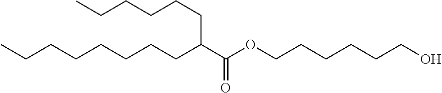 [SMILES: CCCCCCCCC(CCCCCC)C(=O)OCCCCCCO]

Prepared according to General Procedure E using 2-hexyldecanoic acid (11.36 mL, 39.00 mmol) and hexane-1,6-diol (14.40 mL, 116.99 mmol). The crude material was purified by column chromatography, eluted with 0-20% EtOAc in hexanes to afford to corresponding alcohol 6-hydroxyhexyl 2-hexyldecanoate (10.90 g, 30.57 mmol, 78%) as colorless oil. All analytical data are according to published results.

¹H NMR (400 MHz, CDCl₃) δ 4.07 (t, J=6.6 Hz, 2H), 3.64 (t, J=6.6 Hz, 2H), 2.35-2.26 (m, 1H), 1.69-1.51 (m, 6H), 1.48-1.35 (m, 7H), 1.26 (s, 20H), 0.90-0.84 (m, 6H). ¹³C NMR (100 MHz, CDCl₃) δ 176.9, 64.1, 63.0, 46.0, 32.8, 32.7, 32.0, 31.9, 29.7, 29.6, 29.4, 29.4, 28.9, 27.6, 27.6, 25.9, 25.5, 22.8, 22.7, 14.3, 14.2.

### Example 14

### Synthesis of A4(x¹=6, x²=4, x³=5): 6-oxohexyl 2-hexyldecanoate

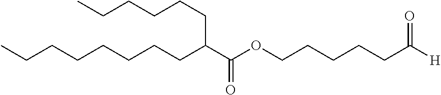 [SMILES: CCCCCCCCC(CCCCCC)C(=O)OCCCCCC=O]

Prepared according to General Procedure D using 6-hydroxyhexyl 2-hexyldecanoate (5.20 g, 14.58 mmol) to yield 6-oxohexyl 2-hexyldecanoate (4.5 g, 12.69 mmol, 87%) as a pale-yellow oil, which was used without further purification.

### Example 15

### Synthesis of C5(x¹=6, x²=4, x³=5): 6-((2-hexyldecanoyl)oxy)hexanoic acid

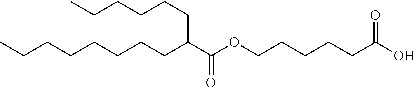 [SMILES: CCCCCCCCC(CCCCCC)C(=O)OCCCCCC(=O)O]

Prepared according to General Procedure F using 6-hydroxyhexyl 2-hexyldecanoate (4.85 g, 13.60 mmol). Column chromatography using 0-20% EtOAc in hexanes as solvent yielded 6-((2-hexyldecanoyl)oxy)hexanoic acid (4.10 g, 11.06 mmol) as colorless oil. ¹H NMR (400 MHz, CDCl₃) δ 10.27 (s, 1H), 4.03 (t, J=6.6 Hz, 2H), 2.34-2.22 (m, 3H), 1.67-1.48 (m, 6H), 1.42-1.32 (m, 4H), 1.22 (s, 20H), 0.83 (t, J=6.8 Hz, 6H). ¹³C NMR (100 MHz, CDCl₃) δ 179.6, 176.8, 63.8, 45.9, 33.9, 32.6, 31.9, 31.8, 29.6, 29.5, 29.3, 29.3, 28.5, 27.5, 27.5, 25.6, 24.3, 22.7, 22.6, 14.1, 14.1.

### Example 16

### Synthesis of 2-(octyldisulfaneyl)pyridine

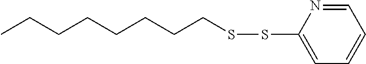 [SMILES: CCCCCCCCSSc1ccccn1]

A 500 mL Schlenk flask was filled with EtOH (210 mL), digydropyridine (25.0 g, 113.47 mmol) under N₂-atmosphere. A drop funnel was equipped onto the Schlenk flask and charged with octane-1-thiol (9.8 mL, 56.74 mmol) in EtOH (100 mL), which was added dropwise to the reaction mixture. The reaction was stirred overnight and monitored by TLC. Upon completion the solvent was removed and the residue was purified by column chromatography on silica using 5% EtOAc in hexanes as solvent to elute 2-(octyldisulfaneyl)pyridine as colorless liquid (12.3 g, 48.15 mmol, 85%).

### Example 17

### Synthesis of 2-(octyldisulfaneyl)ethan-1-ol

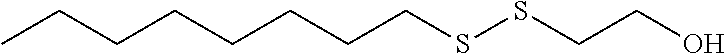

A 500 mL Schlenk flask was filled with EtOH (210 mL), 2-(octyldisulfaneyl)pyridine (12.3 g, 48.15 mmol) and acetic acid (0.2 mL). A solution of mercaptoethanol (3.56 mL, 50.56 mmol) in EtOH (20 mL) was added dropwise over 1 hour and the solution was stirred for 24 hours under N2-atmosphere. Upon completion of the reaction the solvent was removed under reduced pressure and the crude material was purified by column chromatography on silica using 15% EtOH in hexanes as solvent to elute 2-(octyldisulfaneyl)ethan-1-ol (10.5 g, 47.21 mmol, 98%).

### Example 18

### Synthesis of 2-(octyldisulfaneyl)ethyl acrylate

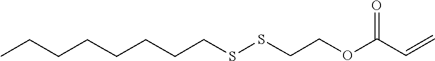 [SMILES: C=CC(=O)OCCSSCCCCCCCC]

A 500 mL Schlenk flask was charged with 2-(octyldisulfaneyl)ethan-1-ol (10.5 g, 47.21 mmol), NEt₃ (9.87 mL, 70.82 mmol) and CH₂Cl₂ (240 mL). Acryloyl chloride (3.84 mL, 47.21 mmol) was added dropwise and stirred overnight. The reaction mixture was washed with 0.1 M HCl (200 mL×3) and dried over MgSO₄. The solvent was removed under reduced pressure to yield an orange oil, which was purified by silica column chromatography using 5% EtOAc in hexanes as solvent to elute 2-(octyldisulfaneyl)ethyl acrylate (10.2 g, 36.90 mmol, 78%) as colorless oil.

### Example 19

### Lipid Synthesis

### General Procedure G: Lipid Synthesis Via Ugi-4C-Reaction

One equivalent of aldehyde, acid and isonitrile was utilized for each NH₂ group contained in the amine (N).

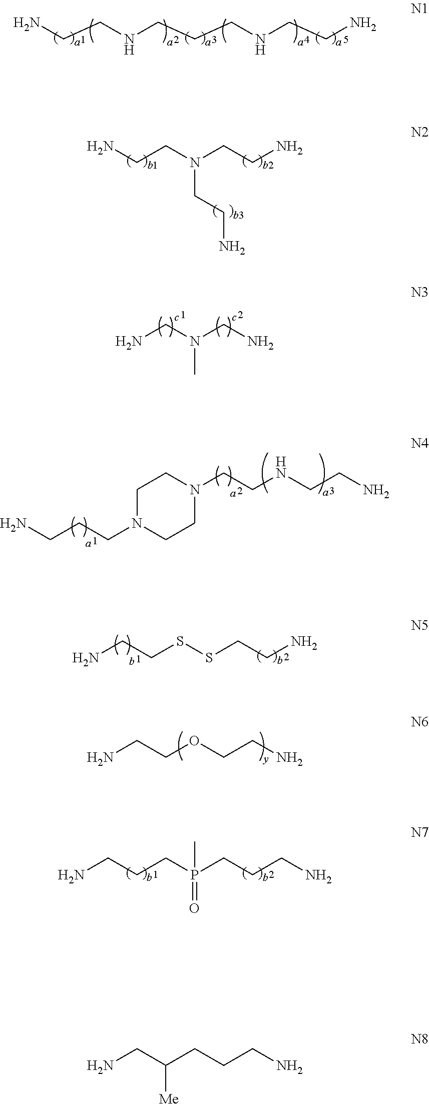

a¹, a², a³, a⁴, a⁵=0-3; b¹, b², b³=0-5; c¹, c²=1-3; y=1-5

Synthesis 1: For amines N1, N3, N4, N5, N6, N7 or N8: A 20 mL scintillation vial was charged with MeOH (100 μL), aldehyde A (0.2 mmol) and amine N (0.1 mmol) and stirred for 3 hours at room temperature using a magnetic stir bar. After subsequent addition of acid C (0.2 mmol) and isonitrile D (0.2 mmol) the reaction mixture was stirred for 24 hours and the solvent was removed under reduced pressure to yield a yellow oil.

Synthesis 2: For amines N2: A 20 mL scintillation vial was charged with MeOH (100 μL), aldehyde A (0.3 mmol) and amine N (0.1 mmol) and stirred for 3 hours at room temperature using a magnetic stir bar. After subsequent addition of acid C (0.3 mmol) and isonitrile D (0.3 mmol) the reaction mixture was stirred for 24 hours and the solvent was removed under reduced pressure to yield a yellow oil.

### General Procedure H: Lipid Synthesis Via Ugi-4C Hybrid Reaction

A 20 mL scintillation vial was charged with MeOH (200 μL), aldehyde A (0.1 mmol) and amine N (0.1 mmol) and stirred for 3 hours at room temperature using a magnetic stir bar. After subsequent addition of acid C (0.1 mmol) and isonitrile D (0.1 mmol) the reaction mixture was stirred overnight. Add 2-(octyldisulfaneyl)ethyl acrylate (0.2 mmol) and stir the reaction mixture overnight at 50° C. Afterwards the solvent was removed under reduced pressure to yield a yellow oil.

### Example 20

Thor 1: Prepared according to General Procedure G to yield a pale-yellow oil. MS (ESI⁺) m/z: [M+H]⁺ Calculated for C₉₄H₁₈₃N₈O₄⁺ 1488.4; Found 1489.9.

### Example 21

Thor 4: Prepared according to General Procedure G to yield a pale-yellow oil. MS (ESI⁺) m/z: [M+H]²⁺ Calculated for C₉₄H₁₈₃N₈O₄⁺ 1037.4 Found 1036.1; [M+H]³⁺ Calculated for C₉₄H₁₈₃N₈O₄⁺ 1037.4 Found 1036.1.

### Example 22

Thor 4-F3: Prepared according to General Procedure G to yield a pale-yellow oil. Fraction was isolated by flash column chromatography on KP—NH (amine modified column from Biotage) using 100% CH₂Cl₂ to 75% CH₂Cl₂, 22% MeOH, 3% NH₄OH. MS (ESI⁺) m/z: [M+H]⁺ Calculated for C₉₀H₁₇₅N₈O₄⁺ 1432.3 Found 1432.1.

### Example 23

Thor 4-F4: Prepared according to General Procedure G to yield a pale-yellow oil. Fraction was isolated by flash column chromatography on KP—NH (amine modified column from Biotage) using 100% CH₂Cl₂ to 75% CH₂Cl₂, 22% MeOH, 3% NH₄OH. MS (ESI⁺) m/z: [M+H]⁺ Calculated for C₉₆H₁₈₉N₁₀O₄⁺ 1546.4 Found 1546.1.

### Example 24

Thor-6: Prepared according to General Procedure G to yield a pale-yellow oil. MS (ESI⁺) m/z: [M+H]⁺ Calculated for C₉₄H₁₇₅N₈O₄⁺ 1480.3 Found 1481.1.

### Example 25

Thor-7: Prepared according to General Procedure G to yield a pale-yellow oil. MS (ESI⁺) m/z: [M+H]⁺ Calculated for C₉₁H₁₆₈N₇O₄⁺ 1423.3 Found 1424.1.

### Example 26

Thor-8: Prepared according to General Procedure G to yield a pale-yellow oil. MS (ESI⁺) m/z: [M+H]⁺ Calculated for C₉₀H₁₆₅N₆O₄⁺ 1395.3 Found 1395.2.

### Example 27

Thor-9: Prepared according to General Procedure G to yield a pale-yellow oil. MS (ESI⁺) m/z: [M+H]²⁺ Calculated for C₁₃₂H₂₄₁N₁₀O₆⁺ 1031.4 Found 1032.4.

### Example 28

Thor-10: Prepared according to General Procedure G to yield a pale-yellow oil. MS (ESI⁺) m/z: [M+H]²⁺ Calculated for C₁₃₅H₂₄₇N₁₀O₆⁺ 1052.5 Found 1053.6.

### Example 29

Thor-11: Prepared according to General Procedure G to yield a pale-yellow oil. MS (ESI⁺) m/z: [M+H]⁺ Calculated for C₁₀₈H_{2O7}N₈O₈⁺ 1744.6 Found 1745.6.

### Example 30

Thor-12: Prepared according to General Procedure G to yield a pale-yellow oil. MS (ESI⁺) m/z: [M+H]²⁺ Calculated for C₁₀₅H₂₀₀N₇O₈⁺ 843.8 Found 844.7.

### Example 31

Thor-13: Prepared according to General Procedure G to yield a pale-yellow oil. MS (ESI⁺) m/z: [M+H]⁺ Calculated for C₁₀₄H₁₉₇N₆O₈⁺ 1658.5 Found 1659.5.

### Example 32

Thor-14: Prepared according to General Procedure G to yield a pale-yellow oil. MS (ESI⁺) m/z: [M+H]²⁺ Calculated for C₁₅₃H₂₈₉N₁₀O₁₂⁺ 1229.6 Found 1230.5.

### Example 33

Thor-15: Prepared according to General Procedure G to yield a pale-yellow oil. MS (ESI⁺) m/z: [M+H]²⁺ Calculated for C₁₅₆H₂₉₅N₁₀O₁₂⁺ 1250.6 Found 1252.1.

### Example 34

Thor-16: Prepared according to General Procedure G to yield a pale-yellow oil. MS (ESI⁺) m/z: [M+H]²⁺ Calculated for C₁₀₂H₁₉₉N₈O₈⁺ 1664.5 Found 920.5.

### Example 35

Thor-17: Prepared according to General Procedure G to yield a pale-yellow oil. MS (ESI⁺) m/z: [M+H]⁺ Calculated for C₉₉H₁₉₂N₇O₈⁺ 1607.5 Found 1150.9.

### Example 36

Thor-18: Prepared according to General Procedure G to yield a pale-yellow oil. MS (ESI⁺) m/z: [M+H]⁺ Calculated for C₁₄₄H₂₇₇N₁₀O_{12+2339.1} Found 873.6.

### Example 37

Thor-19: Prepared according to General Procedure G to yield a pale-yellow oil. MS (ESI⁺) m/z: [M+H]⁺ Calculated for C₁₄₇H₂₈₃N₁₀O₁₂⁺ 2381.1 Found 776.7.

### Example 38

Thor-20: Prepared according to General Procedure G to yield a pale-yellow oil. MS (ESI⁺) m/z: [M+H]²⁺ Calculated for C₁₁₀H₂₁₅N₈O₁₂⁺ 920.3 Found 920.5.

### Example 39

Thor-21: Prepared according to General Procedure G to yield a pale-yellow oil. MS (ESI⁺) m/z: [M+H]²⁺ Calculated for C₁₀₇H₂₀₈N₇O₁₂⁺ 891.8 Found 892.4.

### Example 40

Thor-22: Prepared according to General Procedure G to yield a pale-yellow oil. MS (ESI⁺) m/z: [M+H]²⁺ Calculated for C₁₅₆H₃₀₁N₁₀O₁₈⁺ 2603.29. Found 776.6.

### Example 41

Thor-23: Prepared according to General Procedure G to yield a pale-yellow oil. MS (ESI⁺) m/z: [M+H]²⁺ Calculated for C₁₅₉H₃₀₇N₁₀O₁₈⁺ 2645.3 Found 776.6.

### Example 42

Thor-24: Prepared according to General Procedure G to yield a pale-yellow oil. MS (ESI⁺) m/z: [M+H]²⁺ Calculated for C₁₅₉H₃₀₇N₁₀O₁₈⁺ 1169.6 Found 1170.2; [M+H]³⁺ Calculated for C₁₅₉H₃₀₇N₁₀O₁₈⁺ 779.7 Found 780.9.

### Example 43

Thor-25: Prepared according to General Procedure G to yield a pale-yellow oil. MS (ESI⁺) m/z: [M+H]²⁺ Calculated for C₁₄₇H₂₈₃N₁₀O₁₂⁺ 1190.6 Found 1191.5. [M+H]³⁺ Calculated for C₁₄₇H₂₈₃N₁₀O₁₂⁺ 793.7 Found 794.9.

### Example 44

Thor-26: Prepared according to General Procedure G to yield a pale-yellow oil. MS (ESI⁺) m/z: [M+H]⁺ Calculated for C₉₀H₁₇₄N₇O₄⁺ 1417.4 Found 1417.0; [M+H]²⁺ Calculated for C₉₀H₁₇₄N₇O₄⁺ 708.7 Found 709.5.

### Example 45

Thor-27: Prepared according to General Procedure G to yield a pale-yellow oil. MS (ESI⁺) m/z: [M+H]⁺ Calculated for C₉₂H₁₇₉N₈O₄⁺ 1460.4 Found 1459.9; [M+H]²⁺ Calculated for C₉₂H₁₇₉N₈O₄⁺ 730.2 Found 731.2.

### Example 46

Thor-28: Prepared according to General Procedure G to yield a pale-yellow oil. MS (ESI⁺) m/z: [M+H]⁺ Calculated for C₉₄H₁₈₁N₈O₄⁺ 1486.4 Found 1487.1; [M+H]²⁺ Calculated for C₉₄H₁₈₁N₈O₄⁺ 743.2 Found 743.6.

### Example 47

Thor-29: Prepared according to General Procedure G to yield a pale-yellow oil. MS (ESI⁺) m/z: [M+H]⁺ Calculated for C₉₄H₁₈₂N₉O₄⁺ 1501.4 Found 745.5.

### Example 48

Thor-30: Prepared according to General Procedure G to yield a pale-yellow oil. MS (ESI⁺) m/z: [M+H]⁺ Calculated for C₁₀₂H₁₉₉N₈O₈⁺ 1664.5 Found 1662.7; [M+H]²⁺ Calculated for C₁₀₂H₁₉₉N₈O₈⁺ 832.3 Found 832.4; [M+H]³⁺ Calculated for C₁₀₂H₁₉₉N₈O₈⁺ 554.8 Found 555.2.

### Example 49

Thor-31: Prepared according to General Procedure G to yield a pale-yellow oil. MS (ESI⁺) m/z: [M+H]⁺ Calculated for C₁₀₂H₁₉₇N₈O₈⁺ 1662.5 Found 1664.6; [M+H]²⁺ Calculated for C₁₀₂H₁₉₇N₈O₈⁺ 831.3 Found 833.5; [M+H]³⁺ Calculated for C₁₀₂H₁₉₇N₈O₈⁺ 554.2 Found 555.8.

### Example 50

Thor-32: Prepared according to General Procedure G to yield a pale-yellow oil. MS (ESI⁺) m/z: [M+H]⁺ Calculated for C₁₀₂H₁₉₈N₉O₈⁺ 1677.5 Found 1678.7; [M+H]²⁺ Calculated for C₁₀₂H₁₉₈N₉O₈⁺ 838.8 Found 839.4.

### Example 51

Thor-33: Prepared according to General Procedure G to yield a pale-yellow oil. MS (ESI⁺) m/z: [M+H]⁺ Calculated for C₉₈H₁₉₁N₈O₁₂⁺ 1672.5 Found 1548.5.

### Example 52

Thor-34: Prepared according to General Procedure G to yield a pale-yellow oil. MS (ESI⁺) m/z: [M+H]⁺ Calculated for C₆₉H₁₈₅N₆O₁₂⁺ 1614.4 Found 1826.9.

### Example 53

Thor-35: Prepared according to General Procedure G to yield a pale-yellow oil. MS (ESI⁺) m/z: [M+H]⁺ Calculated for C₁₃₈H₂₆₅N₁₀O₁₈⁺ 2351.0 Found 1193.

### Example 54

Thor-36: Prepared according to General Procedure G to yield a pale-yellow oil. MS (ESI⁺) m/z: [M+H]⁺ Calculated for C₁₄₁H₂₇₁N₁₀O₁₈⁺ 2393.1 Found 1549.5.

### Example 55

Thor-37: Prepared according to General Procedure H to yield a pale-yellow oil. MS (ESI⁺) m/z: [M+H]⁺ Calculated for C₈₆H₁₆₈N₆O₁₀S₄⁺ 1574.2 Found 1575.1.

### Example 56

Thor-38: Prepared according to General Procedure H to yield a pale-yellow oil. MS (ESI⁺) m/z: [M+H]⁺ Calculated for C₈₃H₁₆₂N₅O₁₀S₄⁺ 1517.1 Found 1517.1.

### Example 57

Thor-39: Prepared according to General Procedure H to yield a pale-yellow oil. MS (ESI⁺) m/z: [M+H]²⁺ Calculated for C₁₃₂H₂₅₅N₈O₁₆S₄⁺ 1168.4 Found 1169.3.

### Example 58

Thor-40: Prepared according to General Procedure H to yield a pale-yellow oil. MS (ESI⁺) m/z: [M+H]²⁺ Calculated for C₈₆H₁₆₈N₆O₁₀S₄⁺ 1189.4 Found 1190.3.

### Example 59

### LNP Formulation and Characterization

LNP, mRNA Materials and Mouse Models

Firefly luciferase CleanCap FLuc mRNA(5moU) was purchased from Trilink BioTechnologies. 1,2-dioleoyl-sn-glycero-3-phosphoethanolamine (DOPE) and 1,2-dimyristoyl-sn-glycero-3-phosphoethanolamine-N-[methoxy(polyethylene glycol)-2000]ammonium salt (C14-PEG) were purchased from Avanti. Cholesterol was obtained from Sigma-Aldrich.

### Lipid Nanoparticles Formulation

Lipid Nanoparticles were synthesized by mixing an ethanolic lipid solution with an aqueous solution of nucleic acids. Nanoparticles were obtained by any suitable mixing process such as, but not limited to, pipet mixing, syringe mixing, T-junction mixing, or microfluidic mixing of two or more solutions and/or suspensions, one of which contains one or more nucleic acids and the other has lipid components. The nucleic acid solution includes an aqueous buffer, such as a 5-100 mM citrate or acetate buffer to maintain the solution at acidic pH, such as at a pH of from less than 7, for example, 2, 3, 4, 5, or 6. Lipid components were dissolved in ethanol. Total flow rate, flow rate ratio was optimized depending on target nanoparticle size, polydispersity, encapsulation efficiency, and other features. A volumetric ratio between nucleic acid solution and lipid solution was varied from about 1:1 to 20:1, such as 3:1. 5:1, 10:1, 15:1, 20:1 or as necessary. The total flow rate may be from about 5 ml/min to 50 ml/min.

For the purification purpose, LNPs were either directly buffer exchanged by using physiological buffer at a 1:1 to 1:10 v/v dilution ratio or dialyzed two times at 4° C. or room temperature, if needed, using phosphate-buffered saline and concentrated with 10-100 kDa centrifuge filter tubes.

Lipid solution contains different lipid components including ionizable lipids, phospholipids, structural lipids and polymer conjugated lipids. The amount of ionizable lipid used in the LNP formulation ranges from 20 to 100 mol % of the total amount of lipids in the nanoparticle or 20 to 100% w/w ratio of nucleic acid to be encapsulated.

The amount of phospholipid used from 0 to 30 mol % of the total lipids. The phospholipid may be any phospholipid suitable to form the nanoparticles, such as, but not limited to, phospholipids disclosed herein.

The amount of structural lipid used from 0 to 70 mol % of the total amount of lipids. The structural lipid used is any suitable structural lipid, such as, but not limited to, structural lipids disclosed herein.

The amount of polymer-conjugated lipid (PEG-lipid) used from 0 to 10 mol % of the total amount of lipids. The PEG lipid is any suitable PEG lipid, such as, but not limited to, polymer-conjugated lipids disclosed herein.

### Characterization of Novel LNP Systems

Size distribution and polydispersity index (PDI) of LNPs were determined via dynamic light scattering using a Stunner (Unchained Labs, US), Zetasizer Nano ZSP (Malvern Instruments, UK) or nanoparticle tracking analyzer (NTA). Nucleic acid encapsulation efficiency was determined using Quant-iT RiboGreen RNA assay or different fluorescent based assay.

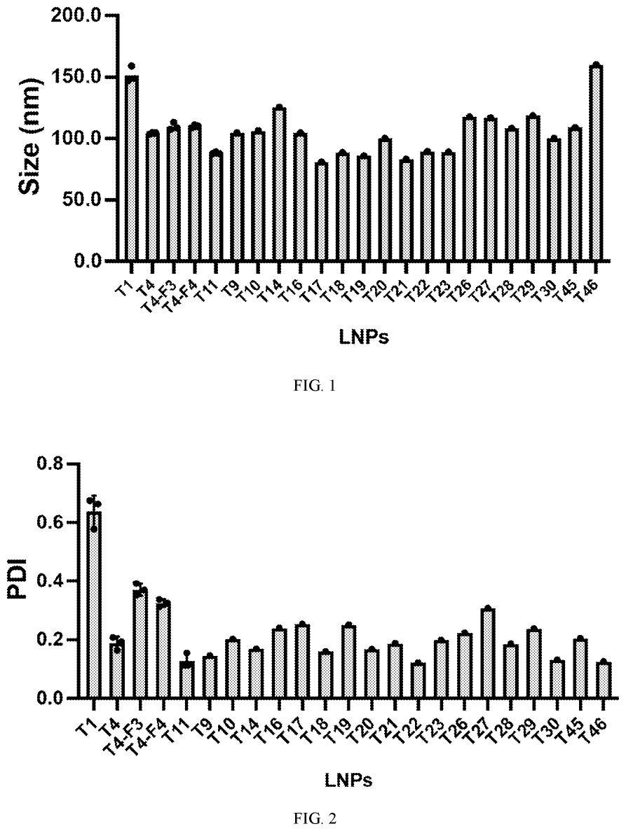

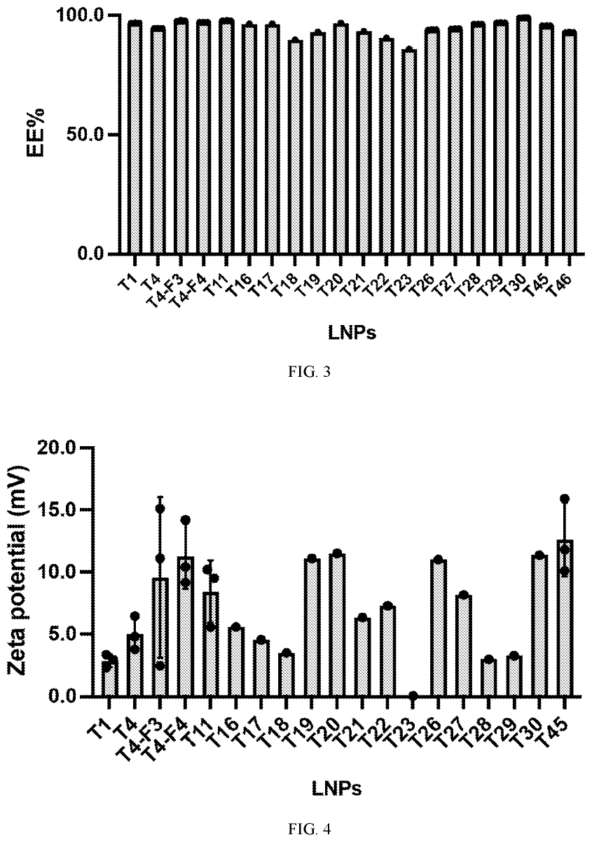

The LNP systems that were studied exhibited a wide range of sizes, with an average diameter ranging from 80.88 nm to 159.96 nm (FIG. 1). All formulations were under the size range of 200 nm, a size range most relevant to physical and biochemical targeting through intravenous injections. Nanoparticles larger than 200 nm typically activate the complement system and are rapidly eliminated from systemic circulation. The Polydispersity Index (PDI) of these lipids ranged from 0.13±0.02 to 0.63±0.05 (FIG. 2). This large range is lipid dependent. Certain lipids disclosed herein demonstrated PDI values of around 0.2, which can be very useful in vivo. All LNPs were observed to have high encapsulation efficiency of mRNA higher than 90% except for T23 which had 85% (FIG. 3). Zeta potential values are used as a measure to assess the surface charge of LNPs to determine if they are anionic, cationic, or neutral. All the formulated LNPs were positively charged with the zeta potential values under +15 mV with the exception of T23 which had a neutral surface charge (FIG. 4).

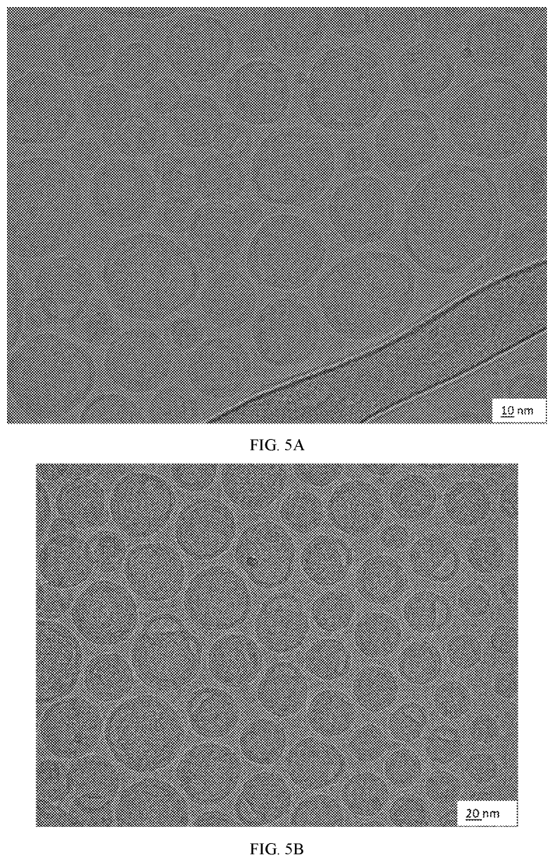

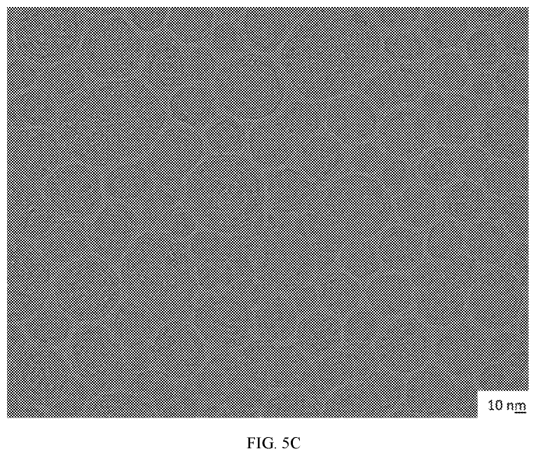

The shape and size of the LNP systems were confirmed through cryo-electron microscopy (cryo-EM). Most of the LNPs displayed a smooth, spherical to oblong shape (FIGS. 5A-5C). However, some LNP systems exhibited heterogeneous size distributions with protrusions resembling blebs.

### Example 60

### In Vivo Experiments

Balbc/J female mice were purchased from the Jackson Laboratory (Bar Harbor, ME, USA). In all experiments, N=2-3 mice/group were used. Mice were injected with 0.1 mg/kg dose of mRNA loaded LNPs via intravenous route for lipid screening. For Cre mRNA delivery, Ai9 mice of either sex were used which were purchased from Jackson Laboratories and bred in the animal facility. These mice were injected with 0.2 mg/kg dose of Cre mRNA loaded LNPs and organs were harvest 7 days after treatment.

### In Vivo Performance of Novel LNP Systems

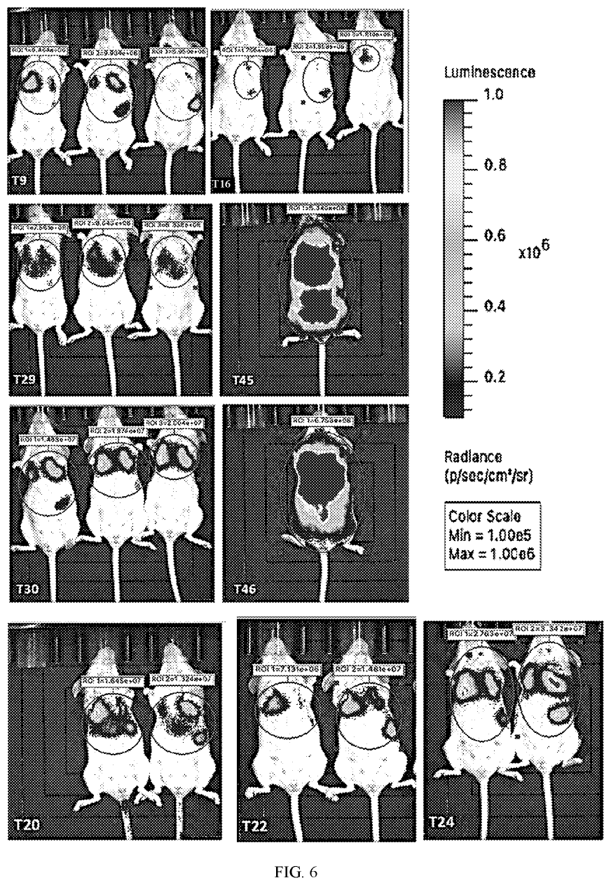

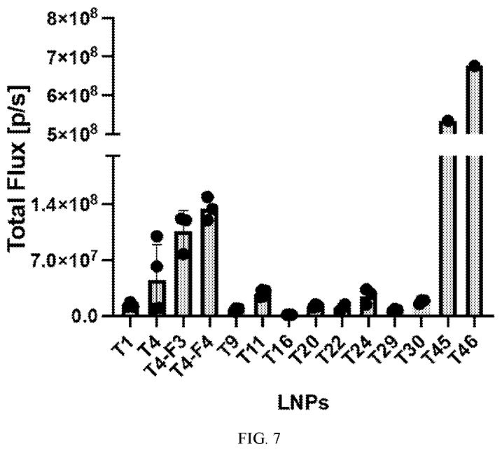

The in vivo transfection efficiency of LNPs encapsulating fluc mRNA was evaluated by administering a total dose of 0.1 mg/kg mRNA per mouse. Following intravenous injection of LNPs, the majority of the particles elicited a robust bioluminescent signal in the lungs of Balb/c mice after 5 hours (FIG. 6). And quantification of bioluminescent signals from lung area was done using IVIS images (FIG. 7). The region of interest was kept constant in all images (n=1-3*). T45 and T46 showed the highest luciferase expression, respectively, compared to other lipids, as observed through live imaging. However, recovery of mRNA was lower and only one mouse per group was injected.

### Ex Vivo Performance of Novel LNP Systems

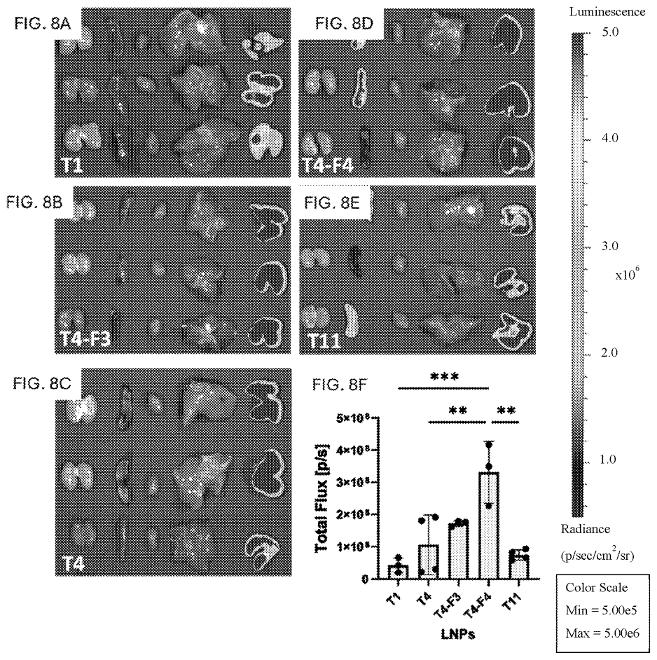

During ex vivo imaging, the luciferase expression was detected exclusively in the lungs of the treated groups. Additionally, in some cases, a signal was also observed in the spleen (FIGS. 8A-8F). This suggested that the LNPs, when administered intravenously, primarily targeted the lungs for luciferase expression, but there was also some distribution or accumulation in the spleen in certain treated groups.

### Example 61

Cre mRNA Delivery to the Ai9 Mouse

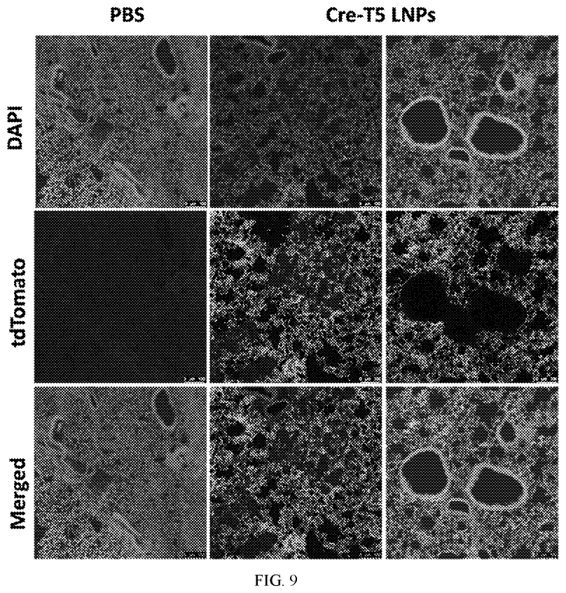

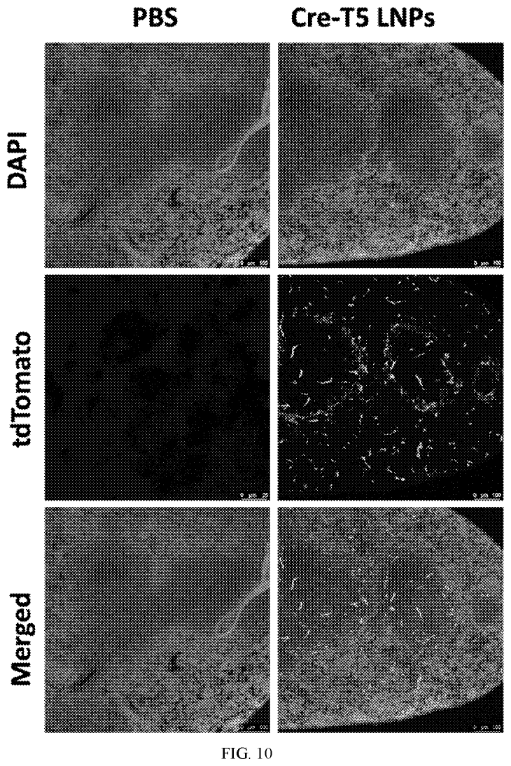

Breeder Ai9 mice were purchased from the Jackson Laboratory. Ai9 is a Cre reporter tool designed to have a loxP-flanked STOP cassette preventing transcription of tdTomato under the control of a ubiquitous promoter. Following Cre-mediated recombination, Ai9 mice express robust tdTomato. Thor-5 lipid was used to deliver the Cre mRNA (Cre-T5 LNPs) into both the lungs and spleen at different molar percentage of lipids. Cre-T5 LNPs showed narrow size distribution, with a hydrodynamic diameter of <100 nm, a PDI value of <0.20 and encapsulation efficiency >91%. The zeta potential of an unmodified LNP was 11.5±1.5 mV. Via systemic injection, 2 μg cre mRNA per mouse was delivered, and organs were harvested after a 10-day injection. Both lungs and spleen were cryo-frozen and sectioned, DAPI stained, and imaged under confocal microscopy. Both the lungs and spleen (FIGS. 9 and 10) showed robust tdTomato expression in different cell types. This robust tdTomato expression can also open the way for gene editing tools in both lung and spleen-related genetic diseases.

In view of the many possible aspects to which the principles of the disclosure may be applied, it should be recognized that the illustrated aspects are only preferred examples of the disclosure and should not be taken as limiting the scope of the disclosure. Rather, the scope of the disclosure is defined by the following claims. We therefore claim as the disclosure all that comes within the scope and spirit of these claims.

We claim:

1. A compound according to Formula I 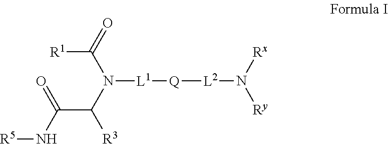
wherein:
L¹ is —(CH₂)ₙ₁—;
L² is —(CH₂)ₙ₂—;
each n1 and n2 independently is from 1, 2, 3, 4, 5, or 6;
Q is selected from —(CR^{a}₂)—, —NH—[(CH₂)ₐNH]_{b}—, —NR^{b}—, —(OCH₂CH₂)ₚO—, —P(═O)(CH₃)—, —S—S—, or 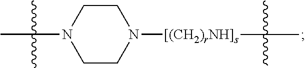
each R^{a} independently is H or C₁₋₆alkyl;
R^{x} is —C(═O)R² or —CH₂CH₂C(═O)OCH₂CH₂S—S—(CH₂)ₜCH₃;
R^{y} is 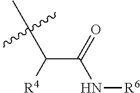
or —CH₂CH₂C(═O)OCH₂CH₂S—S—(CH₂)ₜCH₃;
a is from 2 to 5;
b is from 1 to 3;
r is from 1 to 4;
s is from 0 to 3;
p is from 1 to 5;
each t independently is from 3 to 20;
R^{b} is selected from H, C₁₋₆alkyl, or 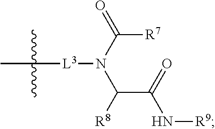
L³ is —(CH₂)ₙ₃—;
n3 is from 1, 2, 3, 4, 5, or 6;
each of R¹, R² and R⁷ if present, independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂;
each of R³, R⁴ and R⁸, if present, independently is selected from —C₅₋₂₅alkyl, —C₅₋₂₅alkenyl, —C₅₋₂₅alkynyl, —(CH₂)ₓ—OC(═O)—(C₅₋₂₅alkyl), —(CH₂)ₓ—C(═O)O—(C₅₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂;
each of R⁵, R⁶, and R⁹, if present, independently is selected from —C₄₋₂₂alkyl, —(CH₂)_{z+2}N(C₁₋₄alkyl)₂, —(CH₂)_{z+2}(5- or 6-membered heterocyclyl optionally substituted with C₁₋₄alkyl), —C₁₋₆OPG, —(CH₂)_{z+2}(5- to 9-membered heteroaryl), or —(CH₂)_{z+2}N((CH₂)_{z+1}OPG)₂, where PG is a silyl protecting group;
x is from 0 to 10;
y is from 1 to 5; and
z is from 0 to 3.

2. The compound of claim 1, having a Formula II

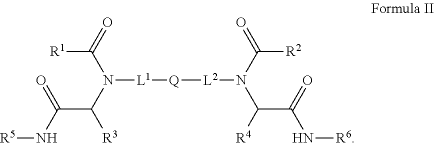

3. The compound of claim 1, having a Formula III

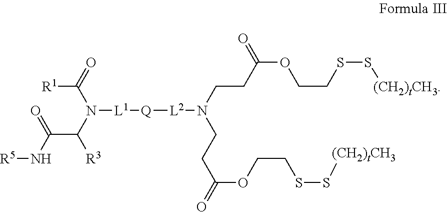

4. The compound of claim 1, wherein:
Q is —(CR^{a}₂)—, and one R^{a} is H and the other R^{a} is C₁₋₆alkyl; or
Q is —NH[(CH₂)ₐNH]_{b}—, a is 2, 3, or 4, and b is 1; or
Q is —NR^{b}—, and R^{b} is C₁₋₆alkyl or 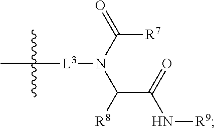
or
Q is 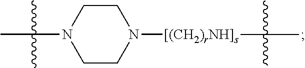
or
Q is —(OCH₂CH₂)ₚO— and p is 1, 2, or 3.

5. The compound of claim 4, wherein Q is 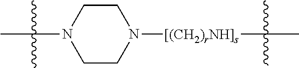
and s is 1 and r is 2.

6. The compound of claim 1, wherein -L¹-Q-L²- is selected from: —(CH₂)₃NH(CH₂)₄NH(CH₂)₃—, —(CH₂)₂NH(CH₂)₄NH(CH₂)₂—, —(CH₂)₃NH(CH₂)₂NH(CH₂)₃—, —(CH₂)₂NH(CH₂)₂NH(CH₂)₂—, —(CH₂)₄N(CH₃)(CH₂)₄—, —(CH₂)₃N(CH₃)(CH₂)₃—, —CH₂C(CH₃)(CH₂)₃—, 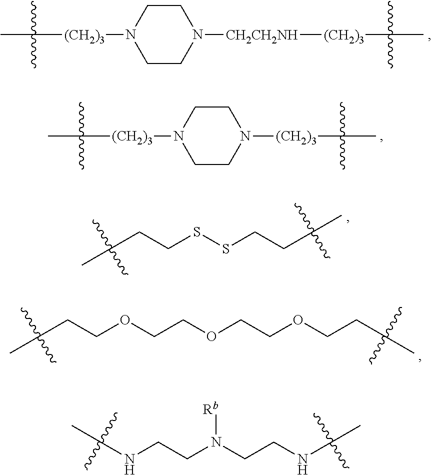
where R^{b} is 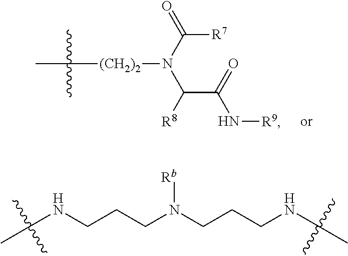
where R^{b} is

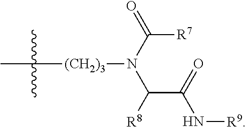

7. The compound of claim 1, wherein:
i) each of R¹, R², and R⁷ if present, independently is selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, where x is from 0 to 6;
ii) each of R³, R⁴, and R⁸ if present, independently is selected from —C₁₀₋₂₀alkyl, —C₁₀₋₂₀alkenyl, —C₁₀₋₂₀alkynyl, —(CH₂)ₓ—OC(═O)—(C₁₀₋₂₅alkyl), —(CH₂)ₓ—C(═O)O—(C₁₀₋₂₅alkyl), —(CH₂)ₓ—(CH₂CH₂O)_{y}—(C₁₋₁₂alkyl), —(CH₂)ₓ₊₂—S—S—(C₁₋₁₂alkyl), or —(CH₂)ₓ₊₁—CH(OC₁₋₁₂alkyl)₂, where x is from 0 to 6; or
iii) a combination of i and ii.

8. The compound of claim 1, wherein:
i) each of R¹ and R², and R⁷ if present, independently is selected from —C₁₀₋₂₀alkenyl, —(CH₂)ₓ—OC(═O)—(C₁₀₋₂₅alkyl), —(CH₂)ₓ—C(═O)O—(C₁₀₋₂₅alkyl), or —(CH₂)ₓ₊₂—S—S—(C₄₋₁₂alkyl), where x is from 0 to 6;
ii) each of R³ and R⁴, and R⁸ if present, independently is selected from —C₁₀₋₂₀alkenyl, —(CH₂)ₓ—OC(═O)—(C₁₀₋₂₅alkyl), or —(CH₂)ₓ₊₂—S—S—(C₁₋₁₂alkyl), where x is from 0 to 6; or
iii) a combination of i and ii.

9. The compound of claim 1, wherein:
i) each of R¹ and R², and R⁷ if present, independently is selected from 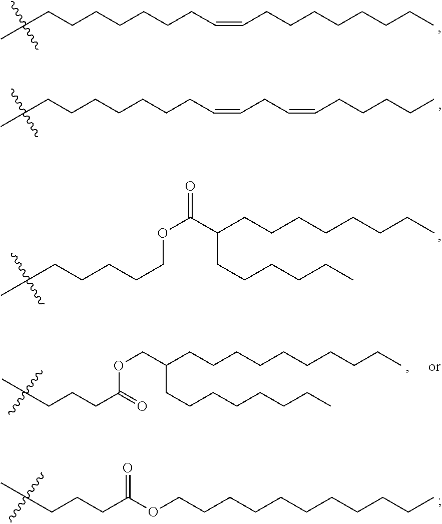 [SMILES: *CCCCCCC/C=C\CCCCCCCC ; *CCCCCCC/C=C\C/C=C\CCCCC ; *CCCCCOC(=O)C(CCCCCC)CCCCCCCC ; *CCCC(=O)OCC(CCCCCCCC)CCCCCCCCCC ; *CCCC(=O)OCCCCCCCCCCC]
ii) each of R³ and R⁴, and R⁸ if present, independently is selected from 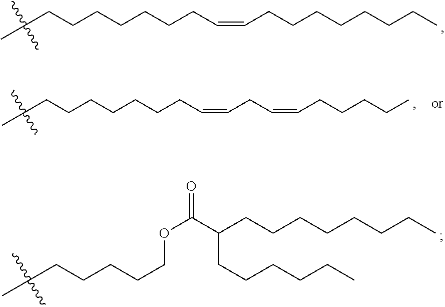 [SMILES: *CCCCCCC/C=C\CCCCCCCC ; *CCCCCCC/C=C\C/C=C\CCCCC ; *CCCCCOC(=O)C(CCCCCC)CCCCCCCC]
iii) each of R⁵, R⁶, and R⁹ if present, independently is selected from (CH₃)₂NCH₂CH₂CH₂—, 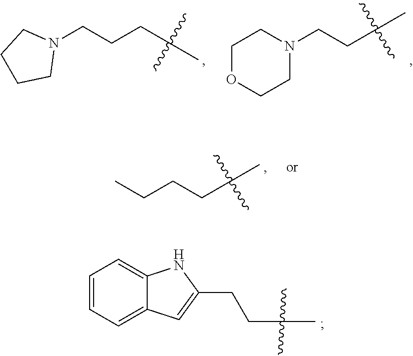
or
iv) a combination of i, ii, or iii.

10. The compound of claim 1, wherein the compound has a structure according to Formula IV, IV-A or IV-B:

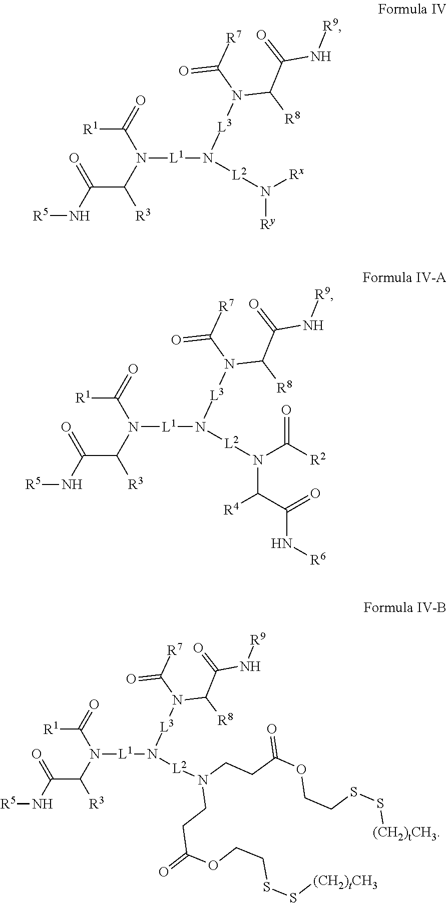

11. The compound of claim 10, wherein at least one of L¹, L² and L³ is different from the other two.

12. The compound of claim 1, wherein:
each of R¹, R² and R⁷, if present, are the same;
each of R³, R⁴ and R⁸, if present, are the same;
each of R⁵, R⁶ and R⁹, if present, are the same;
or any combination thereof.

13. The compound of claim 1, wherein the compound has a structure according to a formula selected from:

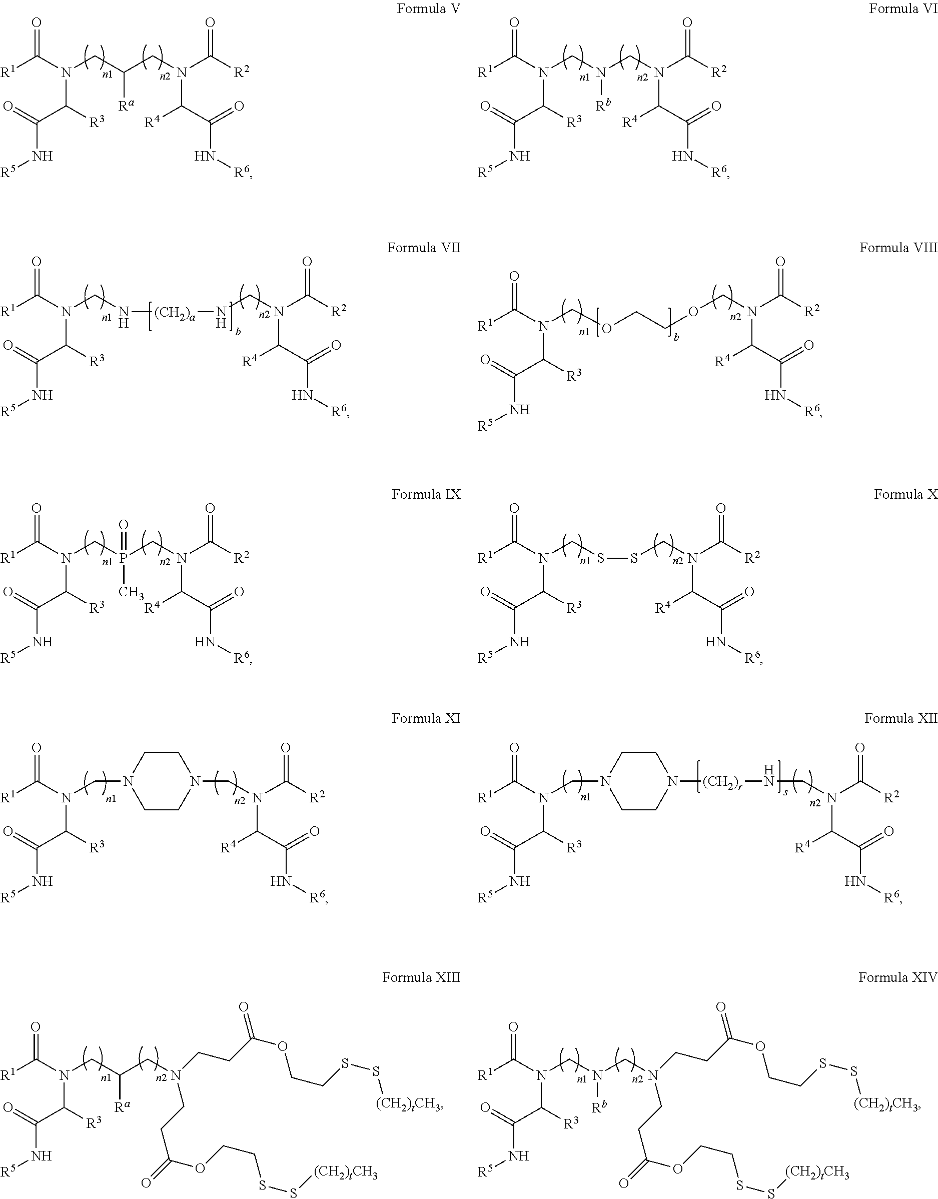

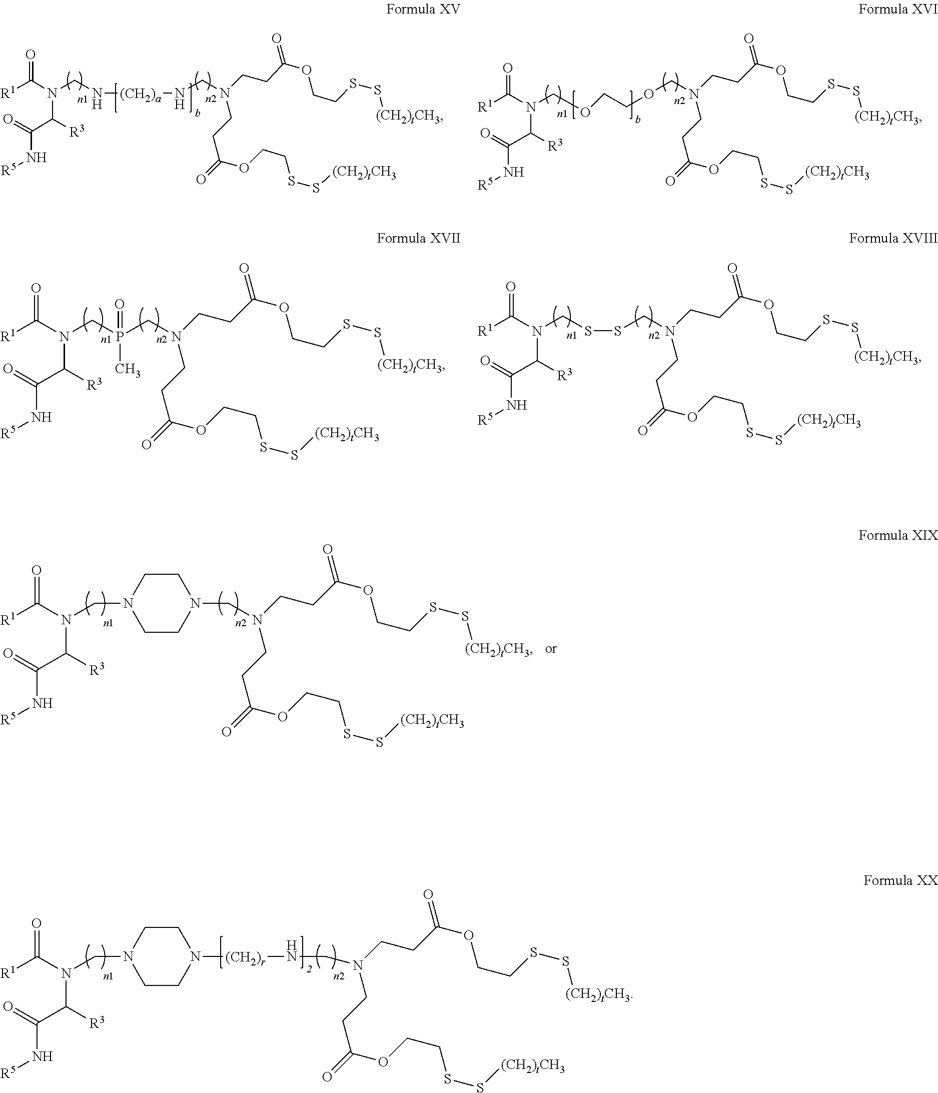

14. The compound of claim 13, wherein:
R^{a} is C₁₋₃alkyl;
R^{b} is C₁₋₃alkyl;
a is 2, 3, or 4;
b is 1, 2, or 3;
r is 1, 2, or 3;
s is 1, 2 or 3; and/or
t is from 3 to 15.

15. The compound of claim 1, wherein each of n1 and n2, and n3 if present, is 2 or 3.

16. The compound of claim 1, wherein the compound is selected from:

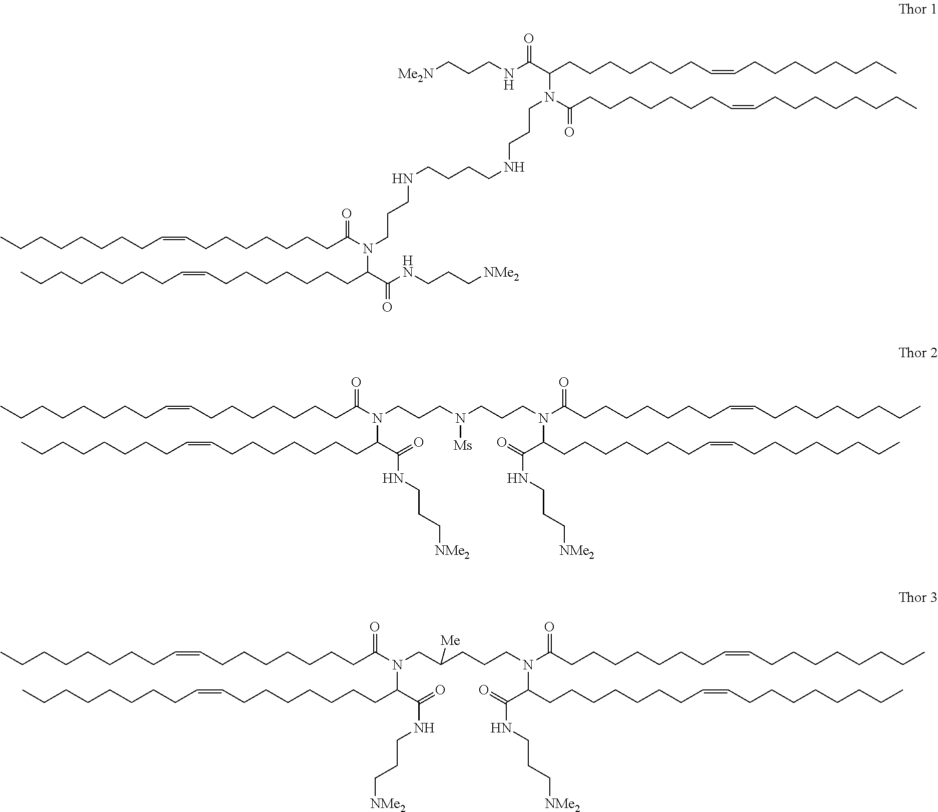

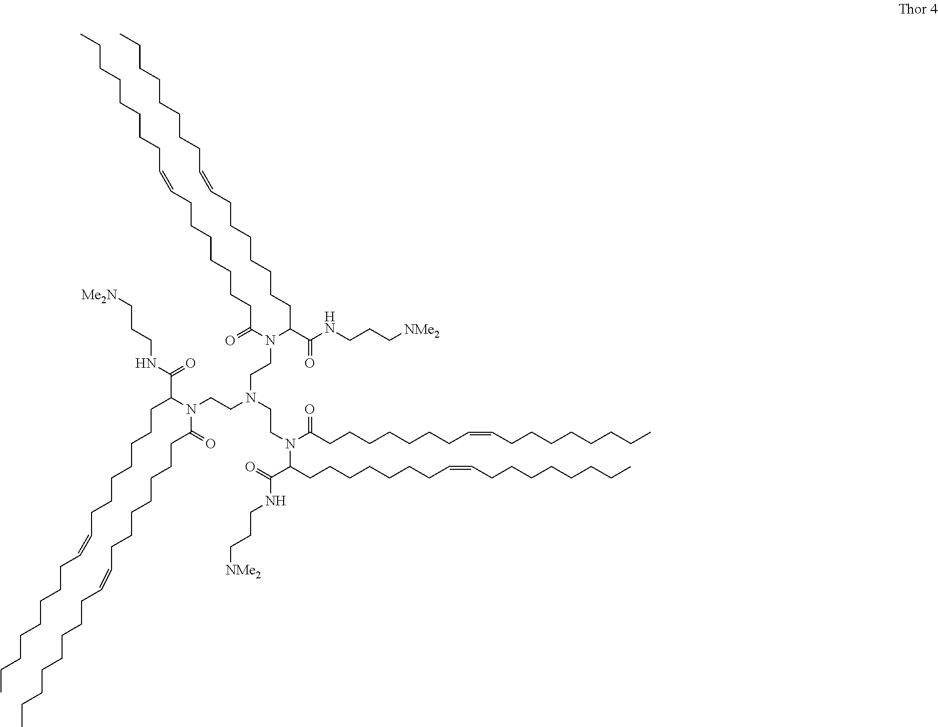 [SMILES: CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCCN(C)C)CCN(C(=O)CCCCCCC/C=C\CCCCCCCC)C(CCCCCCC/C=C\CCCCCCCC)C(=O)NCCCN(C)C)C(CCCCCCC/C=C\CCCCCCCC)C(=O)NCCCN(C)C]

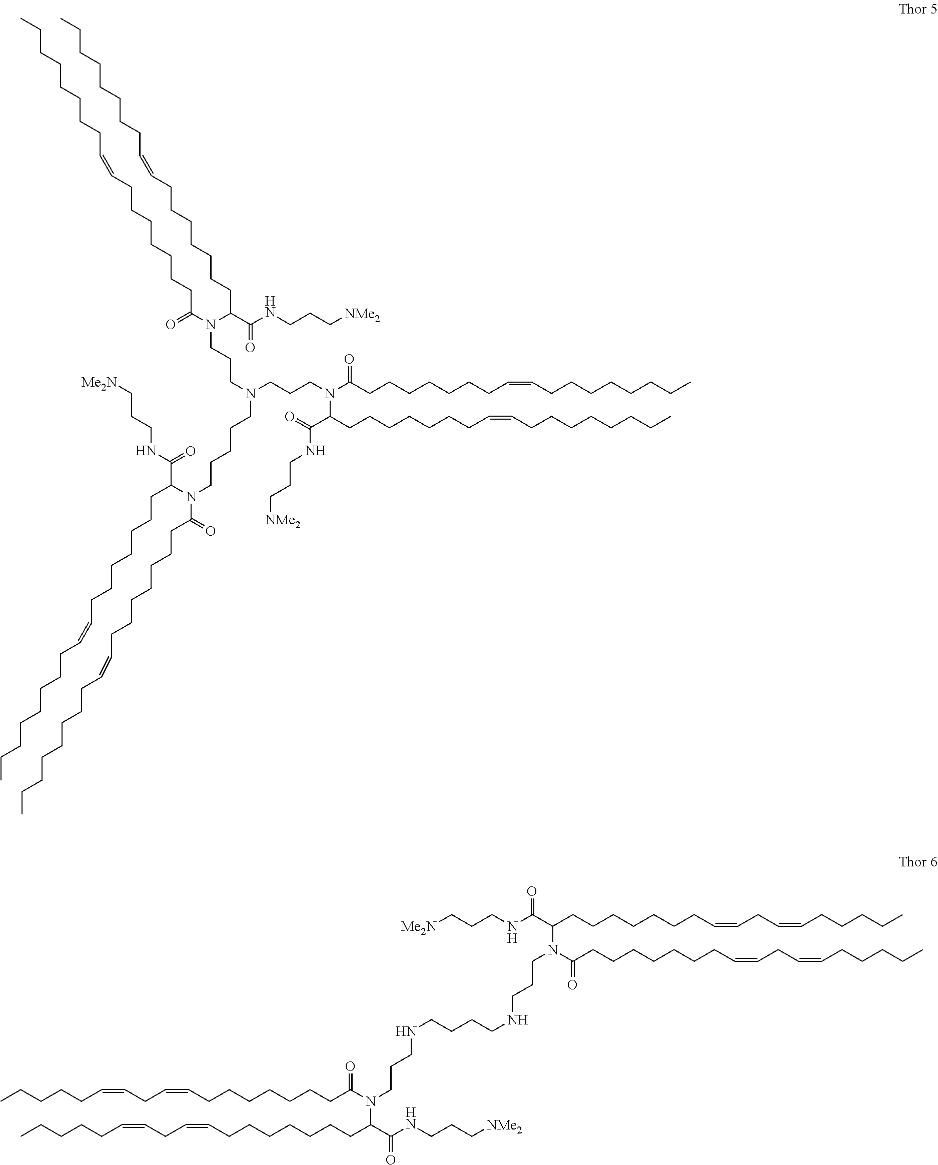

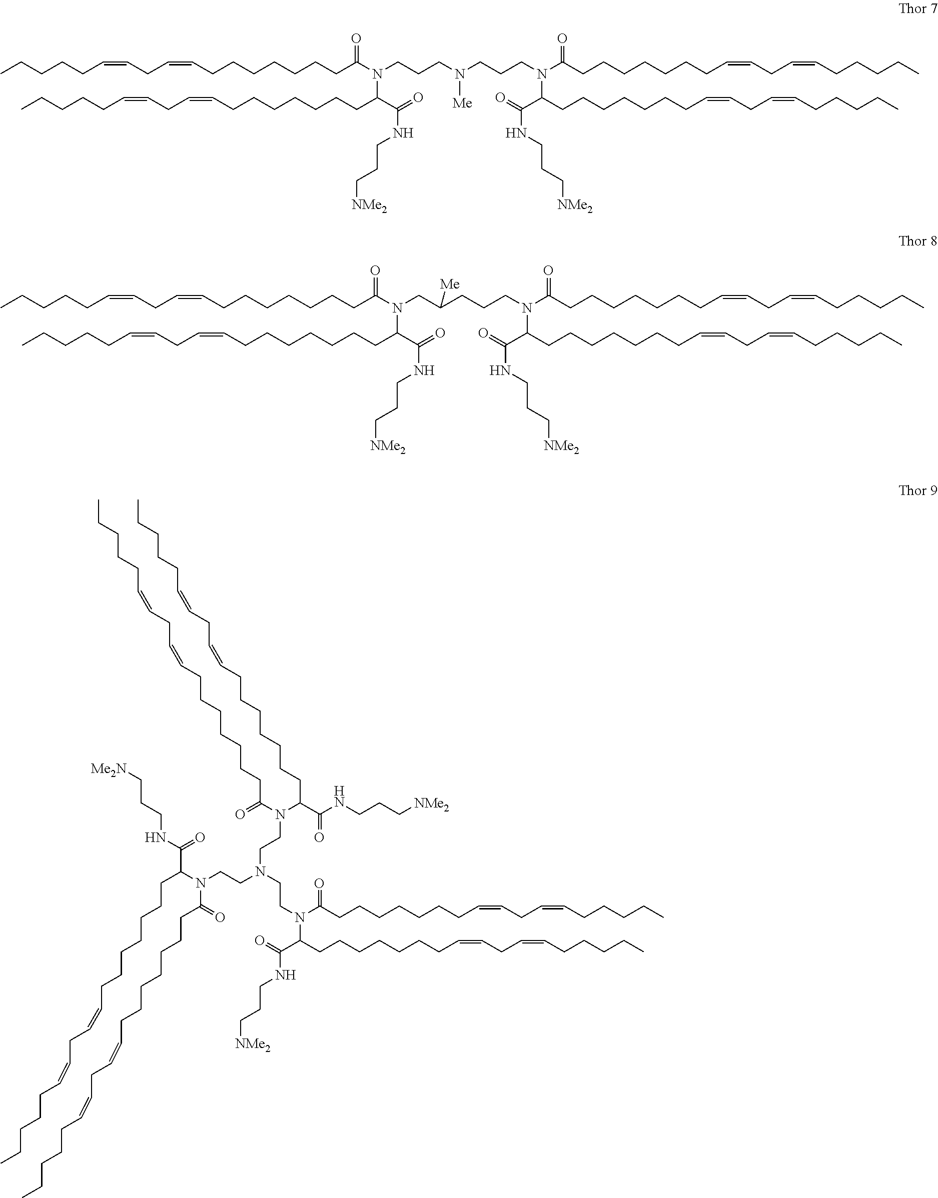

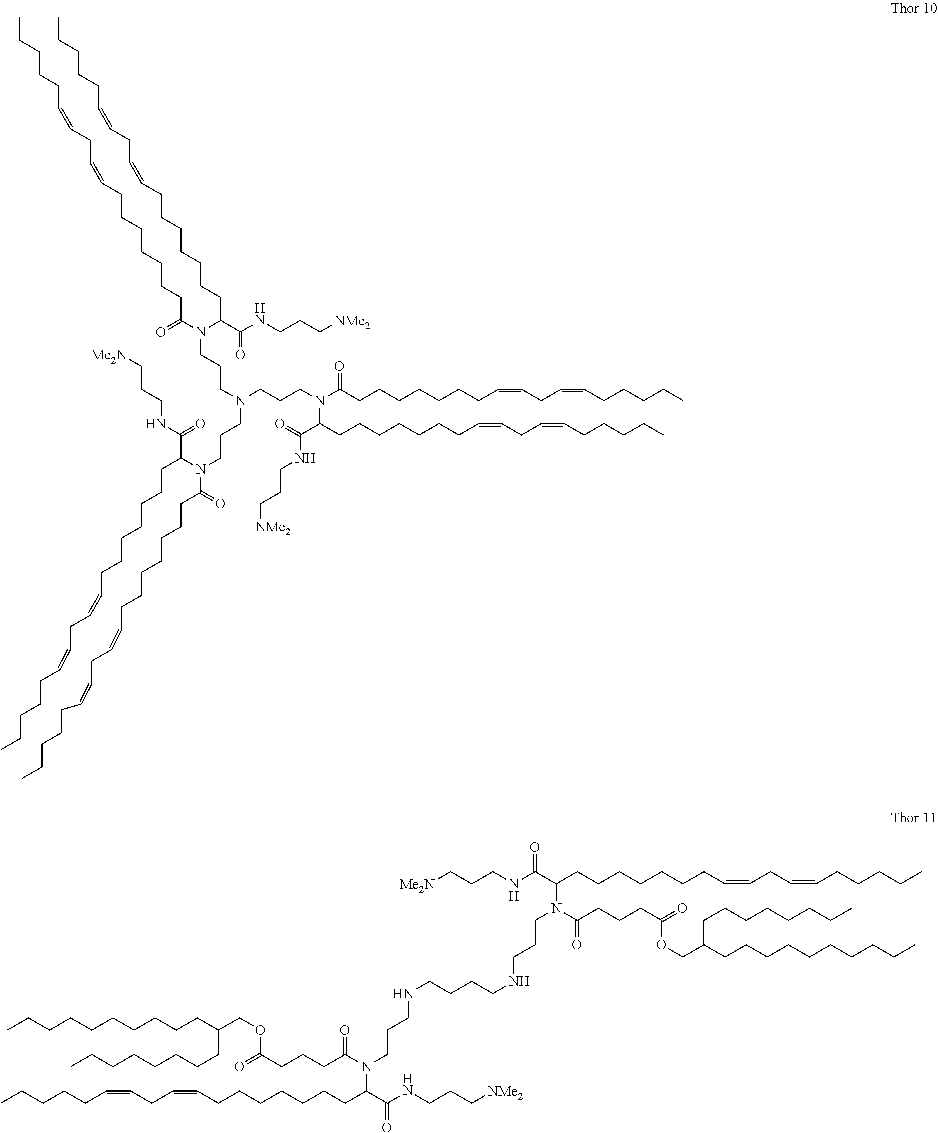

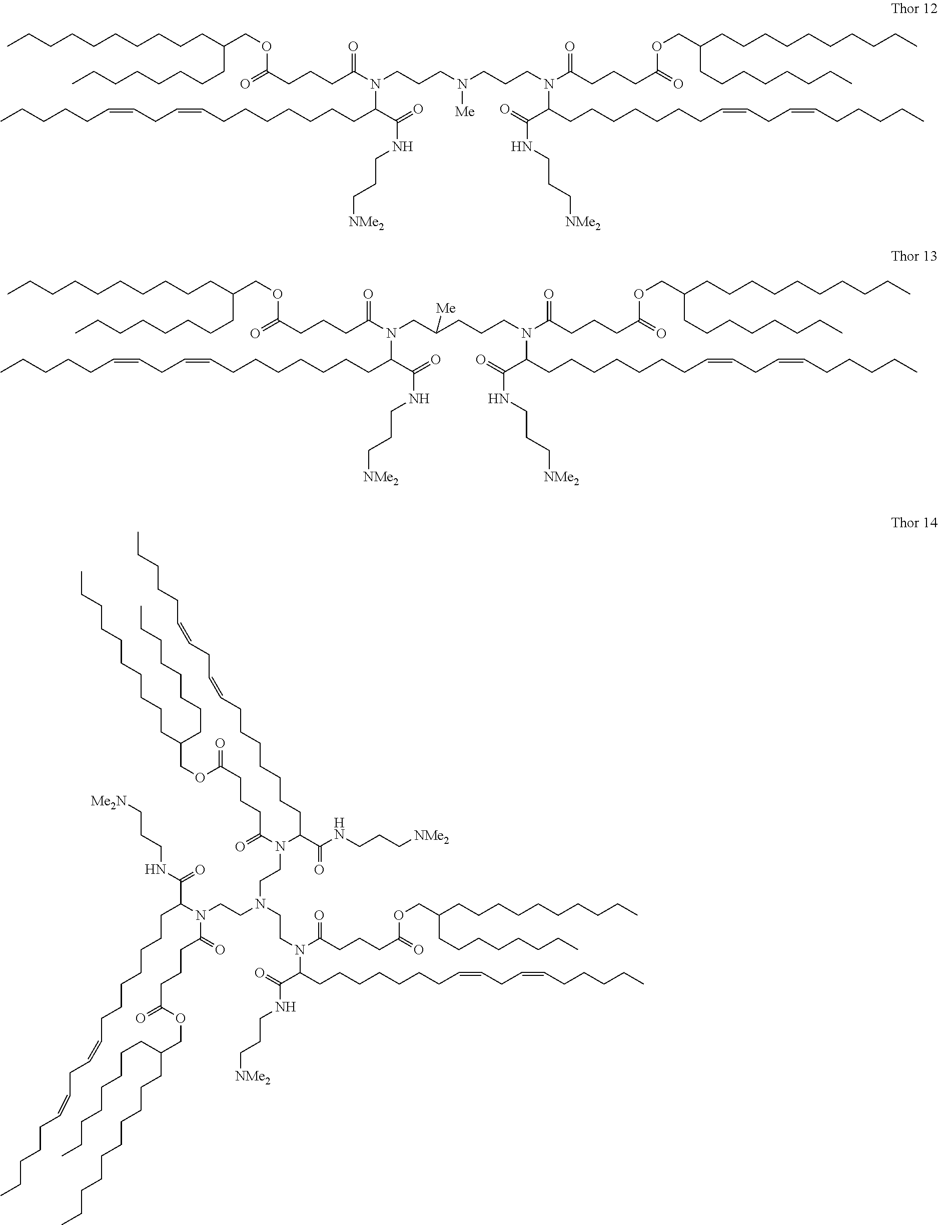

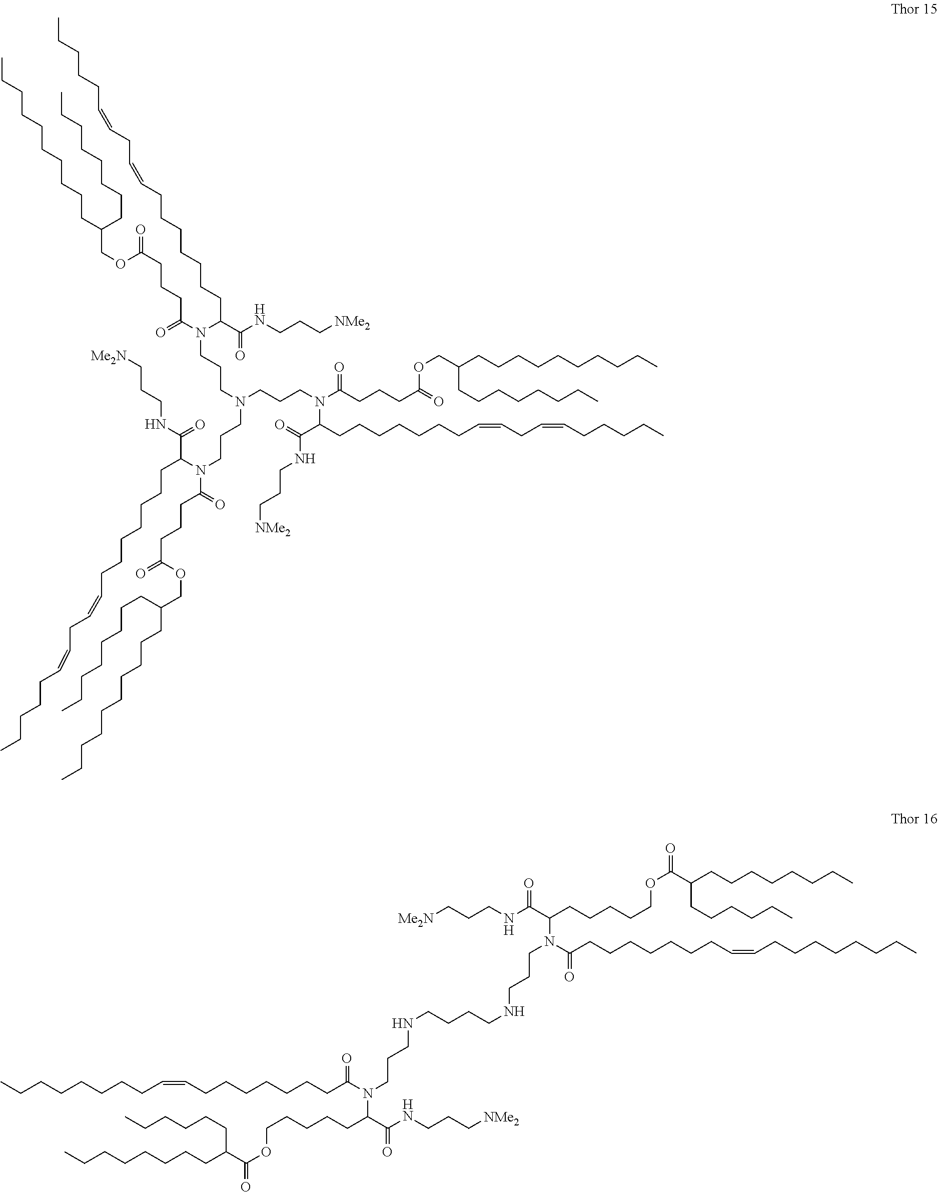

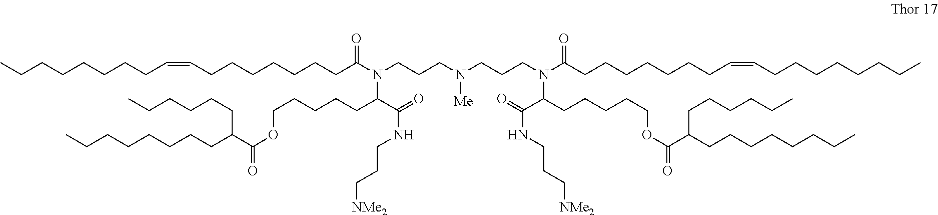 [SMILES: CCCCCCCC/C=C\CCCCCCCC(=O)N(CCCN(C)CCCN(C(=O)CCCCCCC/C=C\CCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C]

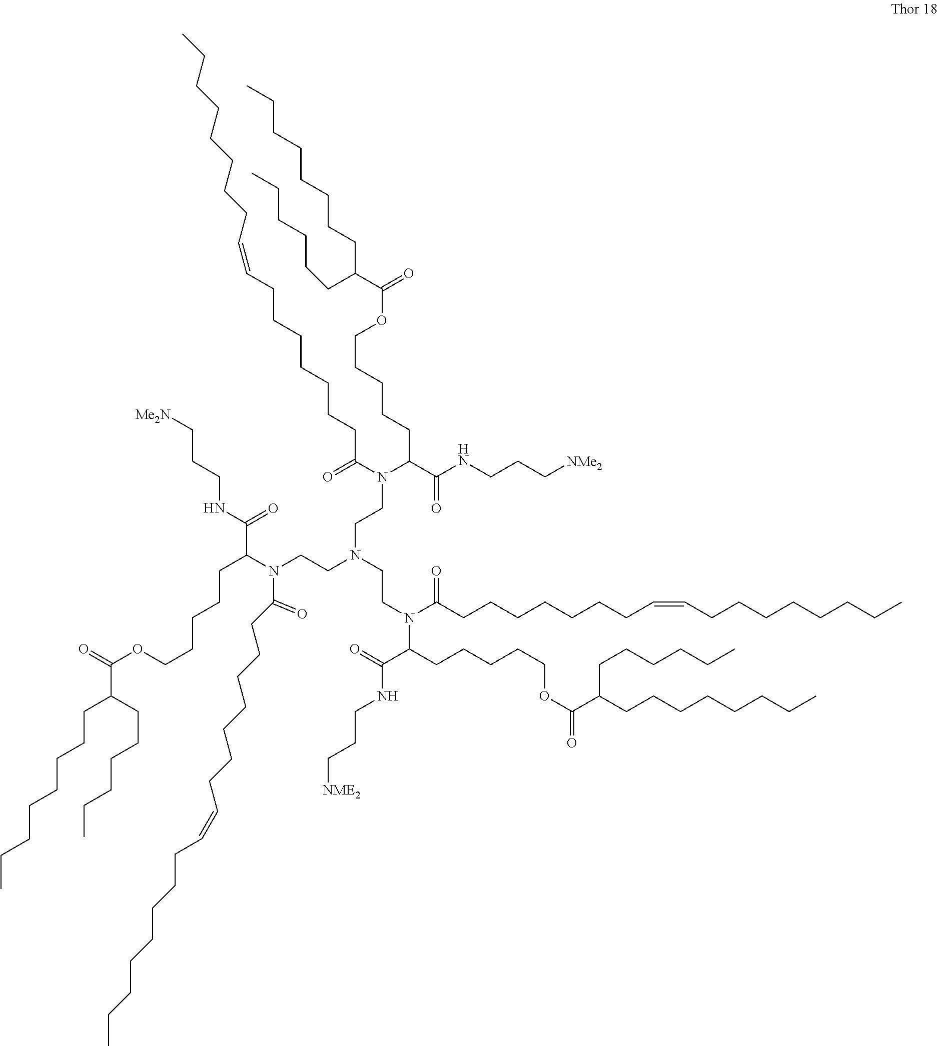

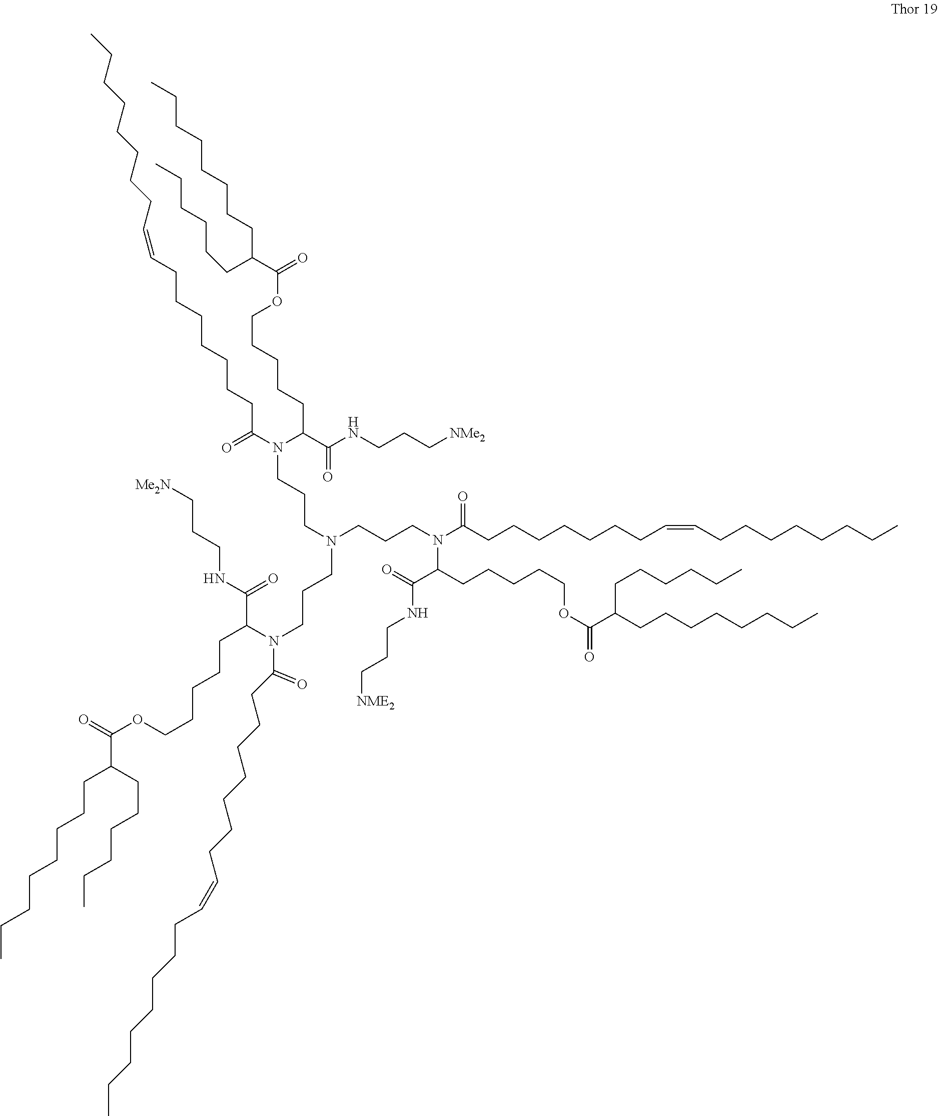

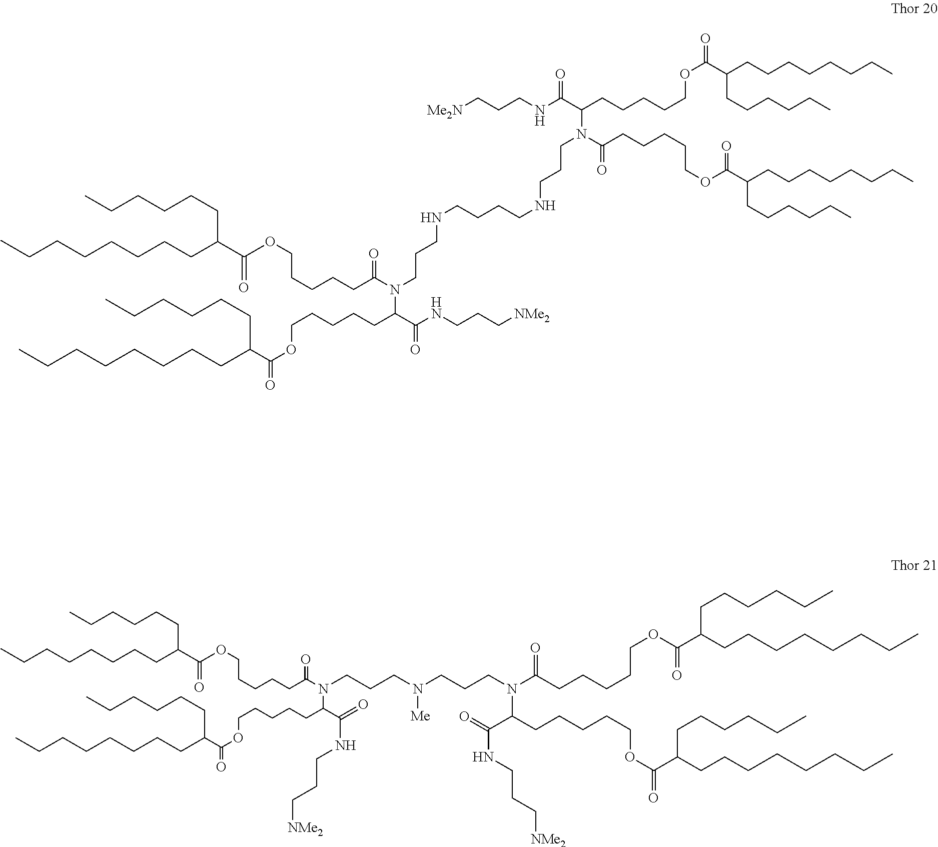

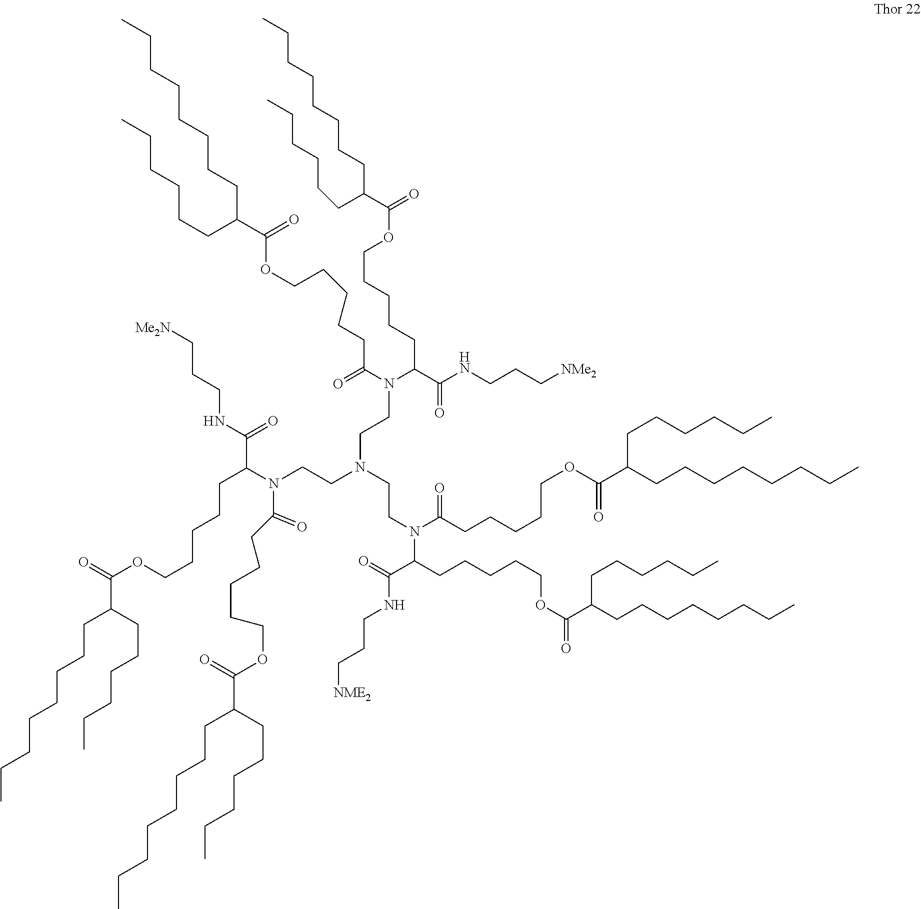

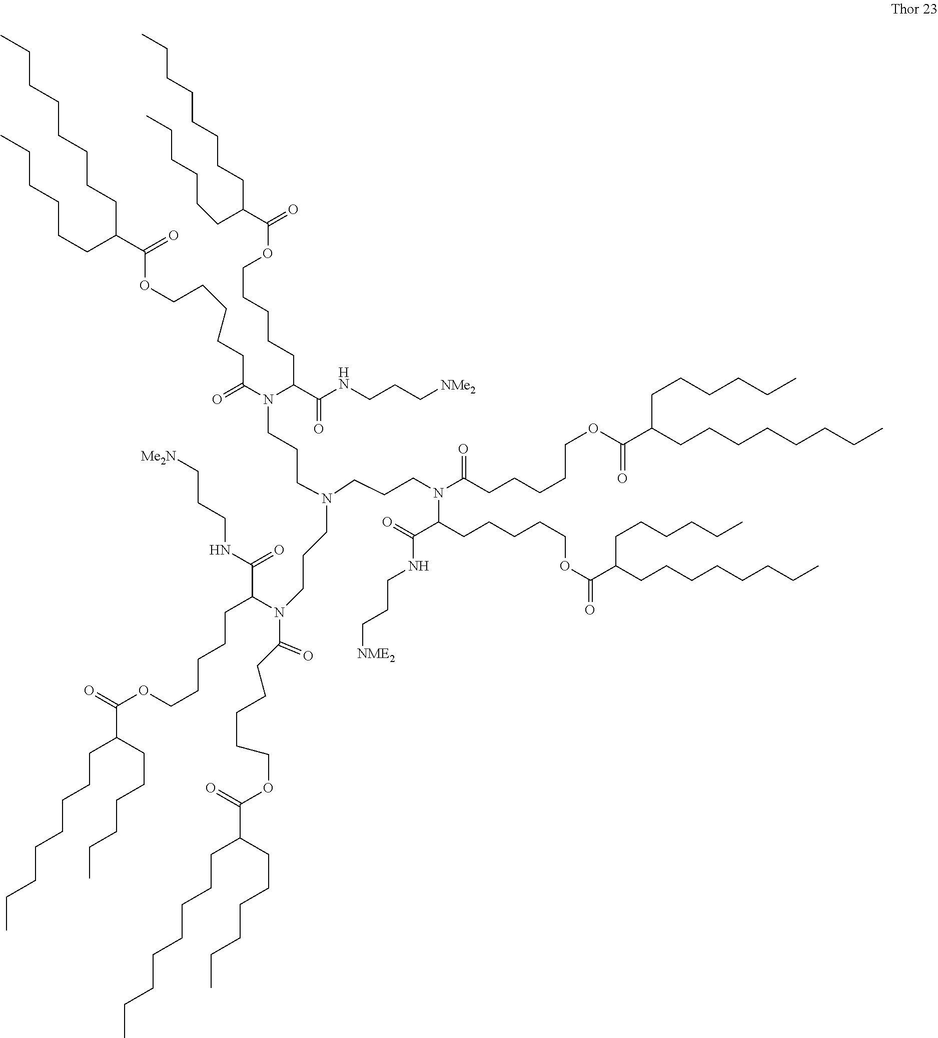

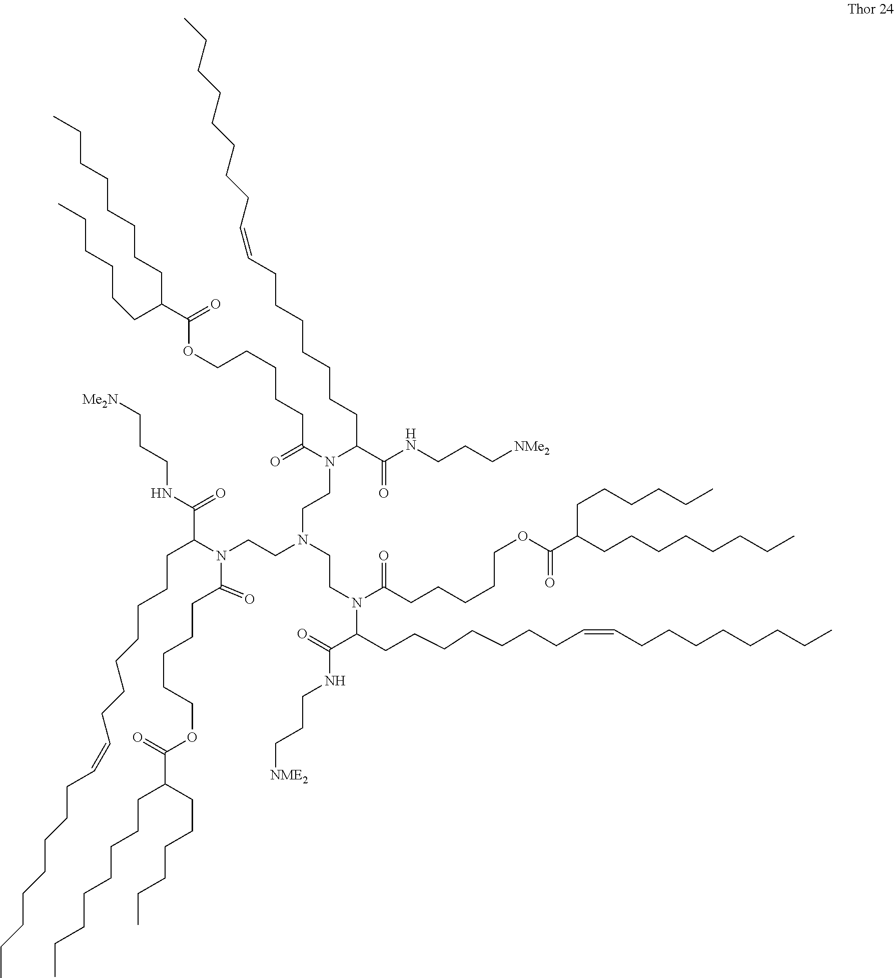

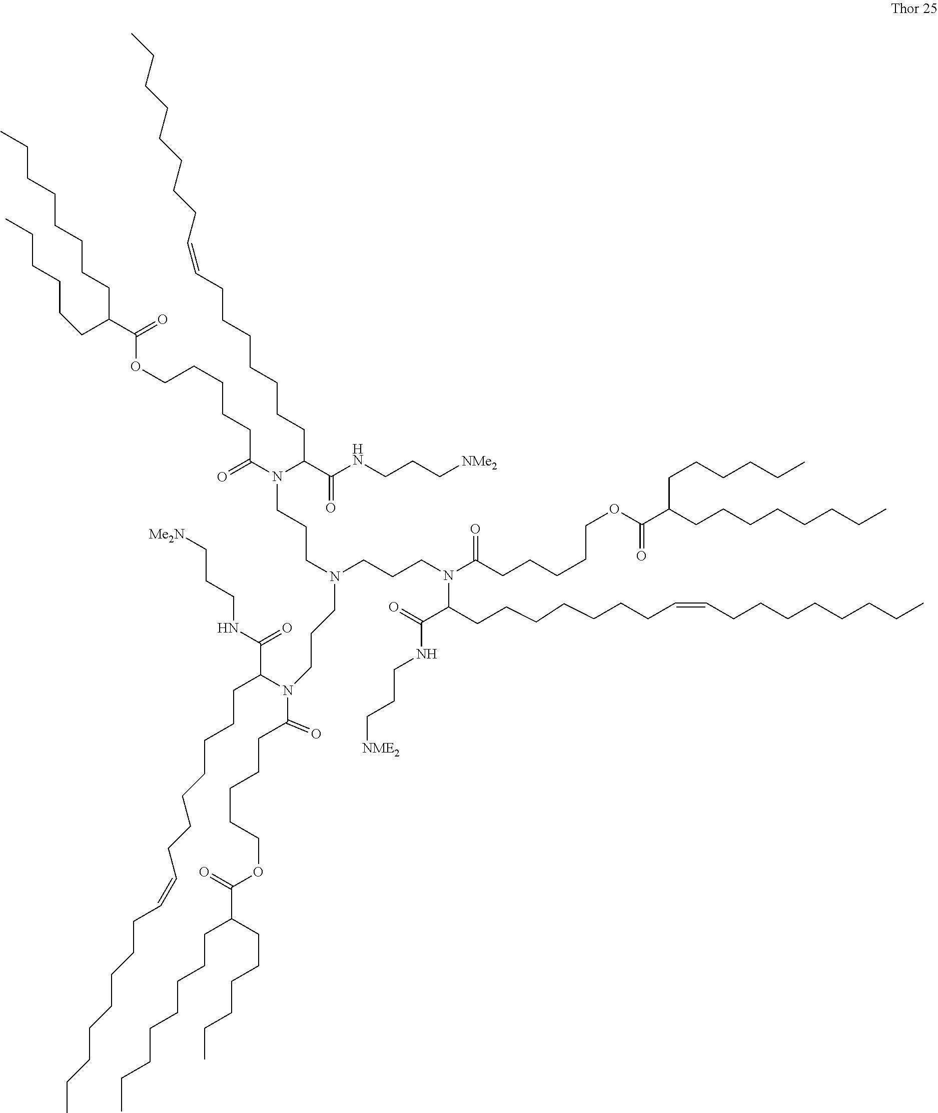

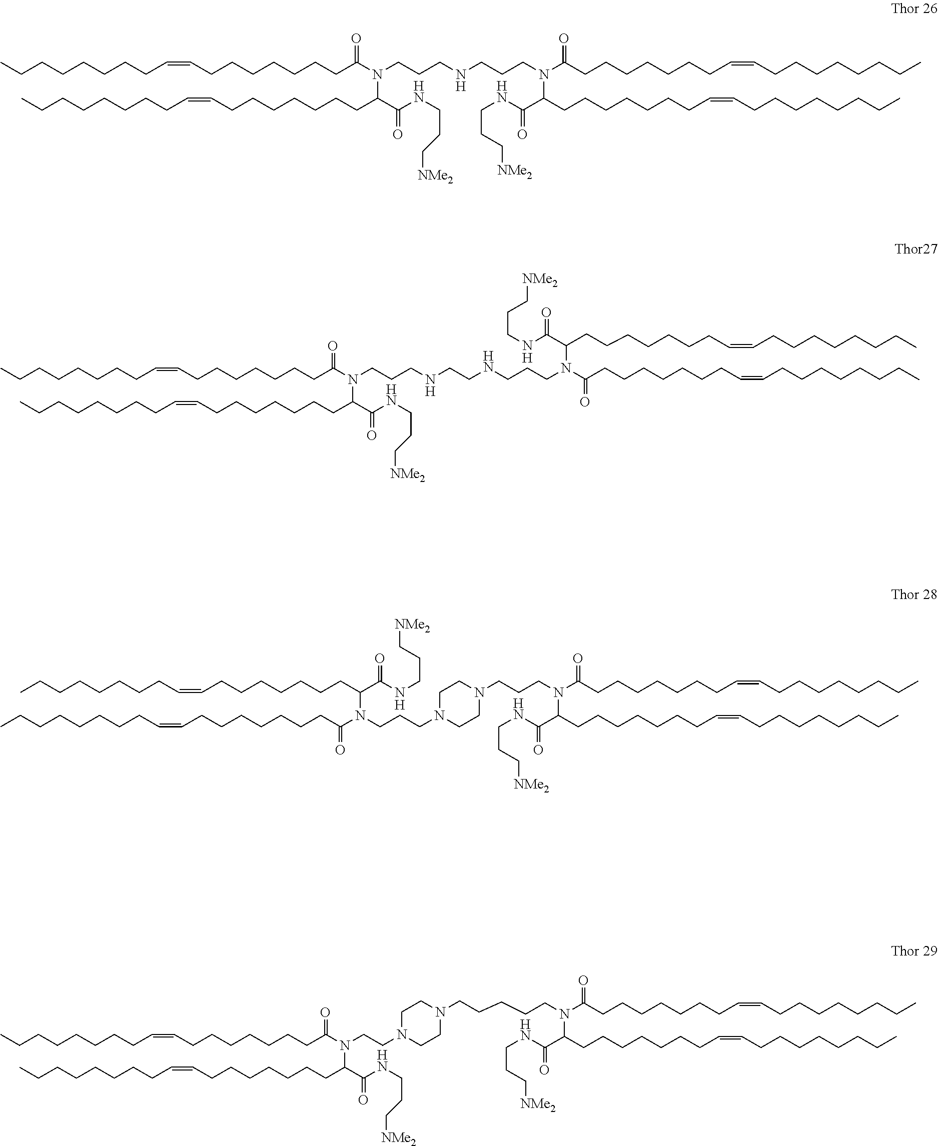

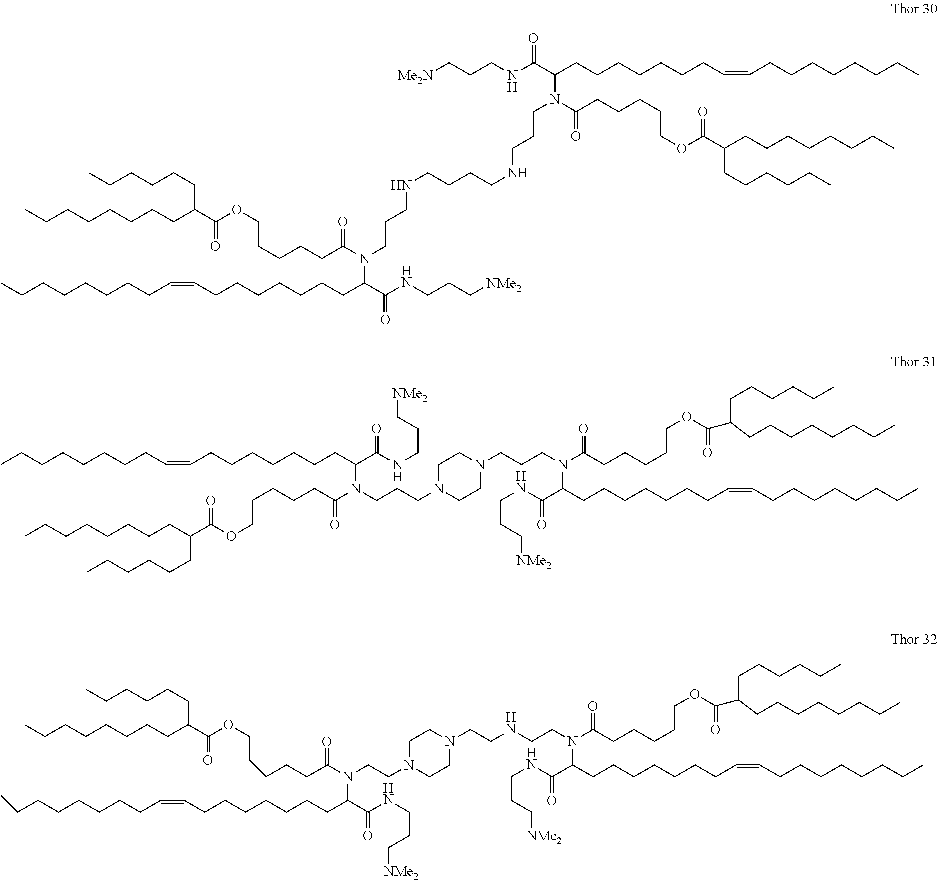

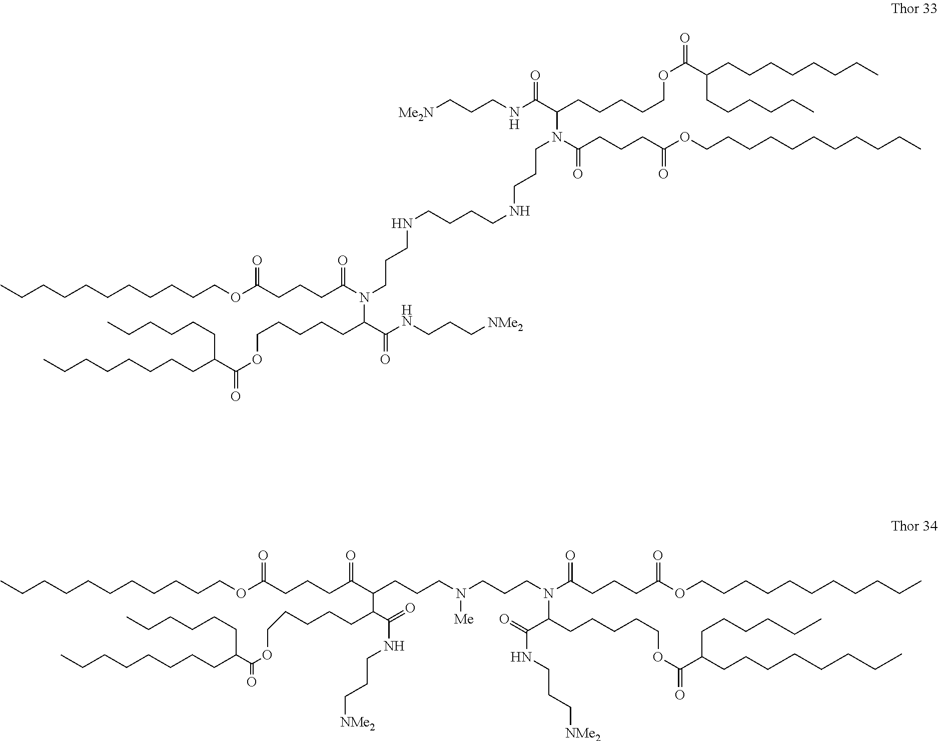

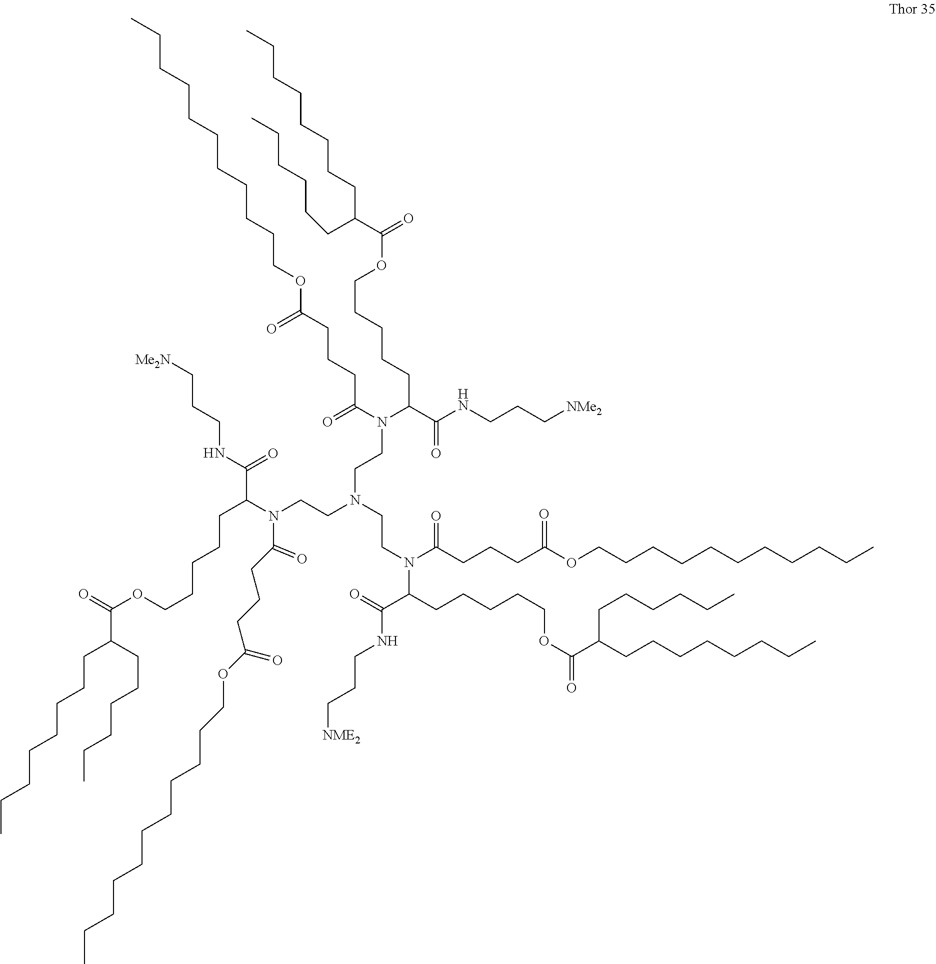

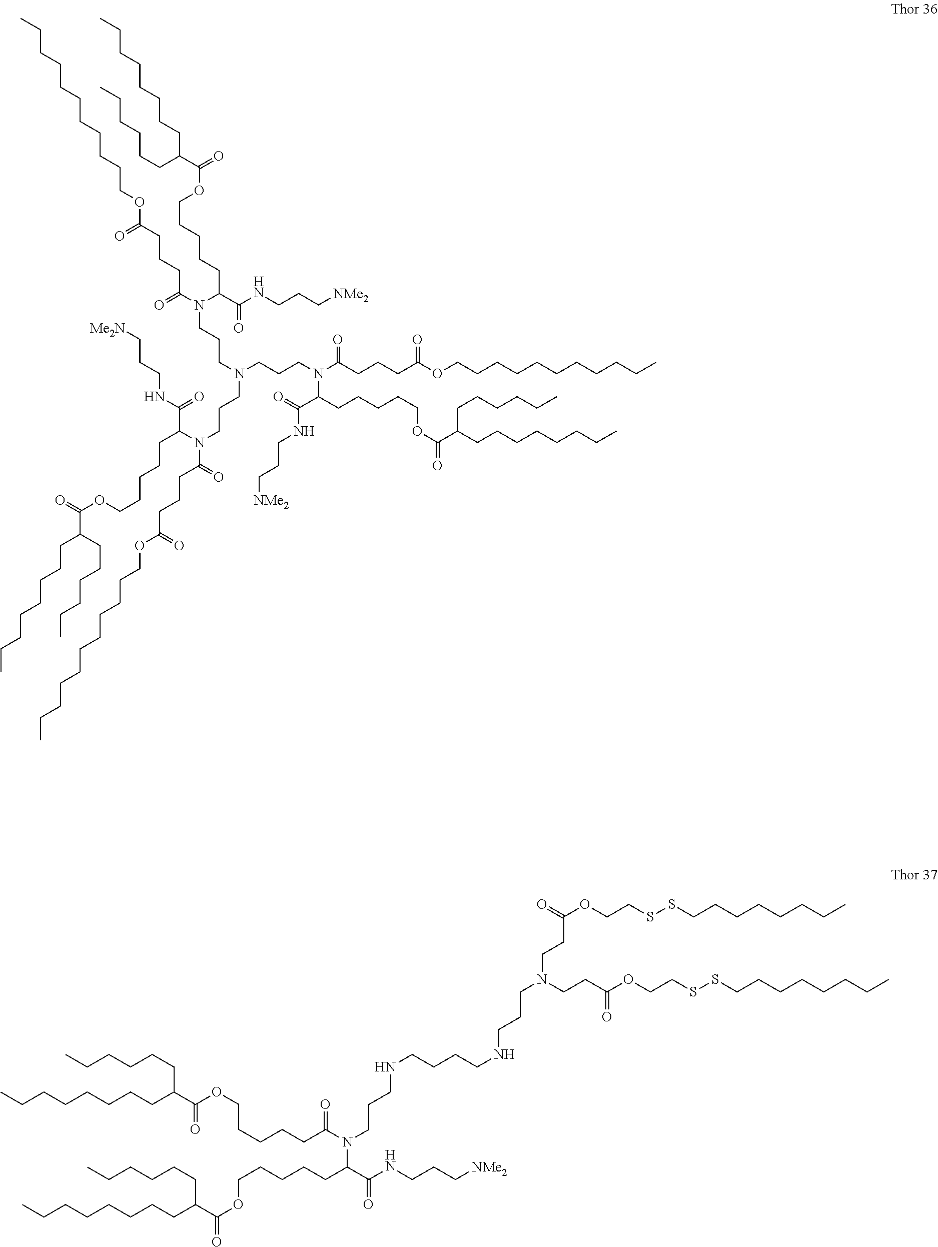

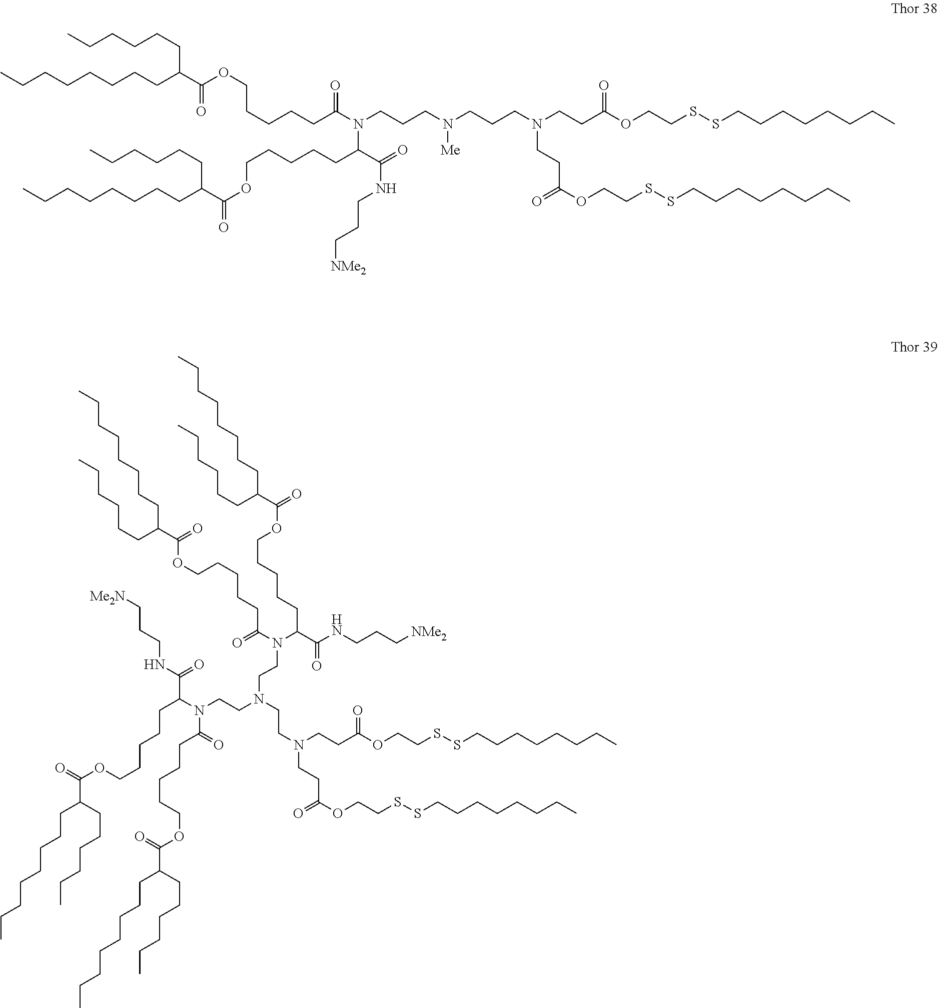 [SMILES: CCCCCCCCSSCCOC(=O)CCN(CCCN(C)CCCN(C(=O)CCCCCOC(=O)C(CCCCCC)CCCCCCCC)C(CCCCCOC(=O)C(CCCCCC)CCCCCCCC)C(=O)NCCCN(C)C)CCC(=O)OCCSSCCCCCCCC ; CCCCCCCCSSCCOC(=O)CCN(CCC(=O)OCCSSCCCCCCCC)CCN(CCN(C(=O)CCCCCOC(=O)C(CCCCCC)CCCCCCCC)C(CCCCCOC(=O)C(CCCCCC)CCCCCCCC)C(=O)NCCCN(C)C)CCN(C(=O)CCCCCOC(=O)C(CCCCCC)CCCCCCCC)C(CCCCCOC(=O)C(CCCCCC)CCCCCCCC)C(=O)NCCCN(C)C]

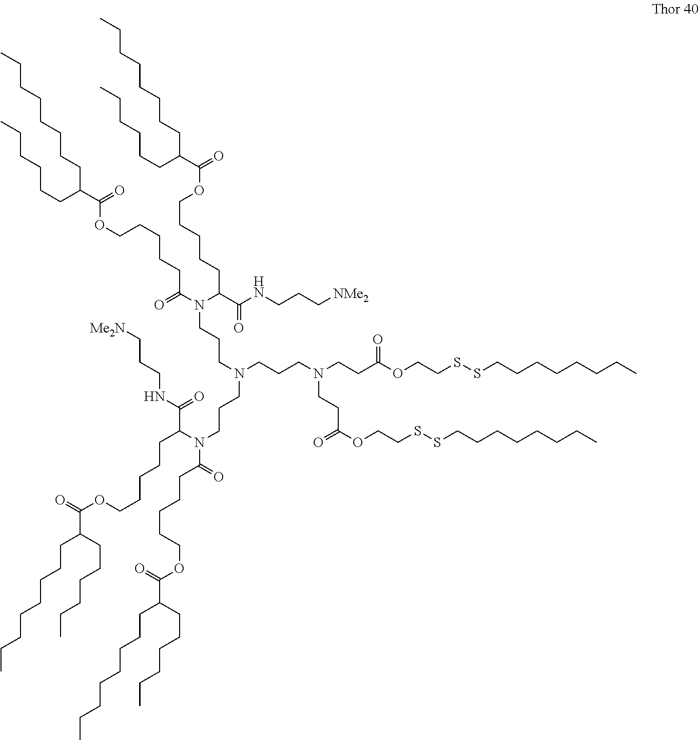 [SMILES: CCCCCCCCSSCCOC(=O)CCN(CCCN(CCCN(C(=O)CCCCCOC(=O)C(CCCCCC)CCCCCCCC)C(CCCCCOC(=O)C(CCCCCC)CCCCCCCC)C(=O)NCCCN(C)C)CCCN(C(=O)CCCCCOC(=O)C(CCCCCC)CCCCCCCC)C(CCCCCOC(=O)C(CCCCCC)CCCCCCCC)C(=O)NCCCN(C)C)CCC(=O)OCCSSCCCCCCCC]

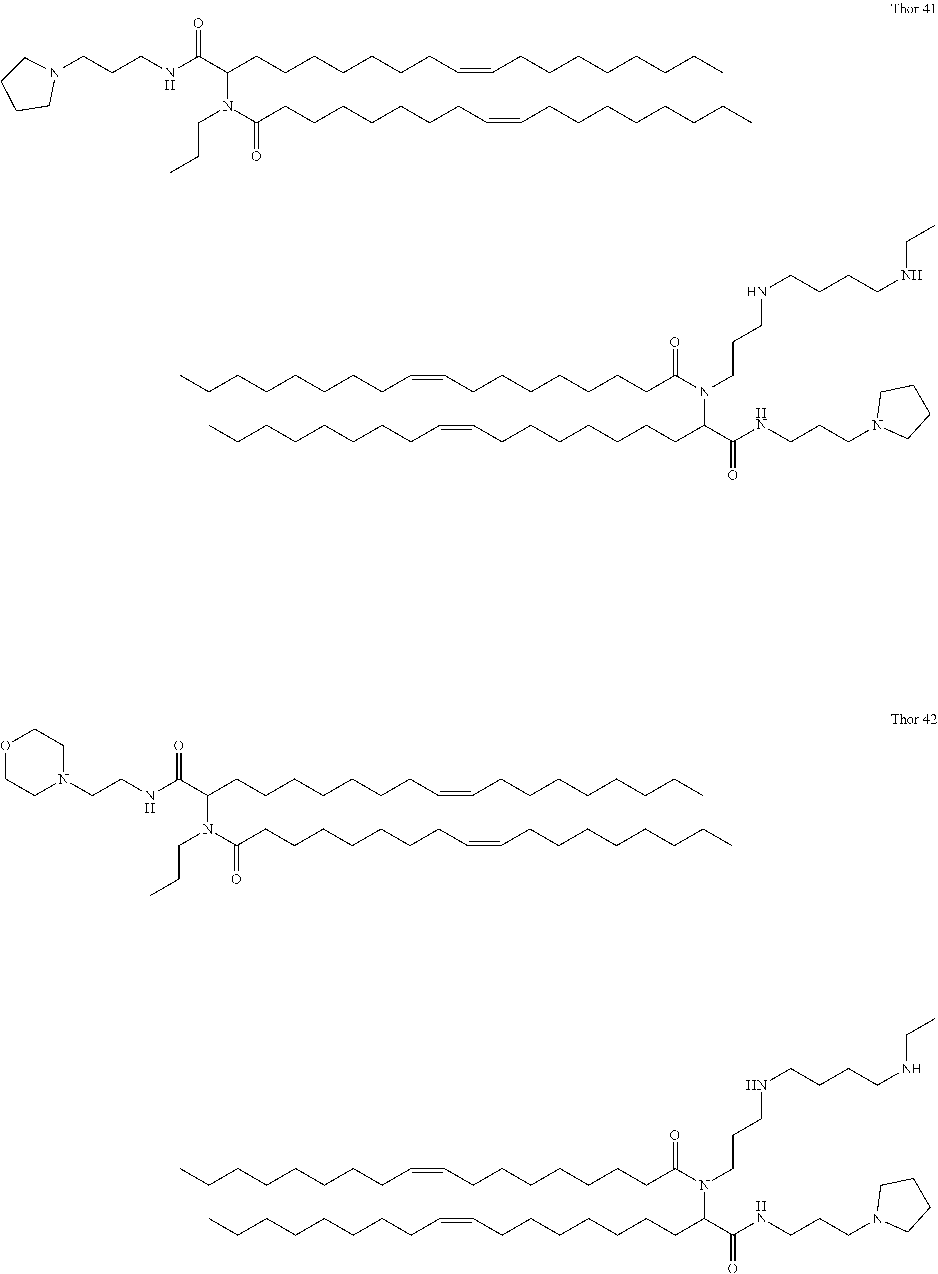

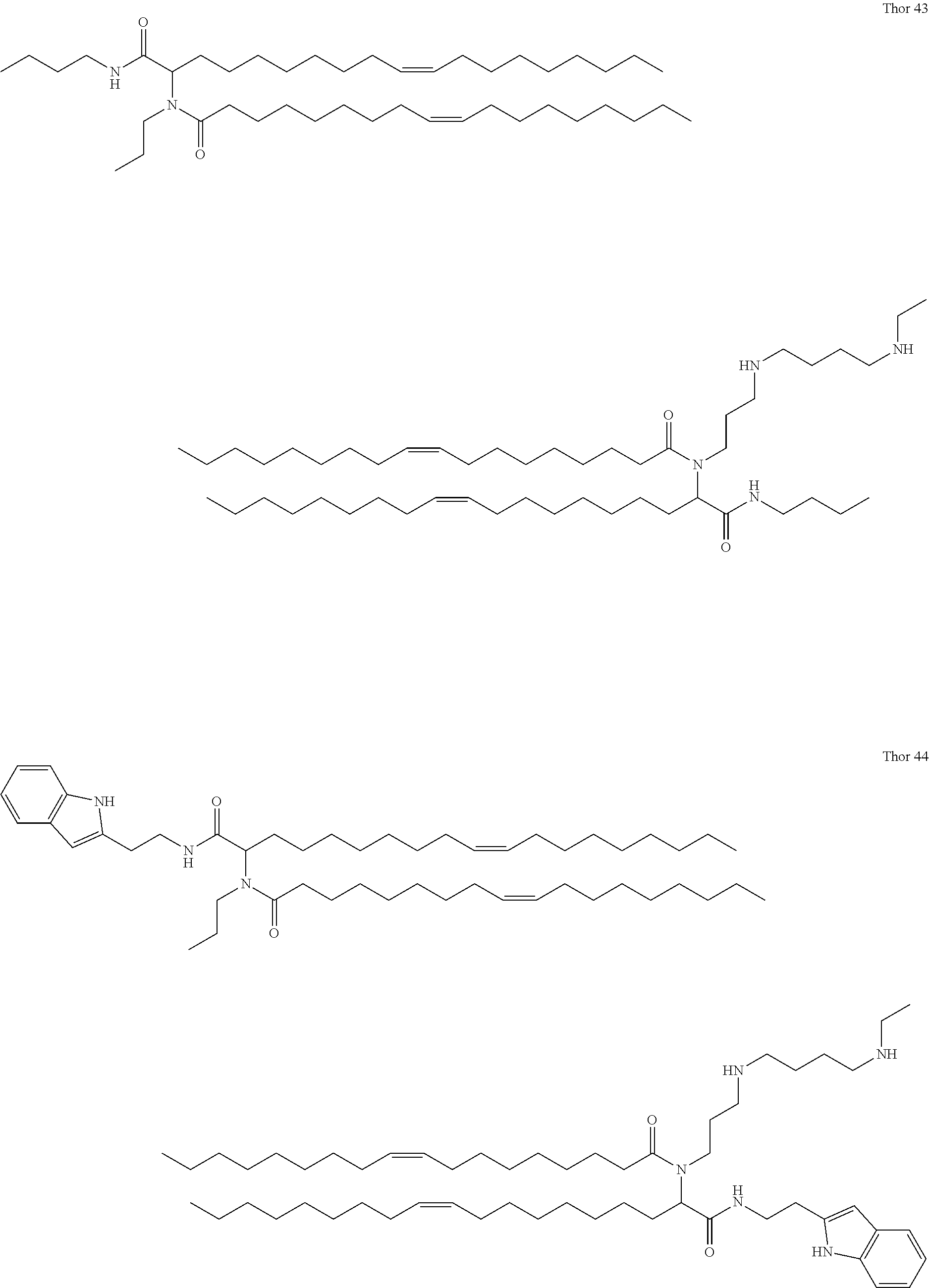

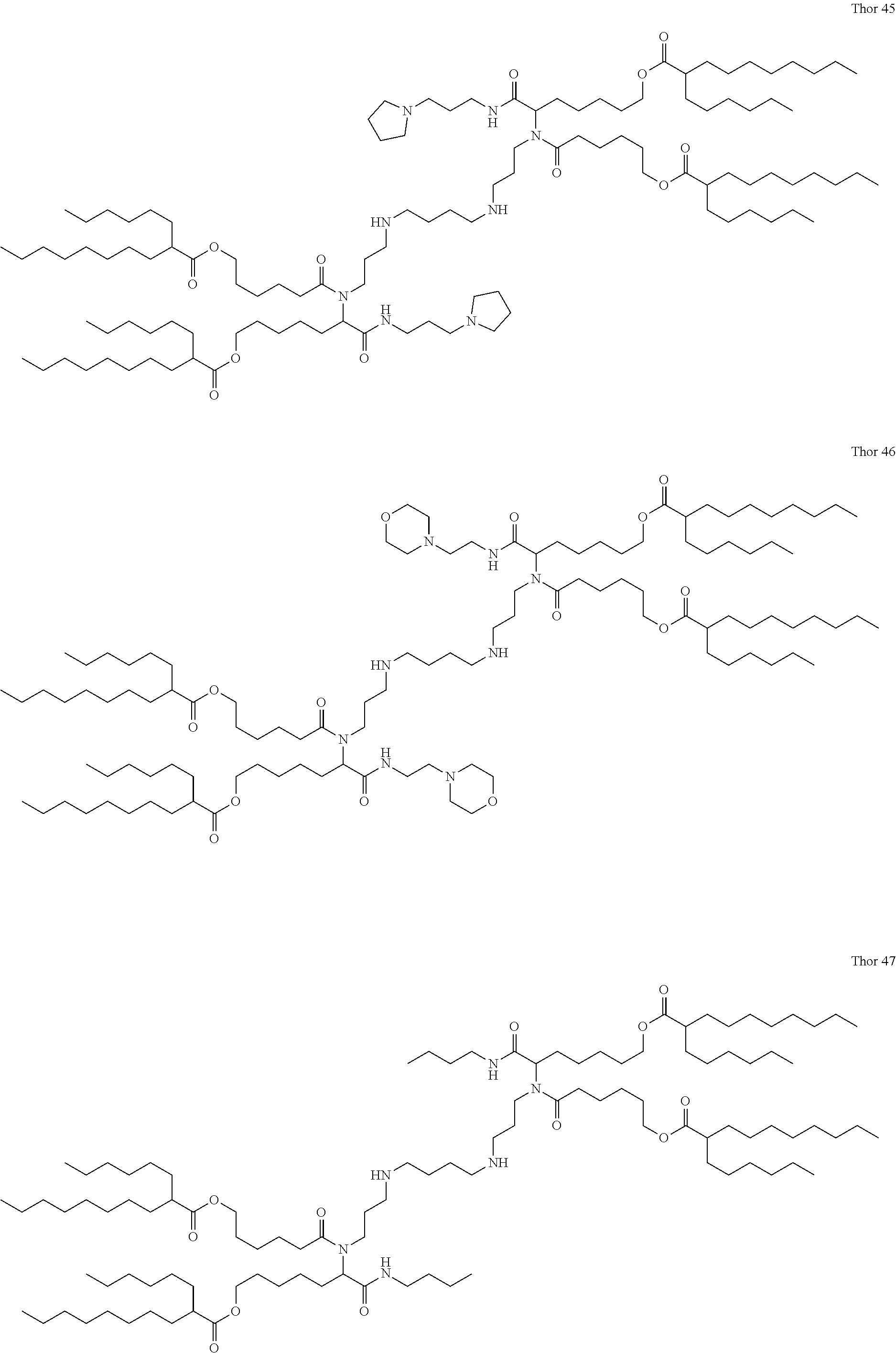

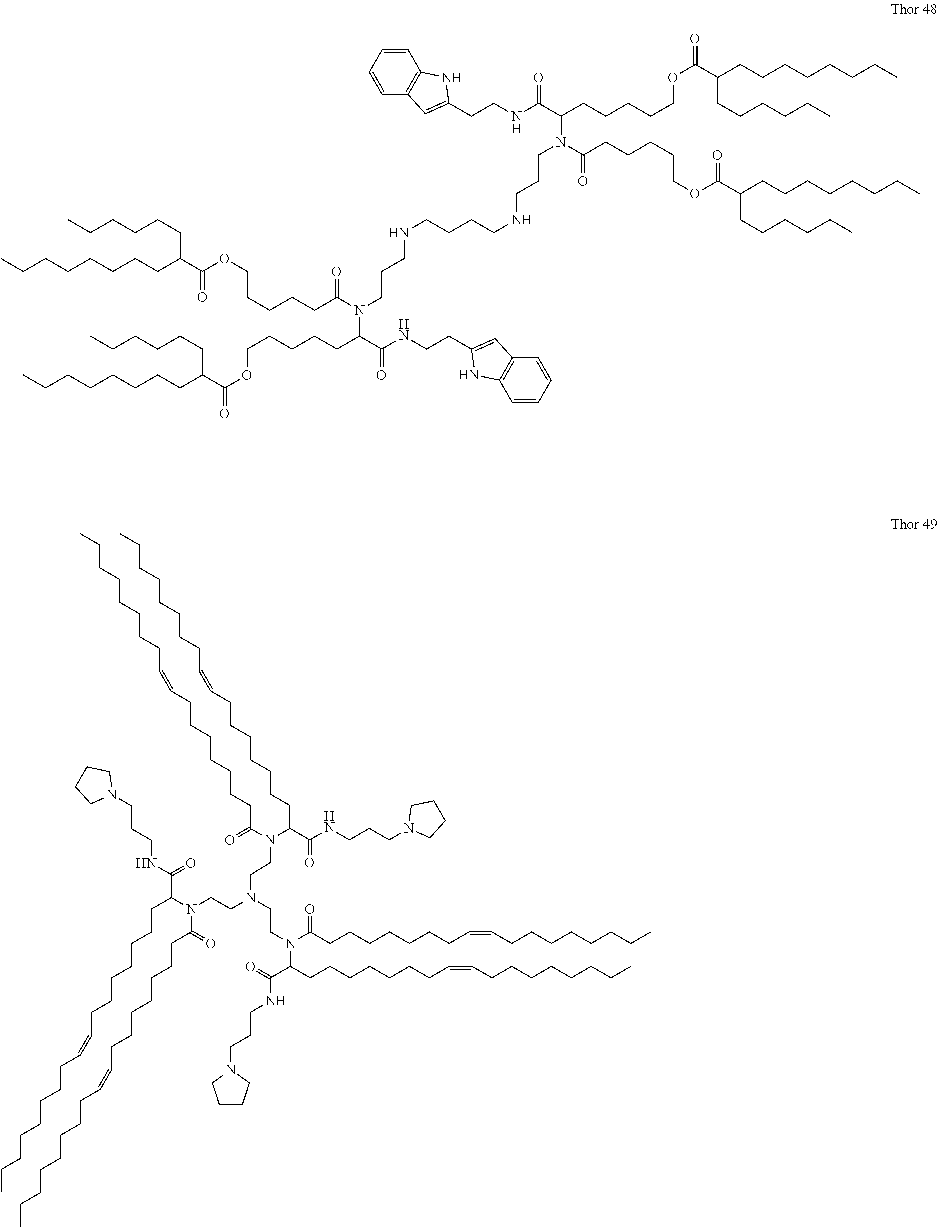 [SMILES: CCCCCCCCC(CCCCCC)C(=O)OCCCCCC(=O)N(CCCNCCCCNCCCN(C(=O)CCCCCOC(=O)C(CCCCCC)CCCCCCCC)C(CCCCCOC(=O)C(CCCCCC)CCCCCCCC)C(=O)NCCc1cc2ccccc2[nH]1)C(CCCCCOC(=O)C(CCCCCC)CCCCCCCC)C(=O)NCCc1cc2ccccc2[nH]1 ; CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCCN1CCCC1)CCN(C(=O)CCCCCCC/C=C\CCCCCCCC)C(CCCCCCC/C=C\CCCCCCCC)C(=O)NCCCN1CCCC1)C(CCCCCCC/C=C\CCCCCCCC)C(=O)NCCCN1CCCC1]

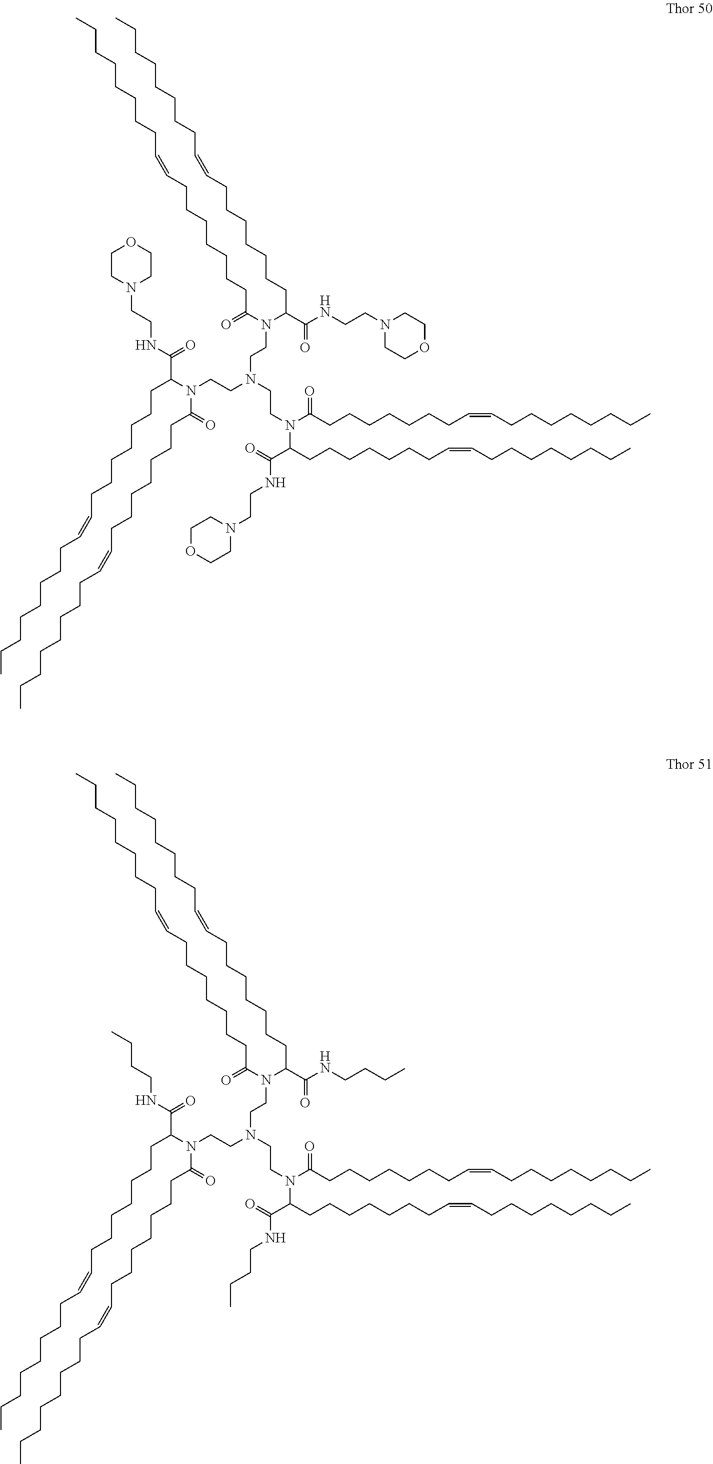 [SMILES: CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCN1CCOCC1)CCN(C(=O)CCCCCCC/C=C\CCCCCCCC)C(CCCCCCC/C=C\CCCCCCCC)C(=O)NCCN1CCOCC1)C(CCCCCCC/C=C\CCCCCCCC)C(=O)NCCN1CCOCC1 ; CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCCC)CCN(C(=O)CCCCCCC/C=C\CCCCCCCC)C(CCCCCCC/C=C\CCCCCCCC)C(=O)NCCCC)C(CCCCCCC/C=C\CCCCCCCC)C(=O)NCCCC]

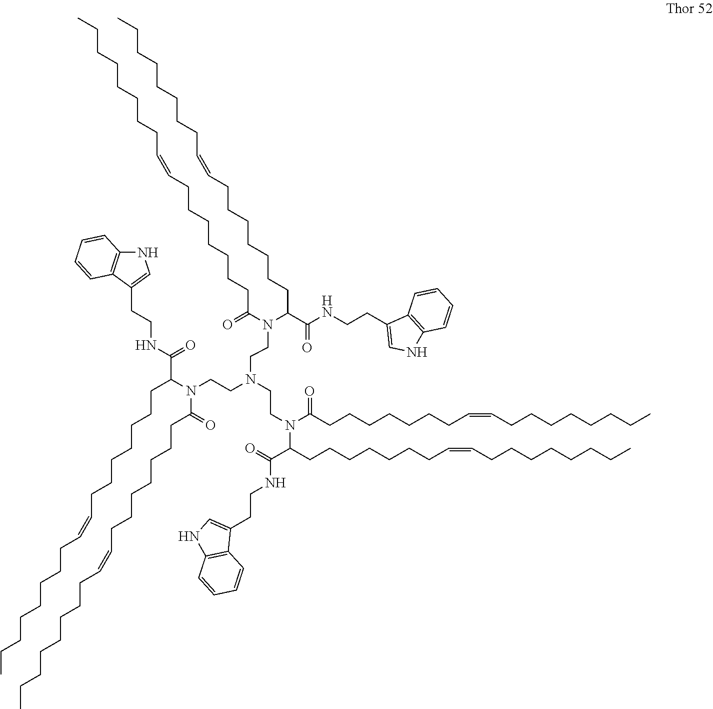 [SMILES: CCCCCCCC/C=C\CCCCCCCC(=O)N(CCN(CCN(C(=O)CCCCCCC/C=C\CCCCCCCC)C(CCCCCCC/C=C\CCCCCCCC)C(=O)NCCc1c[nH]c2ccccc12)CCN(C(=O)CCCCCCC/C=C\CCCCCCCC)C(CCCCCCC/C=C\CCCCCCCC)C(=O)NCCc1c[nH]c2ccccc12)C(CCCCCCC/C=C\CCCCCCCC)C(=O)NCCc1c[nH]c2ccccc12]

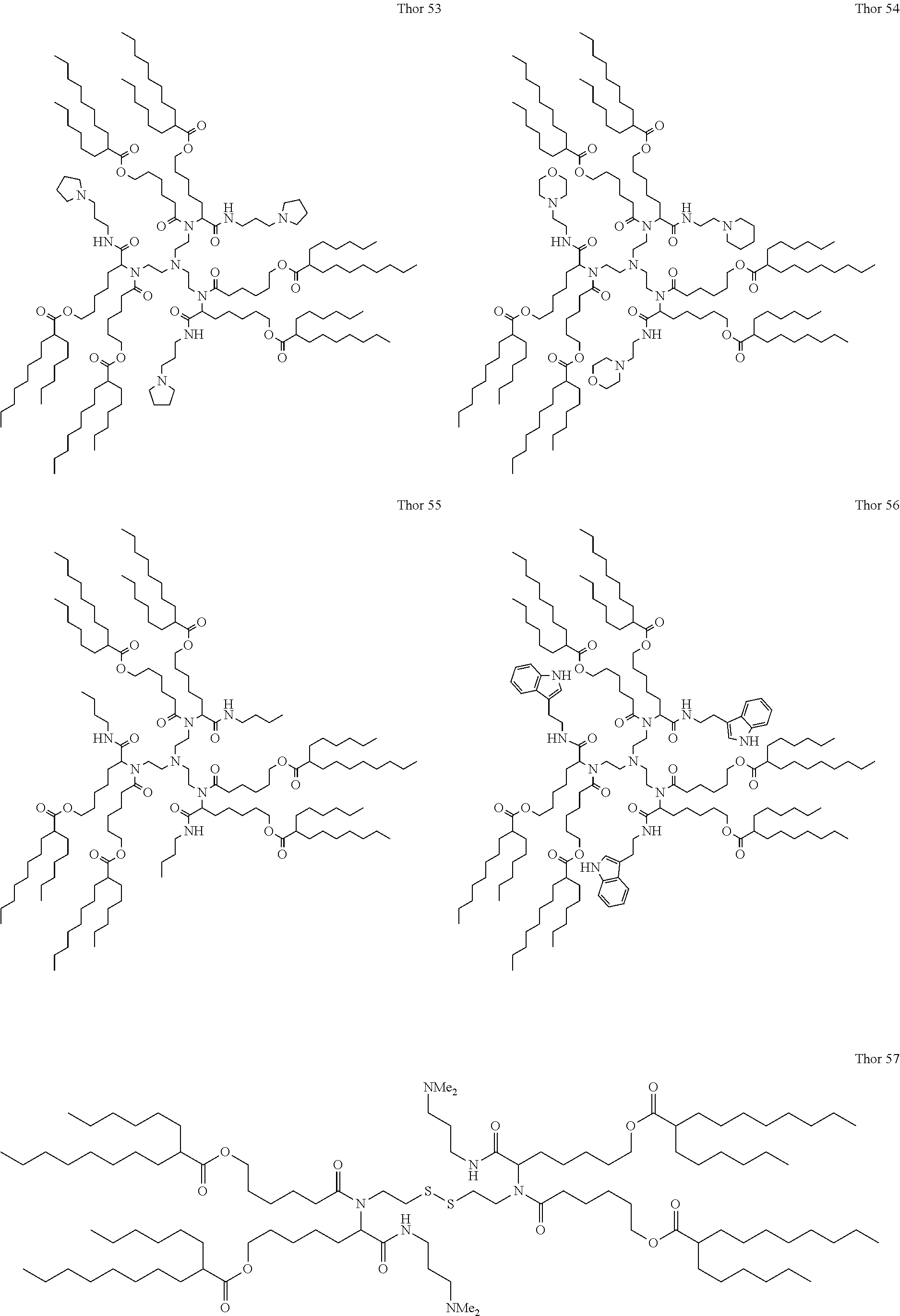

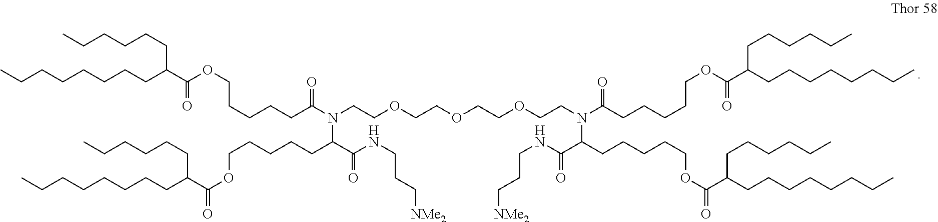 [SMILES: CCCCCCCCC(CCCCCC)C(=O)OCCCCCC(=O)N(CCOCCOCCOCCN(C(=O)CCCCCOC(=O)C(CCCCCC)CCCCCCCC)C(CCCCCOC(=O)C(CCCCCC)CCCCCCCC)C(=O)NCCCN(C)C)C(CCCCCOC(=O)C(CCCCCC)CCCCCCCC)C(=O)NCCCN(C)C]

17. A composition comprising a compound according to claim 1, a phospholipid, a structural lipid, a polymer-conjugated lipid, and an agent selected from a nucleic acid, small molecule drug, protein, polypeptide, antibody, peptide, or a combination thereof.

18. The composition of claim 17, wherein the composition comprises:
the phospholipid in an amount of from greater than zero to 30 mol % of the total amount of lipids;
the structural lipid in an amount of from greater than zero to 70 mol % of the total amount of lipids;
the polymer-conjugated lipid in an amount of from greater than zero to 10 mol % of the total amount of lipids; or
any combination thereof.

19. The composition of claim 17, wherein:
the phospholipid is selected from 1,2-dioleoyl-sn-glycero-3-phosphoethanolamine (DOPE), 1,2-distearoyl-sn-glycero-3-phosphocholine (DSPC)1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), or a combination thereof;
the structural lipid is selected from cholesterol, beta-sitosterol, cholestanol, fucosterol, campesterol, stigmastanol, brassicasterol, ergosterol, stigmasterol, or a combination thereof; and
the polymer-conjugated lipid is selected from a PEG-modified phosphatidylethanolamine, PEG-modified phosphatidic acid, PEG-modified phosphoethanolamine, PEG-modified ceramide (PEG-CER), PEG-modified dialkylamine, PEG-modified diacylglycerol (PEG-DAG), PEG-modified dialkylglycerol, or a combination thereof.

20. A method of using a composition according to claim 17, comprising administering an effective amount of the composition to a subject.
